(19) United States
(12) Patent Application Publication
(10) Pub. No.: US 2023/0159887 A1
(43) Pub. Date: May 25, 2023

(54) Methods for Generating Thymic Cells in Vitro

(71) Applicant: The Regents of the University of California, Oakland, CA (US)

(72) Inventors: Audrey Parent, San Francisco, CA (US); Matthias Hebrok, Belmont, CA (US); Mark Stuart Anderson, Larkspur, CA (US)

(21) Appl. No.: 17/921,739

(22) PCT Filed: Apr. 27, 2021
(86) PCT No.: PCT/US2021/029457

§ 371 Date: Oct. 27, 2022

Related U.S. Application Data
(60) Provisional application No. 63/147,126, filed on Feb. 8, 2021.
(60) Provisional application No. 63/016,527, filed on Apr. 28, 2020.

(51) Int. Cl. C12N 5/071; A61K 35/26

(52) U.S. Cl. CPC C12N 5/0617; A61K 35/26; C12N 2506/45; C12N 2501/16; C12N 2501/415; C12N 2501/155; C12N 2501/119; C12N 2501/15; C12N 2501/30; C12N 2500/25

(57) ABSTRACT

The present description provides improved methods for generating thymic epithelial progenitor (TEP) cells from pluripotent stem (PS) cells in vitro. Also provided are isolated invitro cell populations, compositions, and systems comprising TEP cells produced in vitro. Compositions and systems of cell populations of thymic epithelial cells and subpopulations thereof, as well as cells formed during different stages of differentiation of PS cells into thymic epithelial cells and subpopulations thereof are provided.

### CROSS-REFERENCE TO RELATED APPLICATIONS

This application claims priority to U.S. provisional patent application No. 63/147,126 filed on Feb. 8, 2021 entitled Methods for Generating Thymic Cells In Vitro and U.S. provisional patent application No. 63/016,527 filed on Apr. 28, 2020 entitled Methods for Generating Thymic Cells In Vitro, the contents of each of which are herein incorporated by reference in their entirety.

### GOVERNMENT LICENSE RIGHTS

This invention was made with government support under grant U01 DK107383 awarded by the National Institutes of Health. The government has certain rights in the invention.

### FIELD OF THE DISCLOSURE

The present disclosure relates to compositions and methods for generating thymic cells including, e.g., thymic epithelial cells.

### BACKGROUND

The use of stem cells to replace lost or damaged tissue remains a promising field of research for development of therapeutic compositions and methods. In particular, thymic epithelial progenitor cells derived from stem cells present an increasingly important application of stem cell-based therapeutic approaches for treatment of a wide variety of disease.

The thymus is a primary lymphoid organ that plays a central role in the immune system. For example, the microenvironment of the thymus provides a unique training ground for the development and maturation of effector cells such as lymphocytes (e.g., T cells). The thymus is also the main organ involved in establishing immune tolerance through the elimination of autoreactive T cell subsets and through the production of regulatory T cells (reviewed in Anderson et al., Nat Rev Immunol 7, 954-963, 2007). These critical functions are mediated by thymic epithelial cells, the main component of the thymic stroma.

There remains a need for improved methods of generating functional thymic epithelial progenitor (TEP) cells and for cell populations enriched in functional TEP cells that can differentiate into functional thymic epithelial cells. In particular, there remains a critical need for recapitulating a complex thymic microenvironment to support the maturation and function of thymic cells in vitro for use in therapeutic applications.

### SUMMARY OF THE DISCLOSURE

Methods of generating thymic epithelial progenitor (TEP) cells in vitro are provided, for example, in International Patent Application Publication Number WO2014134213, the disclosure of which is incorporated by reference herein in its entirety. Given the thymus' central role in the immune system, thymic cells and thymic tissue have great therapeutic potential.

Accordingly, provided herein are methods for generating thymic epithelial progenitor (TEP) cells in vitro comprising culturing a population of cells in a first medium comprising an activator of bone morphogenetic protein (BMP) signaling, an activator of fibroblast growth factor (FGF) signaling, and an inhibitor of transforming growth factor-β (TGF-β) signaling, and further culturing the population of cells to induce further maturation of the TEP cells in vitro, wherein the further culturing comprises culturing the population of cells in a second medium comprising keratinocyte growth factor (KGF), heparin, and hydrocortisone. The cells produced from further maturation of TEP cells may be referred to as mature TEP (mTEP) cells.

Further culturing the population of cells can be performed in a medium further comprising a triiodo-L-thyronine (T3) supplement.

Further culturing the population of cells can be performed in a medium further comprising an insulin-transferrin-selenium (ITS) supplement.

Further culturing the population of cells can be performed in a medium further comprising a B27 supplement.

The population of cells can comprise one or more of definitive endodermal (DE) cells, anterior foregut endodermal (AFE) cells, ventral pharyngeal endodermal (VPE) cells, and TEP cells.

Further culturing the population of cells to induce further maturation of the TEP cells in vitro can comprise further culturing the population of cells for up to 14 days.

Further culturing the population of cells to induce further maturation of the TEP cells in vitro can comprise transferring the population of cells to an extracellular matrix-based medium such as Matrigel.

The methods can be performed in a cell culture medium, wherein the first and/or second medium is a liquid medium and the culture conditions comprise suspension culture.

The first and/or second medium can be a minimum essential medium or Dulbecco's minimum essential medium (DMEM).

The methods can further comprise transplanting the TEP cells to a subject. In such embodiments, further culturing the population of cells to induce further maturation of the TEP cells in vitro gives rise to thymic epithelial cells (TECs) comprising subpopulations of TECs after transplantation in vivo.

The subpopulations of TECs can comprise one or more of cortical thymic epithelial cell (cTEC) lineage cells, bipotent TEP cells, committed medullary thymic epithelial cell (mTEC) progenitor cells, immature mTECs, mature mTECs, post-AIRE mTECs, tuft cells, neuroendocrine cells, ionocytes, ciliated cells, myelin expressing cells, and/or myoid cells.

The methods can further comprise transferring the population of cells to an air-liquid interface culture system before transplanting.

The methods can further comprise reaggregating the cells to form a reaggregate before transplanting.

The methods can further comprise reducing or eliminating non-epithelial cells from the culture of differentiated TEP cells. The reducing or eliminating can entail enriching for EPCAM+ TEP cells.

In some aspects, the methods comprise adjusting culture conditions or combining cell types in culture to recapitulate a thymic microenvironment.

Recapitulating the thymic microenvironment can comprise culturing TEP cells under conditions sufficient to support survival of lymphatic endothelium cells, vascular endothelium cells, immune cells, mesenchymal cells, pericytes, red blood cells, or combinations thereof.

Recapitulating the thymic microenvironment can comprise culturing TEP cells under conditions sufficient to support differentiation of TECs and subpopulations thereof. Subpopulations of TECs comprise cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

The methods can comprise culturing definitive endodermal (DE) cells in the first medium comprising an activator of retinoic acid receptor (RAR), an activator of BMP signaling, an activator of FGF signaling, and an inhibitor of TGF-β signaling. In some embodiments, the DE cells are first differentiated in an initial cell culture medium comprising an inhibitor of BMP signaling in advance of being transferred to the first cell culture medium comprising the activator of BMP signaling. In some embodiments, an inhibitor of Wnt signaling is introduced into the first cell culture medium.

The methods can comprise culturing anterior foregut endodermal (AFE) cells produced by said culturing of the DE cells, wherein said culturing of the AFE cells is in the first medium comprising an activator of BMP signaling, an activator of FGF signaling, and an inhibitor of TGF-β signaling.

Ventral pharyngeal endodermal (VPE) cells produced by said culturing of the AFE cells can be cultured in the first medium comprising an activator of BMP signaling, an activator of FGF signaling, and an inhibitor of TGF-β signaling to produce TEP cells. In some embodiments, the AFE cell culture medium is substantially similar to the first cell culture medium except that it is substantially free of an activator of RAR signaling.

The methods can be performed starting with cells obtained from pluripotent stem (PS) cells.

The PS cells can be embryonic stem cells, embryonic germ cells, or induced pluripotent stem cells.

The PS cells can be primate pluripotent stem cells (pPS) cells.

The PS cells can be human pluripotent stem (hPS) cells.

Also provided herein are methods for generating TEP cells in vitro comprising culturing a population of cells comprising anterior foregut endodermal (AFE) cells in a first cell culture medium comprising an activator of BMP signaling, an activator of FGF signaling, and an inhibitor of TGF-β signaling to produce TEP cells. In some embodiments, the AFE, cell culture medium is substantially similar to the first cell culture medium except that it is substantially free of at least one of: an activator of retinoic acid receptor signaling, an activator of Wnt signaling, and an inhibitor of hedgehog signaling.

Also provided herein are methods for generating TEP cells in vitro comprising culturing a population of cells comprising definitive endodermal (DE) in a first cell culture medium comprising an activator of BMP signaling, an activator of retinoic acid receptor signaling, an activator of FGF signaling, an inhibitor of TGF-β signaling, and an inhibitor of Wnt signaling to produce a population of cells comprising AFE cells, and culturing the population of AFE cells in a cell culture medium comprising an activator of BMP signaling, an activator of FGF signaling, and an inhibitor of TGF-β, and substantially free of an inhibitor of Wnt signaling to produce TEP cells.

Some embodiments of the methods provided herein further comprise directing development of one or more subpopulation of thymic epithelial cells in the culture by introducing to the culture medium one or more factors associated with WNT signaling, factors associated with BMP signaling, factors associated with TGF beta signaling, factors associated with IGF signaling, factors associated with FGF signaling, factors associated with NOTCH signaling, TNF receptors or their ligands, factors associated with p53 signaling, and/or Toll-like receptors. Factors may be introduced to the culture medium by, e.g., introducing a soluble form of the one or more factors and/or introducing a cell that expresses the one or more factors. As used herein, the term “factors associated with” means factors that initiate, propagate, inhibit, upregulate the expression of, downregulate the expression of, or otherwise modulate the activity of a pathway or a component of a pathway. For example, factors associated with WNT signaling include factors downstream or upstream of WNT whose expression/activity modulate one or more component of the WNT signaling pathway. In some examples, these factors include proteins which increase in expression upon activation of the signaling pathway, where the expression of these factors may be low or undetectable in absence of the activation of the signaling pathway.

In some embodiments, the thymic microenvironment may be assessed or characterized by determining presence of cells expressing one or more factors associated with WNT signaling, factors associated with BMP signaling, factors associated with TGF beta signaling, factors associated with IGF signaling, factors associated with FGF signaling, factors associated with NOTCH signaling, TNF receptors or their ligands, factors associated with p53 signaling, and/or Toll-like receptors.

In some embodiments of the methods described herein, factors associated with WNT signaling comprise WNT5A, WNT6, ROR1, ROR2, RYK, FRZB, RSPO1, RSPO3, SFRP2, and/or SFRP5; factors associated with BMP signaling comprise BMP4, BMP5, and/or FST; factors associated with TGF beta signaling comprise TGFB1, TGFBR2, CXCL12, and/or CCL21; factors associated with IGF signaling comprise IGF1R; factors associated with FGF signaling comprise FGFR2 and/or FGF7/KGF; factors associated with NOTCH signaling comprise NOTCH1, NOTCH2, NOTCH3, HES1, HES6, DLL4, JAG2, JAG1, HES2, HES4, HEY1, NRARP, DLK1, and/or DLK2; TNF receptors comprise RANK/TNFRSF11A, CD40, LTBR, TNFRSF4, TNFRSF9, LTB, and/or CD70; factors associated with p53 signaling comprise PERP, SFN, CTSD, CDKN2A, and/or CDKN2B; and Toll-like receptors comprise TLR1, TLR2, TLR3, TLR4, TLR5, TLR6, and/or TLR10.

In some aspects, the disclosure provides compositions comprising differentiated cells described herein. For example, the disclosure provides compositions comprising a differentiated population of TEP cells produced according to the methods described herein.

The disclosure also provides compositions comprising reaggregated thymic epithelial progenitor (TEP) differentiated from PS cells, wherein the composition further comprises one or more cell type selected from lymphatic endothelium cells, vascular endothelium cells, immune cells, mesenchymal cells, pericytes, red blood cells, or combinations thereof.

The disclosure also provides compositions comprising TECs differentiated from PS cells, wherein the composition further comprises one or more of cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

The disclosure also provides compositions comprising reaggregated thymic epithelial cells (TECs) differentiated from PS cells, wherein the composition further comprises one or more cell type selected from lymphatic endothelium cells, vascular endothelium cells, immune cells, mesenchymal cells, pericytes, red blood cells, or combinations thereof. In some embodiments, the reaggregated compositions comprise subpopulations including one or more of cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

### BRIEF DESCRIPTION OF THE DRAWINGS

FIG. 1 shows a schematic diagram of a process characterizing transcriptomic profile of thymic cells. In FIG. 1, “MACS” stands for magnetic-activated cell sorting and FACS stands for fluorescence-activated cell sorting.

FIG. 2 shows a heatmap showing average expression of soluble factors, extracellular matrix/adhesion molecules, and chemokines in each of 12 stromal clusters identified.

FIG. 3 shows a heatmap showing the expression of marker genes in each immature TEC (“imm TEC”) cluster.

FIG. 4 shows a heatmap showing the expression of newly identified marker genes in each of 9 identified epithelial clusters.

FIG. 5 shows a dot plot depicting the relative level of expression of Notch signaling ligands, receptors, target genes, and inhibitors in epithelial subsets. Relative size of dots depicts percent of cells in each group expressing the indicated gene.

FIG. 6 shows a dot plot depicting the relative level of expression of selected genes associated with p53 signaling. Relative size of dots depicts percent of cells in each group expressing the indicated gene.

FIG. 7 shows dot plots depicting the relative level of expression of selected TNF Superfamily (top panel) and genes associated with Toll-like receptor (bottom panel) signaling. Relative size of dots depicts percent of cells in each group expressing the indicated gene.

FIG. 8 shows a schematic diagram of a process for differentiating pluripotent stem cells to mature thymic epithelial progenitor cells (“mTEP”) in vitro and further to thymic epithelial cells (TECs) in vivo.

### DETAILED DESCRIPTION OF THE DISCLOSURE

Current sources of thymic tissue for therapeutic and research uses have certain technical limitations. Typically, thymic tissue is collected from cadavers and cultured in vitro for, e.g., 12-21 days to remove thymocytes and thereby avoid graft problems such as graft-versus-host disease (GVHD). Tissue thus derived must be tested for sterility and screened for infections and quality, and also must be histologically assessed before transplantation. Other complications arising from cadaver or donor-derived thymus tissue include the limited availability of donors/tissue. For example, in many instances, donor tissue must be from subjects less than 9 months old, must be negative for viruses (e.g., HIV, hepatitis B, hepatitis, C, EBV, and/or CMV), and must be from donor with known family history, including with no primary relatives having autoimmune disease. Still, the risk of GVHD cannot be avoided. Further, matching of HLA types in donor tissue and recipient is almost impossible with current methods. Moreover, current methods have relatively low success rate with regard to culturing donor-derived tissue and/or transplant of donor derived tissues, with approximately 30% failure rate. Causes contributing to the high failure rate are not always known in every case, any may include complications such as infections arising in donor tissue, complications due to other unknown congenital anomalies See, e.g., Markert, M. L., Devlin, B. H., & McCarthy, E. A. (2010). Thymus transplantation. Clinical Immunology (Orlando, Fla.), 135(2), 236-246, incorporated by reference herein in its entirety. Thus, novel sources of human thymus tissue, including thymic epithelial progenitor (TEP) cells and thymic epithelial cells (TECs), and novel methods of preparing TEPs and TECs, including in vitro methods which yield thymic cells for human transplant and other therapeutic applications, are needed.

Improved methods of generating TEPs, including generating TEPs from stem cells such as pluripotent stem (PS) cells, and generating TEPs in vitro, are provided herein.

Also provided are methods of generating thymic cell populations comprising TEPs and supporting cells that recapitulate the complexity of an endogenous thymic microenvironment in vitro.

Improved methods of generating TECs, including generating TECs from stem cells such as pluripotent stem (PS) cells, and generating TECs in vitro, are provided herein.

Also provided are methods of generating thymic cell populations comprising TECs and supporting cells that recapitulate the complexity of an endogenous thymic microenvironment in vitro.

In some embodiments, the TEPs are reaggregated into three-dimensional structures that recapitulate the complexity of an endogenous thymic microenvironment as described herein. See, e.g., Gill, Jason, et al. “Generation of a complete thymic microenvironment by MTS24+ thymic epithelial cells.” Nature Immunology, 3(7), 635-642, incorporated by reference herein in its entirety. See also Park, Jong-Eun, et al. “A cell atlas of human thymic development defines T cell repertoire formation.” Science 367.6480 (2020), incorporated herein by reference in its entirety.

Also provided herein are methods and results of a comprehensive single-cell transcriptomic analysis of human thymic stromal cells with a particular focus on the epithelial compartment. Ionocytes were identified as an additional subset of medullary epithelial cells and transcriptome information is provided for rare subsets, including ciliated and Schwann cells.

A subset of postnatal mesenchymal cells was identified that secretes many factors associated with WNT signaling, including the non-canonical WNT ligand WNT5A as well as molecules that can potentiate the WNT/Ca2+ signaling (e.g., RSPO3 and SFRP2). Given that WNT signaling has been implicated as a critical regulator of FOXN1 expression, thymic cellularity, and migration of the thymus during development, these findings point to this subset of mesenchymal cells as an important regulator of these processes. Notably, this population also expressed other critical regulators of epithelial proliferation and differentiation (e.g., BMP4, IGF1, FGF7, and FGF10).

Activin A signaling is critical for TEC maturation while follistatin, an inhibitor of this pathway, contributes to the accumulation of TEPCs by blocking differentiation of TECs. The present findings identify pericytes as the main source of activin A. The findings further reveal that myoid cells express high levels of follistatin, thus providing insight into why conditions such as myasthenia gravis, which affect myoid cell numbers, perturb human TEC differentiation.

Strikingly, CFTR+ ionocytes were identified as an additional subset of epithelial cells found in the human thymic medulla. While ionocytes have been described in lung epithelium, their presence has not been previously reported in the thymus. Intriguingly, pulmonary ionocytes arise from basal cells which also give rise to neuroendocrine and tuft cells. Given that these cell types are also present in the human thymus and that they were found in close proximity to each other in the medulla, with many subsets associated with Hassall's corpuscles, it raises the possibility that a similar progenitor exists in the thymus. This hypothesis is compatible with reports of thymomas comprising neuroendocrine differentiation and thymic carcinomas containing tumor cells with a neuroendocrine phenotype. It is also intriguing that myoid cells occur in different thymic tumors, including different types of histologic variants of thymoma and thymic carcinomas. Tumors showing both rhabdomyoid and epithelial differentiation can also arise in the thymus, suggesting that there might be a common precursor that can give rise to both epithelial and myoid cells. Present transcriptome data showing a branching point between neuroendocrine and myoid cells as well as co-expression of epithelial and myoid markers by immunofluorescence in fetal tissue support this idea.

A role for Notch signaling in the development of different thymic epithelial subsets was identified. While Notch signaling has been extensively studied in the context of T cell commitment and epithelial differentiation in other tissues, a role for this pathway in TEC specification has only been recently reported. In neuronal and muscle stem cells, high and sustained levels of HES1 can inhibit cell differentiation by antagonizing master regulators of cell fate like the proneural factor ACL1 and regulator of myogenesis MYOD1. In contrast, when HES1 expression oscillates, it activates stem cell proliferation by driving oscillations in ASCL1 and MYOD1 expression through periodical repression cycles. Alternatively, terminal differentiation is promoted when expression of ASCL1 or MYOD1 is sustained while HES1 expression and/or activity is inhibited. This mechanism is compatible with the present observation that HES6, a HES1 inhibitor, is highly expressed in neuroendocrine and myoid cells. It is thus possible that HES6-mediated inhibition of HES1 allows stable expression of ASCL1 and MYOD1 in progenitor cells that will eventually differentiate into ASCL1+ neuroendocrine or MYOD1+ myoid cells. Although the mechanisms that lead to high HES1 expression in progenitor cells are not clear, other signaling pathways like BMP have been shown to play an important role in regulating quiescence by upregulating HES1 in neural stem cells or promoting the degradation of ASCL1 in hippocampal stem cells. Since there is evidence that BMP signaling promotes maintenance of thymic progenitors, it is possible that this pathway helps establish and maintain quiescence in TEPCs by upregulating HES1 to a level where it constantly suppresses expression of differentiation factors like ASCL1.

Finally, the present findings provide a valuable database of genes expressed in TECs that will allow a better understanding of promiscuous gene expression in the human thymus. For example, a significant subset of APS-1 antigens was present in AIRE+ mTECs, supporting the idea that these genes are AIRE-dependent in humans. Interestingly, the expression of some of the autoantigens that were not found in AIRE+ cells (CYP21A2, CYP17A1, CYP11A1) are often targeted by autoantibodies in thymoma patients but do not correlate with AIRE expression in thymoma samples, implying that they are not AIRE-dependent genes. In addition to AIRE+ mTECs, other cell types most likely participate in induction of tolerance by providing antigens that can be presented by antigen presenting cells like dendritic cells. For example, myoid cells likely participate in the induction of immune tolerance to muscle antigens. Indeed, many thymoma patients who typically lack myoid cells develop myasthenia gravis (MG), an autoimmune disease of the neuromuscular junction characterized by autoantibodies to the acetylcholine receptor (AChR) or other muscle antigens like titin (TTN). APS-I patients also typically do not have detectable autoantibodies against either AChR or TTN, suggesting that the expression of these antigens is not entirely AIRE-dependent. Importantly, expression of AChR and TTN in the present study was much higher in myoid cells compared to AIRE+ mTECs, supporting the idea that myoid cells are the main source of muscle antigens in the human thymic medulla. This analysis thus provides additional information on the regulation of disease relevant TSAs in the human thymus.

### Definitions

By “pluripotent stem cell” or “pluripotent cell” it is meant a cell that has the ability under appropriate conditions of producing progeny of several different cell types that are derivatives of all of the three germinal layers (endoderm, mesoderm, and ectoderm). Pluripotent stem cells are capable of forming teratomas. Examples of pluripotent stem cells are embryonic stem (ES) cells, embryonic germ stem (EG) cells, induced pluripotent stem (iPS) cells, and adult stem cells. PS cells may be from any organism of interest, including, primate, e.g., human; canine; feline; murine; equine; porcine; avian; camel; bovine; ovine, and so on.

By “embryonic stem cell” or “ES cell” it is meant a cell that a) can self-renew, b) can differentiate to produce all types of cells in an organism, and c) is derived from a developing organism or is an established ES cell line which was derived from a developing organism. ES cells may be derived from the inner cell mass of the blastula of a developing organism. ES cells may be derived from a blastomere generated by single blastomere biopsy (SBB) involving removal of a single blastomere from the eight-cell stage of a developing organism. In general, SBB provides a non-destructive alternative to inner cell mass isolation. SBB and generation of hES cells from the biopsied blastomere is described in Cell Stem Cell, 2008 Feb. 7; 2(2): 113-17. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. Nos. 7,029,913, 5,843,780, and 6,200,806, the disclosures of which are incorporated herein by reference.

By “embryonic germ stem cell”, embryonic germ cell” or “EG cell” it is meant a cell that a) can self-renew, b) can differentiate to produce all types of cells in an organism, and c) is derived from germ cells and germ cell progenitors, e.g., primordial germ cells, i.e., those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al, (1992) Cell 70:841; Shamblott, M., et al. (2001) Proc. Natl. Acad. Sci. USA 98: 113; Shamblott, M., et al. (1998) Proc. Natl. Acad. Sci. USA, 95: 13726; and Koshimizu, U., et al. (1996) Development, 122: 1235, the disclosures of which are incorporated herein by reference.

By “induced pluripotent stem cell” or “iPS cell” it is meant a cell that a) can self-renew, b) can differentiate to produce all types of cells in an organism, and c) is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. iPS cells may be generated by providing the cell with “reprogramming factors”, i.e., one or more, e.g., a cocktail, of biologically active factors that act on a cell to alter transcription, thereby reprogramming a cell to pluripotency. Examples of methods of generating and characterizing iPS cells may be found in, for example, Application Nos. US20090047263, US20090068742, US20090191159, US20090227032, US20090246875, and US20090304646, the disclosures of which are incorporated herein by reference.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to self-renew and naturally give rise to all or some cell types of the central nervous system but cannot give rise to cells of the mesoderm or endoderm lineages.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time).

By “endoderm” it is meant the germ layer formed during animal embryo genesis that gives rise to the gastrointestinal tract, respiratory tract, endocrine glands and organs, certain structures of the auditory system, and certain structures of the urinary system.

By “mesoderm” it is meant the germ layer formed during animal embryogenesis that gives rise to muscles, cartilage, bones, dermis, the reproductive system, adipose tissue, connective tissues of the gut, peritoneum, certain structures of the urinary system, mesothelium, notochord, and spleen.

By “ectoderm” it is meant the germ layer formed during animal embryogenesis that gives rise to the nervous system, tooth enamel, epidermis, hair, nails, and linings of mucosal tissues.

By “bone morphogenic proteins” or “BMPs” it is meant the family of growth factors that is a subfamily of the transforming growth factor (3 (TGF superfamily. BMPs (e.g. BMP1, BMP2, BMP3, BMP4, BMP5, BMP6, BMP7, BMP8a, BMP8b, BMP9/GDF, BMP10, BMP11/GDF11, BMP12/GDF7, BMP13/GDF6, BMP14/GDF5, BMP15/GDF9B) were first discovered by their ability to induce the formation of bone and cartilage. BMPs interact with specific receptors on the cell surface, referred to as bone morphogenetic protein receptors (BMPRs). Signal transduction through BMPRs results in mobilization of members of the SMAD family of proteins, which in turn modulate transcription of target genes. Of particular interest in the present invention are activators of BMP signaling, which can readily be identified by one of ordinary skill in the art by any of a number of methods, for example competitive binding assays for binding to BMP or BMP receptors, functional assays, e.g., measuring enhancement of activity of downstream signaling proteins such as relocalization of SMADs, such as, BR-Smad to the nucleus and transcriptional activation of downstream gene targets as known in the art.

By “transforming growth factor betas”, “TGF-s”, and “TGFBs” it is meant the TGFB secreted proteins belonging to the subfamily of the transforming growth factor β (TGF) superfamily TGFBs (TGFB1, TGFB2, TGFB3) are multifunctional peptides that regulate proliferation, differentiation, adhesion, and migration and in many cell types. The mature peptides may be found as homodimers or as heterodimers with other TGFB family members. TGFBs interact with transforming growth factor beta receptors (TGF-Rs, or TGFBRs) on the cell surface, which binding activates MAP kinase-, Akt-, Rho- and Rac/cdc42-directed signal transduction pathways, the reorganization of the cellular architecture and nuclear localization of SMAD proteins, and the modulation of target gene transcription. Of particular interest in the present invention are inhibitors of TGFB signaling, which can be readily identified by one of ordinary skill in the art by any of a number of methods, for example competitive binding assays for binding to TGFB or TGFB receptors, or functional assays, e.g., measuring suppression of activity of downstream signaling proteins such as MAPK, Akt, Rho, Rac, and SMADs, e.g., AR-Smad, etc., as well known in the art.

By “Wnts” it is meant the family of highly conserved secreted signaling molecules which play key roles in both embryogenesis and mature tissues. The human Wnt gene family has at least 19 members (Wnt-1, Wnt-2, Wnt-2B/Wnt-13, Wnt-3, Wnt3a, Wnt-4, Wnt-5A, Wnt-5B, Wnt-6, Wnt-7A, Wnt-7B, Wnt-8A, Wnt-8B, Wnt-9A/Wnt-14, Wnt-9B/Wnt-15, Wnt-10A, Wnt-10B, Wnt-11, Wnt-16). Wnt proteins modulate cell activity by binding to Wnt receptor complexes that include a polypeptide from the Frizzled (Fz) family of proteins and a polypeptide of the low-density lipoprotein receptor (LDLR)-related protein (LRP) family of proteins. Once activated by Wnt binding, the Wnt receptor complex will activate one or more intracellular signaling cascades. These include the canonical Wnt signaling pathway; the Wnt/planar cell polarity (Wnt/PCP) pathway; and the Wnt-calcium (Wnt/Ca2+) pathway.

By culturing under “non-adherent conditions” it is meant culturing under conditions that suppress the adhesion of cells to the vessel in which they are cultured, e.g., the bottom of a tissue culture plate or flask. In some instances, the cells are naturally non-adherent, i.e., they will not adhere to a surface unless the surface is coated with a matrix composition, e.g., fibronectin, laminin, poly-ornithin, poly-lysine, collagen IV, a cell culture medium such as Matrigel, and polycarbonate membranes. In some instances, cells may be maintained in a non-adherent state by agitating the culture. Cells cultured in suspension conditions, for example, may be cultured under non-adherent conditions.

By culturing under “adherent conditions” it is meant culturing under conditions that promote the adhesion of cells to the container in which they are cultured, e.g., the bottom of a tissue culture plate or flask. In some instances, cells may be induced to adhere to the container simply by keeping the culture stationary. In some instances, the wall of the container to which it is desirable to promote adhesion may be coated with a composition to which the cells may adhere, e.g., fibronectin, laminin, poly-ornithine, poly-lysine, collagen IV, a cell culture medium such as Matrigel, and polycarbonate membranes.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual”, “subject”, “host”, and “patient” are used interchangeably herein and refer to any mammalian subject for whom diagnosis, treatment, or therapy is desired, particularly humans.

The terms “medium” in context of cell culture or the phrase “cell culture medium” or “cell medium” refer to a cellular growth medium suitable for culturing of cells of the disclosure, for e.g., PS cells, DE cells, AFE cells, VPE cells, TEP cells. Examples of cell culture medium include Minimum Essential Medium (MEM), Eagle's Medium, Dulbecco's Modified Eagle Medium (DMEM), Dulbecco's Modified Eagle Medium: Nutrient Mixture F-12 (DMEM/F12), F10 Nutrient Mixture, Ham's F10 Nutrient Mix, Ham's F12 Nutrient Mixture, Medium 199, RPMI, RPMI 1640, reduced serum medium, basal medium (BME), DMEM/F12 (1:1), and the like, and combinations thereof. The medium or cell culture medium may be modified by adding one or more additives. Additives may include serum, such as, fetal bovine serum and/or serum replacement agents, such as, B27, N2, KSR, and combinations thereof, and differentiation factors, such as, activators of RA receptor, nodal, Act-A, Act-B, Wnt family members, activators of BMP signaling, inhibitors of TGF-β signaling, FGF, inhibitors of hedgehog signaling, and the like, and combinations thereof.

The term “isolated” in context of cells or cell population refers to cells that are in an environment other than their native environment, such as, apart from tissue of an organism.

The phrase “differentiation factors” as used herein refers to the agents that are included in the medium for culturing cells of the present disclosure, which agents promote the differentiation of the cells from a first cell type to a second cell type.

As used herein, “expression” and grammatical equivalents thereof, in the context of a marker, refers to production of the marker as well as level or amount of the marker. For example, expression of a marker or presence of a marker in a cell or a cell is positive for a marker, refers to expression of the marker at a level that is similar to a positive control level. The positive control level may be determined by the level of the marker expressed by a cell known to have the cell fate associated with the marker. Similarly, absence of expression of a marker or a cell is negative for a marker, refers to expression of the marker at a level that is similar to a negative control level. The negative control level may be determined by the level of the marker expressed by a cell known to not have the cell fate associated with the marker. As such, absence of a marker does not simply imply an undetectable level of expression of the marker, in certain cases, a cell may express the marker but the expression may be low compared to a positive control or may be at a level similar to that of a negative control.

As used herein, “marker” refers to any molecule that can be measured or detected. For example, a marker can include, without limitations, a nucleic acid, such as, a transcript of a gene, a polypeptide product of a gene, a glycoprotein, a carbohydrate, a glycolipid, a lipid, a lipoprotein, a carbohydrate, or a small molecule (for example, a molecule having a molecular weight of less than 10,000 amu).

A “variant” polypeptide means a biologically active polypeptide as defined below having at least 70%, 75%, 80%, 85%, 90%, 95%, 98%, or 99% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, at least about 95%, or at least about 99%. The variant polypeptides can be naturally or non-naturally glycosylated, i.e., the polypeptide has a glycosylation pattern that differs from the glycosylation pattern found in the corresponding naturally occurring protein. The variant polypeptides can have post-translational modifications not found on the natural polypeptide.

As used herein, “analog” or “functional analog” in the context of a molecule, such as a ligand, a peptide, a polypeptide, or the like, refers to a molecule having similar functional properties but a different structure compared to the naturally occurring form of that molecule. In certain cases, the functional analog may be a small molecule that, for example, exhibits the function of a polypeptide. Any functional analog of the differentiation factors disclosed herein may be used in the methods and may be present in the compositions described herein. Such functional analogs are described in the literature and can also be identified by screening of library of compounds, such as, combinatorial compound libraries, peptide libraries, and the like.

The terms “enriching” or “enriched” are used interchangeably herein and mean that the yield (fraction) of cells of one type is increased by at least 10% over the fraction of cells of that type in the starting culture or preparation.

The terms “thymic microenvironment” and “endogenous thymic microenvironment” and the like as used herein refer to the heterogeneous nature of cell types and subpopulations of cells, including extracellular factors and, in some cases, supercellular structures, e.g., organoids, that are found in endogenous thymic tissue, including fetal thymic tissue, post-natal thymic tissue, and/or adult thymic tissue.

As used herein, the phrase “substantially free of” and grammatical equivalents thereof in the context of a supplement/factor in a cell culture medium means that a recited supplement or factor is not present in an amount that would have an effect on differentiation of the cells present in the cell culture medium. Thus, this phrase allows for presence of undetectable or trace amounts of the supplement/factor. For example, a medium substantially free of an activator of RAR may include trace amounts of the activator, e.g., less than 0.2 μM, less than 0.1 μM, less than 0.02 μM, less than 0.01 μM, less than 0.002 μM, or less than 0.001 μM.

### Generating Thymic Epithelial Cells In Vitro

Improved methods and compositions for generating thymic epithelial progenitor (TEP) cells are provided. Also provided are improved methods and compositions for generating thymic epithelial cells (TECs). The methods may comprise culturing a population of cells under culture conditions sufficient to differentiate the cells to TECs. In some embodiments, the methods are performed in vitro. Methods and compositions described in Bautista, J. L., et al. may be useful in the present disclosure (Bautista, J. L., et al. Nat Commun 12, 1096 (2021) (doi.org/10.1038/s41467-021-21346-6; the contents of which are herein incorporated by reference in its entirety).

The methods described herein provide for generating TECs from, for example, a population of cells comprising thymic epithelial progenitor (TEP) cells. Thus, some embodiments entail differentiating TEP cells to TECs in vitro. In some embodiments, the population of cells differentiated into TECs is derived from pluripotent stem (PS) cells. The PS cells can be primate PS cells, including human PS cells. In some embodiments, the PS cells are or are derived from embryonic stem cells, embryonic germ cells, or induced pluripotent stem cells.

The methods of culturing a population of cells under culture conditions sufficient to differentiate the cells to TECs may comprise culturing the population of cells in a medium comprising an activator of bone morphogenetic protein (BMP) signaling, an activator of fibroblast growth factor (FGF) signaling, and an inhibitor of transforming growth factor-β (TGF-β) signaling, as described herein.

The methods of culturing a population of cells under culture conditions sufficient to differentiate the cells to TECs may comprise culturing the population of cells under the conditions sufficient to differentiate the cells to TECs for 1 day, for 2 days, for 3 days, for 4 days, for 5 days, for 6 days, or for longer than 6 days.

The methods of culturing a population of cells may further comprise culturing in a medium comprising one or more of keratinocyte growth factor (KGF), heparin, hydrocortisone, and/or a triiodo-L-thyronine (T3) supplement, or an analog thereof. As used herein, the term “KGF” refers to FGF7 and functionally active fragments thereof. As used herein, the term “heparin” refers to heparin as well as analogs thereof. As used herein, the term “hydrocortisone” refers to hydrocortisone and analogs thereof. As used herein, the term “T3 supplement” refers to T3 and analogs thereof. Analogs include derivatives as well as structurally similar or structurally dissimilar but functionally similar molecules. In certain examples, an analog may have an increased potency and/or increased stability as compared to the reference molecule.

The method of culturing a population of cells may comprise culturing the population of cells under the conditions sufficient to differentiate the cells to TECs for 1 day, for 2 days, for 3 days, for 4 days, for 5 days, for 6 days, for 7 day, for 8 days, for 9 days, for 10 days, for 11 days, for 12 days, for 13 days, or for longer than 13 days.

In some embodiments, culturing the population of cells under culture conditions sufficient to differentiate the cells to TECs comprises culturing the population of cells in a medium comprising one or more of an insulin-transferrin-selenium (ITS) supplement and/or a B27 supplement.

The methods described herein for generating TECs may comprise culturing a population of cells in a liquid medium. Some embodiments provide for methods of culturing a population of cells to generate TECs in suspension culture, such that the conditions sufficient to differentiate TECs in vitro comprise use of suspension culture conditions.

The methods may be performed using a minimum essential medium (MEM) or Dulbecco's minimum essential medium (DMEM).

In some embodiments, the methods of culturing a population of cells under culture conditions sufficient to differentiate the cells to TECs produces a differentiated population of cells comprising subpopulations of TECs. The subpopulations of TECs may comprise one or more of cTEC lineage cells, bipotent TEP cells, committed mTEC progenitor cells, immature mTECs, mature mTECs, post-AIRE mTECs, tuft cells, neuroendocrine cells, and/or myoid cells.

Some embodiments of the methods described herein comprise transferring the cells to a cell culture medium such as Matrigel or to an air-liquid interface culture system. The cells may be transferred to the cell culture medium such as Matrigel or the air-liquid interface culture system at any stage of differentiation.

Some embodiments of the methods described herein may further comprise reaggregating the cells to form a reaggregate.

Also provided are methods for generating thymic epithelial cells (TECs) comprising culturing thymic epithelial progenitor (TEP) cells in suspension culture, optionally transferring the TEP cells to an air-liquid interface culture system, and differentiating the TEP cells to TECs. In some embodiments, the methods may further comprise transplanting the TEP cells to a subject, wherein the TEP cells differentiate to TECs in vivo. The methods may comprise transplanting the TEC cells, with or without TEP cells, to a subject, wherein the TEP cells differentiate to TECs in vivo.

The TEP cells and/or TECs may be reaggregated to form a reaggregate. In some embodiments, the reaggregate is transplanted to the subject. In some embodiments, the transplanted reaggregate comprises TEP cells that differentiate into TECs in vivo. In some embodiments, the transplanted reaggregate comprises TECs prior to transplantation. In some embodiments, the transplanted reaggregate comprises subpopulations of TECs prior to transplantation.

The methods described herein may further comprise reducing or eliminating non-epithelial cells from the culture. In some embodiments, the cultured cells are enriched for EPCAM+ cells. In some embodiments, the cultured cells are enriched for EPCAM+ TEP cells. In some embodiments, the cultured cells are enriched for EPCAM+ TECs.

Some embodiments of the methods described herein comprise adjusting culture conditions to recapitulate a thymic microenvironment. Some embodiments of the methods described herein comprise combining cell types in culture to recapitulate a thymic microenvironment. A thymic microenvironment may be a complex system of cell types similar to an endogenous thymic microenvironment, e.g., in fetal, post-natal, or adult thymic tissue as described herein.

In some embodiments, recapitulating a thymic microenvironment comprises culturing TEP cells under conditions sufficient to support survival of lymphatic endothelium cells, vascular endothelium cells, immune cells, mesenchymal cells, pericytes, red blood cells, or combinations thereof.

In some embodiments, recapitulating a thymic microenvironment comprises culturing TEP cells under conditions sufficient to support differentiation of TECs and subpopulations thereof. Subpopulations of TECs may comprise one or more of cTEC lineage cells, bipotent TEP cells, committed mTEC progenitor cells, immature mTECs, mature mTECs, post-AIRE mTECs, tuft cells, neuroendocrine cells, and/or myoid cells.

In some embodiments, definitive endodermal (DE) cells are cultured in a medium comprising an activator of retinoic acid receptor, an activator of bone morphogenetic protein (BMP) signaling, an activator of fibroblast growth factor (FGF) signaling, and an inhibitor of transforming growth factor-β (TGF-β) signaling. An inhibitor of BMP signaling may be introduced into the medium in advance of the activator of BMP signaling. An inhibitor of Wnt signaling may be introduced into the medium.

In some embodiments, anterior foregut endodermal (AFE) cells are produced by culturing of DE cells in a medium comprising an activator of retinoic acid receptor, an activator of bone morphogenetic protein (BMP) signaling, an inhibitor of transforming growth factor-β (TGF-β) signaling, and an inhibitor of Wnt signaling.

In some embodiments, ventral pharyngeal endodermal (VPE) cells are produced by culturing of AFE cells in a medium comprising an activator of retinoic acid receptor and an activator of bone morphogenetic protein (BMP) signaling.

The starting cells of the methods described herein may be or may be obtained from PS cells, including, for example, embryonic stem cells, embryonic germ cells, or induced pluripotent stem cells. The PS cells may be, for example, primate pluripotent stem cells (pPS) cells. The PS cells may be human pluripotent stem (hPS) cells.

### Method of Generating Thymic Epithelial Progenitor Cells In Vitro

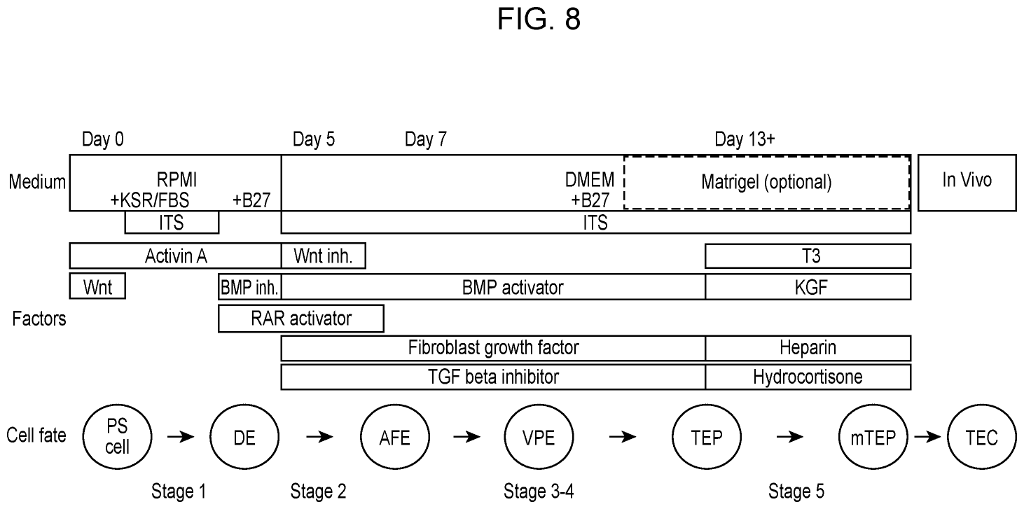

FIG. 8 shows a schematic of an exemplary process for differentiating human stem cells to TEP cells in vitro, e.g., cultured in suspension. The process can further include differentiating to TECs in vivo. In a particular embodiment of the process schematized in FIG. 8, TEP cells are generated using a MEL1-FOXN1-GFP reporter cell line. In some embodiments, production of TEP cells from PS cells involves four stages of differentiation:

Stage 1: Culturing of PS cells under conditions suitable to produce DE cells

Stage 2: Culturing of DE cells under conditions suitable to produce AFE, cells

Stage 3: Culturing of AFE cells under conditions suitable to produce VPE cells

Stage 4: Culturing of VPE cells under conditions suitable to produce TEP cells

At least stages 1 through 3 are performed in cell suspension conditions. In some embodiments, Stages 1 through 4 are performed in cell suspension conditions. Culturing TEPs cells in suspension, or performing in suspension culture at least part of the process of culturing TEPs cells, allows for scale-up of TEP cell and TEC preparation. In some embodiments, the methods provided herein comprise generating thymic epithelial cells (TECs) by culturing thymic epithelial progenitor (TEP) cells entirely in suspension, including differentiating the TEP cells in suspension. The methods can further comprise transplanting TEP cells into a subject, e.g., a human subject, wherein the TEP cells may further differentiate into TECs in vivo.

In some embodiments, following Stage 3, VPE cells may be cultured on a cell culture medium such as Matrigel under conditions suitable to produce TEPs. In other embodiments, TEP cells and/or TECs may be reaggregated, for example, from suspension culture. Cells can be enriched for EPCAM+ cells, thereby depleting non-epithelial cells. Enriched EPCAM+ cells can be reaggregated with other support cell types including e.g., fibroblasts, endothelial cells, pericytes, and immune cells. Thus, thymic epithelial cells recapitulating the complexity of the endogenous thymic microenvironment are produced in vitro according to the methods provided herein. In some embodiments, TEP cells and/or TECs are cultured under conditions sufficient to establish various subsets of TECs in a population of cells, including for example the subpopulations described in Table 1. In some embodiments, the methods of generating TEP cells and/or TECs described herein give rise to populations of thymic epithelial cells having diverse subpopulations of epithelial cells similar to those found in human fetal, post-natal, or adult thymic tissue. For example, the methods of generating TEP cells and/or TECs described herein give rise to populations of thymic epithelial cells having subpopulations representing one or more of the subpopulations of thymic epithelial cells shown in Table 1.

The methods may further comprise reaggregating the TEP cells and transplanting the reaggregates into a subject. In some embodiments, transplanting reaggregated TEP cells into a subject further comprises differentiating the TEP cells to TECs in vivo.

Culturing TEP cells in cell suspension culture, in whole or in part, as described herein may avoid the use of a two-dimensional cell culture system(s). Culturing TEP cells in cell suspension culture, in whole or in part, has the added benefit of facilitating in vitro analysis and characterization of TEP cells as well as cells at various other stages of development toward TEP cells. For example, the use of cell suspension culture allows for easy removal of cell samples at various timepoints in the protocol and facilitates subsequent analysis using, e.g., flow cytometry and other fluidics-based cytometric analytical and sorting approaches, including, e.g., fluorescence-activated cell sorting (FACS).

In some embodiments, Stages 1 through 4 are accomplished in 12 days or less, and generation of TECs enriched for EPCAM+ cells and reaggregated with other cell types are produced in 35 days or less, 34 days or less, 33 days or less, 32 days or less, 31 days or less, 30 days or less, 29 days or less, 28 days or less, 27 days or less, 26 days or less, or 26 days or less.

Culturing at each stage is conducted under culture conditions and for a time sufficient to produce the product of that stage, where the product may be characterized by expression of one or more markers and/or by functional characterization as described in more detail below. The culture medium of each of these stages is described below.

The methods of the present disclosure contemplate methods that begin at Stage 1, Stage 2, Stage 3, or Stage 4.

### Stage 1: Culturing of PS Cells to Produce DE Cells

As noted above, a method for generating thymic epithelial progenitor (TEP) cells from PS cells in vitro is provided.

In certain embodiments, the method includes differentiation of PS cells into DE cells. PS cells may be differentiated into DE cells by culturing the pluripotent stem cells in a medium comprising a growth factor, which can be one or more of Nodal, Activin A, and Activin B, or variants or analogs thereof. In certain cases, the medium for culturing the PS cells for inducing differentiation into DE cells may include a combination of Activin A and Activin B.

In certain cases, the medium for culturing the PS cells for inducing differentiation into DE cells may include one or more of Nodal, Activin A, Activin B in combination with an activator of BMP signaling. In certain cases, the medium for inducing differentiation of PS cells into DE cells may include one or both of Activin A and Activin B in combination with an activator of BMP signaling, but the medium for inducing differentiation of PS cells into DE cells does not require an activator of BMP signaling. Hence, some embodiments of the methods of inducing differentiation of PS cells into DE cells utilize a medium that does not comprise an activator of BMP signaling.

In certain cases, the medium for inducing differentiation of PS cells into DE cells may include one or more of Nodal, Activin A, Activin B, an activator of BMP signaling, and a Wnt family member.

PS cells may be cultured in a differentiation medium that includes one or more of Nodal, Activin A, Activin B, an activator of BMP signaling, and a Wnt family member for a period of 1 day to 5 days, thereby generating DE cells.

In certain cases, PS cells may be cultured to produce DE cells in a differentiation medium that includes Activin A. In certain cases, PS cells may be cultured to produce DE cells in a differentiation medium that includes Activin A and Activin B. In certain cases, PS cells may be cultured to produce DE cells in a differentiation medium that includes Activin A, Activin B, and BMP4. The culturing may be carried out for 1 day to 6 days. In certain cases, the DE cells are generated from PS cells as described in U.S. Pat. No. 8,216,836, which is herein incorporated by reference in its entirety.

In certain cases, DE cells may be obtained from PS cells by culturing PS cells for a period of 1 day to 6 days or more in a medium that includes one or more of Nodal, Activin A, Activin B. In certain cases, the culturing of the PS cells in the medium that includes one or more of Nodal, Activin A, Activin B may be carried out for 1 day, 2 days, 3 days, 4 days, 5 days, or 6 days, thereby generating PS cells.

In certain cases, DE cells may be obtained from PS cells by culturing PS cells in a medium that includes one or more of Nodal, Activin A, Activin B in combination with a Wnt family member for a period of 1 day to 5 days, such as, 1 day, 2 days, 3 days, 4 days, 5 days, or 6 days. In certain cases, the PS cells may be cultured in a medium that includes one or more of Nodal, Activin A, Activin B in combination with a Wnt family member for a period of 1 day or 2 days, after which the culturing is carried out in a medium that includes one or more of Nodal, Activin A, Activin B but is substantially free of a Wnt family member. In certain cases, the PS cells may be cultured in a medium that includes one or more of Nodal, Activin A, Activin B in combination with a Wnt family member for a period of 1 day or 2 days, after which the culturing is carried out in a medium that includes one or more of Nodal, Activin A, Activin B but is substantially free of a Wnt family member, where the culturing without the Wnt family member may be carried out for 2 days, after which an activator of retinoic acid receptor may be included in the medium and the culturing carried out for an additional day or two days in the presence of one or more of Nodal, Activin A, Activin B and the activator of retinoic acid receptor.

In certain cases, the DE cells obtained by differentiation of PS cells may express certain markers of DE cells. For example, the DE cells may express one or more of DE cell markers such as SOX17, FOXA2 (also known as HNF3B or HNF3), GSC, M1XL1, and CXCR4. In addition, the DE cells generated by the methods described herein do not express markers of mesoderm cell fate or ectoderm cell fate. As such, the DE cells do not express Brachyury, MOX1, SOX1, or ZIC1. In addition, the DE cells of the method described herein do not express markers of extra-embryonic visceral endoderm. For example, the DE cells disclosed herein do not express visceral endoderm markers, such as, SOX7. In certain cases, the DE cells produced by the methods disclosed herein are positive for expression one or more DE cell markers, such as, SOX17, FOXA2, GSC, M1XL1, and CXCR4 and express no or low levels of AFP, SPARC, thrombomodulin, and SOX7.

In certain cases, DE cells can be obtained by differentiation of PS cells by culturing PS cells in a medium that is supplemented with Insulin-Transferrin-Selenium (ITS) supplement, e.g., ITS-G 100× (Gibco). In such embodiments, ITS can be provided to the culture medium in concentrations ranging from about 1:10 (v/v) to about 1:10,000 (v/v) or in concentrations greater than about 1:200 (v/v). In certain embodiments, the concentration of ITS in the medium is about 1:1000 (v/v), about 1:900 (v/v), about 1:800 (v/v), about 1:700 (v/v), about 1:600 (v/v), about 1:500 (v/v), about 1:400 (v/v), about 1:300 (v/v), about 1:200 (v/v), about 1:100 (v/v), or about 1:50 (v/v).

In certain cases, DE cells can be obtained by differentiation of PS cells by culturing PS cells in a medium that includes an inhibitor of BMP signaling. In certain cases, LDN193189 “LDN” can be used as an inhibitor of BMP signaling.

Some embodiments of the methods of producing DE cells by culturing PS cells comprise an approximately 4-day Stage 1, wherein on day 1 PS cells are cultured in RPMI medium supplemented with about 0.2% KSR/FBS and Activin A and Wnt3a at approximately 100 ng/ml and 50 ng/ml, respectively; on days 2 to 3 PS cells are cultured in RPMI medium supplemented with about 0.2% KSR/FBS and Activin A and ITS at approximately 100 ng/ml and at a dilution of 1:1000, respectively; and on day 4 PS cells are cultured in RPMI medium supplemented with B27 (0.5× or 1:1000 dilution (v/v)) in place of KSR and Activin A, an retinoic acid (or other activator of retinoic acid receptor (RAR)), and LDN (or other inhibitor of BMP signaling) at approximately 100 ng/ml, 0.25 uM, and 250 nM, respectively.

### Stage 2: Culturing of DE Cells to Produce AFE Cells

As noted above, a method for generating thymic epithelial progenitor (TEP) cells in vitro is provided. In certain embodiments, the method includes culturing definitive endodermal (DE) cells obtained from pluripotent stem cells in a medium that includes an activator of retinoic acid receptor, an activator of bone morphogenetic protein (BMP) signaling, an activator of fibroblast growth factor signaling, an inhibitor of Wnt signaling, and an inhibitor of transforming growth factor-β (TGF-β) signaling (i.e., TGF-β RI Kinase Inhibitor IV (TGFbi IV) (Calbiochem)) to produce AFE cells.

The culturing may be carried out for 1 day to 6 days or more. For example, the culturing of DE cells may be carried out for 2-6 days, 1-5 days, 1-3 days, 2-5 days, 2-4 days, 2-3 days, 1 day, 2 days, 3 days, 4 days, 5 days, or 6 days.

In certain embodiments, the medium for culturing DE cells to produce TEP cells may not include Nodal or activins, such as Activin-A (ActA) or Activin-B (ActB).

The AFE, cells produced by the methods described herein may express one or more markers of AFE cells. For example, the AFE, cells produced by the methods described herein may express SOX2, FOXA2 and/or HHEX. In addition, the AFE cells produced by the methods described herein may not express the posterior foregut endoderm marker CDX2.

In some embodiments, AFE cells can be obtained by culturing DE cells in a medium that includes an activator of retinoic acid receptor, an activator of bone morphogenetic protein (BMP) signaling, an activator of fibroblast growth factor signaling, an inhibitor of transforming growth factor-β (TGF-β) signaling (i.e., TGF-β RI Kinase Inhibitor IV (Calbiochem)), an inhibitor of Wnt signaling (i.e., IWP2), and ITS.

Some embodiments of the methods of producing AFE cells by culturing DE cells comprise an approximately 2-day Stage 2, wherein on the first day of Stage 2 DE cells are cultured in a DMEM medium supplemented with B27 at 0.5×, BMP4 or other activator of BMP signaling at about 50 ng/ml, retinoic acid or other activator of RAR at about 0.25 uM, FGF8 or other fibroblast growth factor at about 50 ng/ml, TGFbi IV or other inhibitor of TGFβ signaling at about 2.5 uM, IWP2 or other inhibitor of Wnt signaling at about 5 uM, and ITS at a concentration of about 1:1000.

### Stage 3: Culturing of AFE Cells to Produce VPE Cells

In certain embodiments, the production of TEP cells from DE cells may include an intermediate stage of production of VPE cells from the AFE, cells by the above-mentioned culturing of AFE cells.

As such, VPE cells may be produced by culturing the AFE cells in a medium that contains an activator of RA receptor, an activator of BMP signaling, an inhibitor of TGF-0 signaling, as described above.

Alternatively, VPE cells may be produced by culturing AFE cells in a medium that contains an activator of BMP signaling, an activator of fibroblast growth factor signaling, and an inhibitor of TGF-β signaling. In some embodiments, the medium is substantially free of one or more of: an activator of Wnt signaling or Wnt3a, an inhibitor of hedgehog signaling (e.g., cyclopamine), and an activator of retinoic acid signaling.

The AFE, cells may be cultured in the medium described above for a period of about 1 day to 8 days (e.g., 1-7 days, 1-5 days, 1-3 days, 2-7 days, 2-5 days, 2-4 days, 2-3 days, 1 day, 2 days, 3 days, 4 days, 5 days, 6 days, 7 days, or 8 days) to produce VPE cells.

The VPE cells produced by the methods described herein may express one or more markers of VPE cells, such as, HOXA3, PAX1, or EYA1.

Some embodiments of the methods of producing VPE cells by culturing AFE cells comprise an up-to 4-day Stage 3, wherein AFE cells are cultured in a DMEM medium supplemented with B27 at 0.5×, BMP4 or other activator of BMP signaling at about 50 ng/ml, FGF8 or other fibroblast growth factor at about 50 ng/ml, TGFbi IV or other inhibitor of TGFβ signaling at about 2.5 uM, and ITS at a concentration of about 1:200. In some embodiments, the medium is not supplemented with an activator of Wnt signaling or Wnt3a, with cyclopamine, or with an activator of retinoic acid signaling. Stage 3 can proceed for about 1 to about 4 days.

### Stage 4: Culturing of VPE Cells to Produce TEP Cells

The method of producing TEP cells from DE cells produced from PS cells may further include culturing of VPE cells produced by the culturing of the AFE cells, where the culturing of the VPE cells is in a medium comprising an activator of BMP signaling.

In certain cases, the medium for generating thymic epithelial progenitor (TEP) cells from VPE cells produced by the culturing of the AFE, cells may include an activator of BMP signaling, a fibroblast growth factor (i.e., an activator of fibroblast growth factor signaling), and an inhibitor of TGFβ signaling.

In certain cases, the medium for generating thymic epithelial progenitor (TEP) cells from VPE cells produced by the culturing of the AFE, cells may include an activator of RA receptor, an activator of BMP signaling, a Wnt family member, a fibroblast growth factor, and an inhibitor of hedgehog signaling.

In certain cases, the VPE cells may be cultured in the medium for a period of about 1 day to about 10 days, where the VPE cells differentiate into TEP cells. In certain cases, the VPE cells may be cultured in the medium for 1 day to 10 days (e.g., 1-7 days, 1-5 days, 1-3 days, 2-7 days, 2-5 days, 2-4 days, 2-3 days, 1 day, 2 days, 3 days, 4 days, 5 days, 6 days, 7 days, 8 days, 9 days, or 10 days) to produce TEP cells.

Some embodiments of the methods of producing TEP cells by culturing VPE cells comprise an up-to 4-day Stage 4, wherein VPE cells are cultured in a DMEM medium supplemented with B27 at 0.5×, BMP4 or other activator of BMP signaling at about 50 ng/ml, FGF8 or other activator of fibroblast growth factor signaling at about 50 ng/ml, TGFbi IV or other inhibitor of TGFβ signaling at about 2.5 uM, and ITS at a concentration of about 1:200. Stage 4 can proceed for about 1 to about 4 days.

The TEP cells produced by the methods described herein express markers of TEP cells, which markers are present in TEP cells present in thymus or thymic tissue, such as, adult human thymus or fetal human thymus. For example, TEP cells produced by the methods described herein may express the TEP markers at a level similar to the level expressed by cells in adult or fetal thymus. In certain cases, the TEP cells produced by the methods described herein express one or more of FOXN1, HOXA3, EYA1, and EpCAM. In certain cases, the TEP cells produced by the methods provided herein express FOXN1 and HOXA3. In certain cases, the TEP cells produced by the methods provided herein express FOXN1, HOXA3, PAX1, EpCAM, and EYA1.

As such, a method for producing TEP cells from VPE cells by culturing the VPE cells in a medium containing one or more of an activator of RA receptor, an activator of BMP signaling, a Wnt family member, a fibroblast growth factor, and an inhibitor of hedgehog signaling for a period of about 1 day to 10 days is provided.

In certain embodiments, the TEP cells may be generated within about 15 days (e.g., within 10 days-15 days, within 10 days-14 days, within 10 days-13 days, within 10 days-12 days, within 10 days-11 days, such as within 15 days, 14 days, 13 days, 12 days, 11 days, or 10 days) from the start of the culturing of the PS cells (e.g., pPS, such as, primate iPS cells, primate ES cells, human PS, human iPS cells, human ES cells). In certain embodiments, the method includes culturing the PS cells according to the methods described herein for about 1-5 days, e.g., 4 days-5 days to produce DE cells. In certain embodiments, the method further includes culturing the DE cells (produced from the PS cells) according to the methods described herein, for about 1-3 days e.g., 2-3 days (or till day 4-7, e.g., day 5-7 from the start of the culturing of the PS cells) to produce AFE cells. In certain embodiments, the method further includes culturing the AFE cells (produced from the DE cells) according to the methods described herein, for about 1-3 days e.g., 2-3 days (or till day 6-10, e.g., day 7-9 from the start of the culturing of the PS cells) to produce VPE cells. In certain embodiments, the method further includes culturing the VPE cells (produced from the AFE cells) according to the methods described herein, for about 1-3 days e.g., 2-3 days (or till day 10-15, e.g., day 10-12 or day 10-11 from the start of the culturing of the PS cells) to produce TEP cells.

The culturing methods described herein may be carried out in adherent conditions or in non-adherent conditions (e.g., suspension cultures). In some embodiments, the cell populations disclosed herein are cultured as an adherent culture. In some embodiments, the cell populations disclosed herein are cultured as suspension culture.

The PS cells may be from any source. In certain cases, the PS cell may be embryonic stem cell, embryonic germ cells, and induced pluripotent stem cell. In certain cases, the PS cells may be primate pluripotent stem cells (pPS) cells. In certain cases, the pPS cells may be human pluripotent stem (hPS) cells. In certain cases, the hPS cells may be human embryonic stem (hES) cells. The hPS cells may be induced pluripotent stem (iPS) cells. In certain cases, the PS cell may be an established stem cell line. In certain cases, the PS cell may be an established embryonic stem cell line. In certain cases, the PS cell may be an established embryonic stem cell line, which cell line is derived from a blastomere generated by single blastomere biopsy (SBB) involving removal of a single blastomere from the eight-cell stage of a developing organism. In certain embodiments, the PS cell may be an established stem cell line that does not include PS cells or ES cells produced by disaggregating human embryo or human blastocyst.

As noted above, the cell culture medium may include additives or supplements. In certain cases, the cell culture medium may not include serum. In certain cases, the cell culture medium may not include serum but may include serum replacement, such as KSR or B27. The type of cell culture medium and the additives for the cell culture medium may be different for certain differentiation stages of the cell populations.

In certain embodiments, the medium used for the culturing methods described herein may contain reduced serum or no serum. Serum concentrations can range from about 0.05% (v/v) to about 20% (v/v). For example, in certain embodiments, the serum concentration of the medium can be less than about 0.05%>(v/v), less than about 0.1% (v/v), less than about 0.2% (v/v), less than about 0.3%>(v/v), less than about 0.4% (v/v), less than about 0.5%>(v/v), less than about 0.6% (v/v), less than about 0.7% (v/v), less than about 0.8% (v/v), less than about 0.9% (v/v), less than about 1% (v/v), less than about 2% (v/v), less than about 3% (v/v), less than about 4% (v/v), less than about 5% (v/v), less than about 6% (v/v), less than about 7% (v/v), less than about 8%) (v/v), less than about 9% (v/v), less than about 10% (v/v), less than about 15% (v/v) or less than about 20% (v/v). In some embodiments, the cells are grown without serum. In other embodiments, the medium used for the culturing methods described herein may contain no serum and may contain a serum replacement.

In still other embodiments, the medium used for the culturing methods described herein may contain FBS or knockout serum replacement (KSR). In such embodiments, KSR or FBS can be provided to the culture medium in concentrations ranging from about 0.1% (v/v) to about 20% (v/v) or in concentrations greater than about 20% (v/v). In certain embodiments, the concentration of FBS or KSR in the medium is about 0.1% (v/v), about 0.2% (v/v), about 0.3% (v/v), about 0.4% (v/v), about 0.5% (v/v), about 0.6% (v/v), about 0.7% (v/v), about 0.8% (v/v), about 0.9% (v/v), about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 15% (v/v) or about 20% (v/v).

In still other embodiments, the medium used for the culturing methods described herein may contain Insulin-Transferrin-Selenium (ITS) supplement, e.g., ITS-G (100×, Gibco). In such embodiments, ITS can be provided to the culture medium in concentrations ranging from about 1:10 (v/v) to about 1:10,000 (v/v) or in concentrations greater than about 1:200 (v/v). In certain embodiments, the concentration of ITS in the medium is about 1:1000 (v/v), about 1:900 (v/v), about 1:800 (v/v), about 1:700 (v/v), about 1:600 (v/v), about 1:500 (v/v), about 1:400 (v/v), about 1:300 (v/v), about 1:200 (v/v), about 1:100 (v/v), or about 1:50 (v/v).

In certain cases, RPMI 1640 medium may be used for stages 1 and 2 while DMEM/F12 may be used for stages 3 and 4. In certain cases, RPMI 1640 medium supplemented with increasing concentrations of KSR (0% on day 1 of culturing, 0.2% on day 2-day 3 of culturing, and 2% on day 4 of culturing) or B27 (0.5× or 1:1000 dilution (v/v)) for day 5-day 7 of culturing may be used. In certain cases, DMEM/F12 with B27 (0.5× or 1:1000 dilution (v/v)) may be used for stages 3 and 4 of culturing.

In some embodiments, the methods described herein produce thymic epithelial cells and cell populations recapitulating the complexity of the endogenous thymic microenvironment. In some embodiments, TEP cells are cultured under conditions sufficient to establish various subsets of TECs in a population of cells, including for example the subpopulations described in Table 1. In some embodiments, the methods of generating TEP cells described herein give rise to populations of thymic epithelial cells having diverse subpopulations of epithelial cells similar to those found in human fetal or adult thymic tissue. For example, the methods of generating TEP cells described herein give rise to populations of thymic epithelial cells having subpopulations representing one or more of the subpopulations of thymic epithelial cells shown in Table 1.

FIG. 8 shows a schematic of an exemplary process for differentiating human stem cells to thymic epithelial cells (TECs). In a particular embodiment of the process schematized in FIG. 8, medium and differentiation factors are applied according to the description provided in Table 2. In some embodiments, production of TECs from PS cells involves culturing TEP cells under conditions suitable to produce TEP cells. The TEP cells can be obtained, for example, according to the methods described above, including Stages 1 through 4. In some embodiments, the culturing of TEP cells to obtain TECs involves a fifth stage of differentiation: Stage 5: Culturing of TEP cells under conditions suitable to produce matured TEP (mTEP) cells. The mTEP upon in vivo transplantation may produce TECs.

Stage 5: Culturing of TEP Cells to Produce a Population of mTEP Cells

The method of producing TEP cells from DE cells produced from PS cells may include further culturing of TEP cells to produce further matured TEP cells prior to in vivo differentiation into TECs, where the further culturing of the TEP cells is in a medium comprising ITS, T3, KGF, heparin, and/or hydrocortisone. Thus, Stage 5 of the methods of producing TEP cells provided herein results in a population of TEP cells exhibiting further maturation. For example, during or following Stage 5, the proportion of cells exhibiting molecular and/or morphological characteristics of TEP cells is increased, or molecular markers of TEP cells have an increase in the level of expression of one or more TEP cell markers as described herein. Likewise, during or following Stage 5, the proportion of cells exhibiting molecular and/or morphological characteristics of non-TEP cells is decreased, or molecular markers of non-TEP cells have a decrease in the level of expression. In some embodiments, a population of mTEP cells comprise a higher proportion of cells exhibiting molecular and/or morphological characteristics of TEP cells as compared to the proportion of cells exhibiting molecular and/or morphological characteristics of TEP cells at the end of stage 4. For example, the proportion of cells exhibiting molecular and/or morphological characteristics of TEP cells during or following Stage 5 is 50% or more, 55% or more, 60% or more, 65% or more, 70% or more, 75% or more, 80% or more, 85% or more, 90% or more, 95% or more, 96% or more, 97% or more, 98% or more, or 99% or more as compared to the proportion of cells exhibiting molecular and/or morphological characteristics of TEP cells at the end of stage 4. In some embodiments, a molecular characteristic of the population of mTEP cells produced during or following Stage 5 is an increase in the proportion of cells that express FOXN1. In some embodiments, a molecular characteristic of the population of mTEP cells produced during or following Stage 5 is an increase in the level of expression of FOXN1 as compared to the level of FOXN1 expression in TEP cells produced at the end of stage 4. For example, FOXN1 expression can be increased by about 1×, about 1.5×, about 2×, about 2.5×, about 3×, or more compared with the level of FOXN1 expression in TEP cells during or following Stage 4. In some embodiments, during or following Stage 5, TEP cells express higher levels of KRT5 compared with the level of KRT5 expression in TEP cells during or following Stage 4; i.e., in some embodiments, KRT5 expression can be increased by about 1×, about 1.5×, about 2×, about 2.5×, about 3×, or more compared with the level of FOXN1 expression in TEP cells during or following Stage 4. In some embodiments, during or following Stage 5, TEP cells express higher levels of KRT15 compared with the level of KRT15 expression in TEP cells following Stage 4; i.e., in some embodiments, KRT15 expression can be increased by about 1×, about 1.5×, about 2×, about 2.5×, about 3×, or more compared with the level of KRT15 expression in TEP cells during or following Stage 4. In some embodiments, during or following Stage 5, TEP cells express lower levels of DLL1; i.e., in some embodiments, DLL1 expression can be decreased by about 1/1.5×, about 1/2×, about 1/2.5×, about 1/3× compared with the level of DLL1 expression in TEP cells during or following Stage 4. Thus, Stage 5 results in a population of cells having a higher proportion of TEP cells and having TEP cells exhibiting an increase in molecular or morphological characteristics of TEP cells, and reduced proportion of non-TEP cells and cells exhibiting molecular characteristics of non-TEP cells. Without being bound to a theory, it is believed that stage 5 involves further maturation of TEP cells resulting in the higher expression level of TEP cell markers as well as an increase in the number of cells expressing TEP cell markers.

In some embodiments, the medium used for the further culturing of TEP cells may contain Insulin-Transferrin-Selenium (ITS) supplement, e.g., ITS-G (100×, Gibco). In such embodiments, ITS can be provided to the culture medium in concentrations ranging from about 1:10 (v/v) to about 1:10,000 (v/v) or in concentrations greater than about 1:200 (v/v). In certain embodiments, the concentration of ITS in the medium is about 1:1000 (v/v), about 1:900 (v/v), about 1:800 (v/v), about 1:700 (v/v), about 1:600 (v/v), about 1:500 (v/v), about 1:400 (v/v), about 1:300 (v/v), about 1:200 (v/v), about 1:100 (v/v), or about 1:50 (v/v).

In some embodiments, the medium used for the further culturing of TEP cells may contain Triiodo-L-Thyronine (T3) supplement. In such embodiments, T3 can be provided to the culture medium in concentrations ranging from about 20 nM to about 2,000 nM or in concentrations of about 200 nM. In certain embodiments, the concentration of T3 in the medium is about 50 nM, about 100 nM, about 150 nM, about 200 nM, about 250 nM, about 500 nM, or about 1000 nM.

In some embodiments, the medium used for the further culturing of TEP cells may contain a keratinocyte growth factor (KGF)/FGF7 or other fibroblast growth factor activator including, e.g., FGF10, heparin, and cortisol, e.g., hydrocortisone. KGF can be provided to the culture medium in concentrations ranging from 5 ng/ml to 500 ng/ml. In certain embodiments, the concentration of KGF in the culture medium is about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, or about 100 ng/ml.

Heparin can be provided to the culture medium in concentrations ranging from about 1 ug/ml to about 100 ug/ml. In certain embodiments, the concentration of heparin in the culture medium is about 1 ug/ml, about 2 ug/ml, about 4 ug/ml, about 6 ug/ml, about 8 ug/ml, about 10 ug/ml, about 12 ug/ml, about 14 ug/ml, about 16 ug/ml, about 18 ug/ml, or about 20 ug/ml.

Cortisol, e.g., hydrocortisone, can be provided to the culture medium in concentrations ranging from about 0.05 ug/ml to about 5 ug/ml. In certain embodiments, the concentration of hydrocortisone, e.g., in the culture medium is about 0.1 ug/ml, 0.2 ug/ml, 0.3 ug/ml, 0.4 ug/ml, 05. ug/ml, 0.6 ug/ml, 0.7 ug/ml, 0.8 ug/ml 0.9 ug/ml, or about 1.0 ug/ml.

In certain cases, the TEP cells may be cultured in the medium for a period of about 1 day to about 21 days, where the TEP cells are further differentiated into TECs in vivo. In certain cases, the TEP cells may be cultured in the medium for 1 day to 21 days, e.g., about 1-3 days, about 1-5 days, about 1-7 days, about 1-9 days, about 1-11 days, about 1-13 days, about 1-15 days, about 1-17 days, about 1-19 days, about 1-21 days. In some cases, the TEP cells are cultured in the medium for longer than 21 days.

In some embodiments, the TEP cells, including mTEP cells, produced by the methods described herein are introduced to a subject where they undergo further differentiation to TECs. The TEP cells and/or TECs and subpopulations thereof may be transplanted into a subject in need of TE cells. In certain cases, the TEP cells and/or TECs and subpopulations thereof may be transplanted into a target site in a subject that provides appropriate differentiation conditions for the TEP cells and/or TECs and subpopulations thereof to differentiate into TE cells. Cells may be transplanted by any of a number of standard methods in the art for delivering cells to tissue, e.g., injecting them as a suspension in a suitable buffer (saline, PBS, DMEM, Iscove's medium, etc. or a pharmaceutically acceptable carrier), providing them on a solid support, e.g., a bead, a filter such as a mesh filter, a membrane, etc. In certain cases, the TEP cells and/or TECs and subpopulations thereof may be transplanted into the thymus of a subject. In certain cases, the TEP cells and/or TECs may be transplanted under the kidney capsule of a subject.

The TEPs and/or TECs produced by the methods described herein express markers of TECs, which markers are present in TECs present in thymus or thymic tissue, such as, adult human thymus or fetal human thymus. For example, TECs produced by the methods described herein may express the TEC markers at a level similar to the level expressed by cells in adult or fetal thymus. In certain cases, the TEC cells produced by the methods described herein express one or more of CLDN3 and/or CLDN4 (collectively “CLDN3/4”), MHC class II genes (“MHCII”), K5 (KRT5), K15 (KRT15), ASCL1, AIRE, IVL, K10 (KRT10), GNB3, K8 (KRT8), CHGA, SOX2, and/or MYOG.

In some embodiments, TECs produced by the methods described herein comprise populations of cells comprising genetically distinct sub-populations of thymic epithelial cells including, e.g., cortical thymic epithelial cell (cTEC) lineage cells, bipotent TEP cells, committed medullary thymic epithelial cell (mTEC) progenitors, immature mTECs, mature mTECs, post-AIRE mTECs, tuft cells, neuroendocrine cells, and/or myoid cells.

Table 1 depicts subsets of thymic epithelial cells and corresponding cell-identifying markers. In some embodiments, the methods described herein produce populations of TECs comprising genetically distinct sub-populations of thymic epithelial cells, wherein: cTEC lineage cells express one or more of beta5t (PSMB11), CD205 (LY75), CCL25, and K8; bipotent TEP cells express one or more of K5 and K8; committed mTEC progenitor cells express one or more of CLDN3/4 (at low levels), MHC class II genes (at undetectable to low levels), K5, K15, ASCL1; immature mTECs express one or more of CLDN3/4, MHC class II genes at low levels, K5, K15, and ASCL1; mature mTECs express one or more of CLDN3/4 MHC class II genes at high levels, AIRE, and K5; post-AIRE mTECs express one or more of CLDN3/4, MHC class II genes at low levels, IVL, and K10; tuft cells express one or more of CLDN3/4, MHC class II genes at low levels, GNB3, and K8; neuroendocrine cells express one or more of CLDN3/4, MHC class II genes at undetectable to low levels, CHGA, SOX2, and K8; and myoid cells express one or more of CLDN3/4 at low levels, MHB class II genes at undetectable to low levels, MYOG, and K8.

TABLE 1
Thymic Epithelial Cell Subpopulations

| Thymic Epithelial Cell | |
|---|---|
| Subpopulation | Markers |
| cTEC lineage cells | B5t (PSMB11) |
| | CD205 (LY75) |
| | CCL25 |
| | K8 |
| Bipotent TEPs | K5 |
| | K8 |
| Committed mTEC | CLDN3/4 low |
| progenitors | MHCII negative-to-low |
| | K5 |
| | K15 |
| | ASCL1 |
| Immature mTECs | CLDN3/4 |
| | MHCII low |
| | K5 |
| | K15 |
| | ASCL1 |
| Mature mTECs | CLDN3/4 |
| | MHCII high |
| | AIRE |
| | K5 |
| Post-AIRE mTECs | CLDN3/4 |
| | MHCII low |
| | IVL |
| | K10 |
| Tuft cells | CLDN3/4 |
| | MHCII low |
| | GNB3 |
| | K8 |
| Neuroendocrine cells | CLDN3/4 |
| | MHCII negative-to-low |
| | CHGA |
| | SOX2 |
| | K8 |
| Myoid cells | CLDN3/4 low |
| | MHCII negative-to-low |
| | MYOG |
| | K8 |

Some embodiments of the methods of producing TECs by culturing TEP cells comprise a multi-day stage 5, wherein TEP cells are cultured in a DMEM culture medium supplemented with B27 at 0.5× and KGF at 50 ng/ml, ITS at 1:200, heparin at 10 ug/ml, hydrocortisone at 0.5 ng/ml, and T3 at 200 nM.

Table 2 provides an example cell culture protocol for one embodiment of the methods for generating thymic epithelial cells in vitro described herein.

TABLE 2
Culture conditions for generating TEPs in vitro

| | | | | Factor/ | |
|---|---|---|---|---|---|
| | Approxi- | | | Supple- | |
| | mate | | Factors & | ment | |
| | Culture | | Supple- | Concen- | |
| Stage | Day | Medium | ments | tration | |
| 1 | 1 | RPMI + 0.2% | Activin A | 100 | ng/ml |
| | | KSR/FBS | Wnt3a | 50 | ng/ml |
| | 2-3 | RPMI + 0.2% | Activin A | 100 | ng/ml |
| | | KSR/FBS | ITS | 1:1000 | |
| | 4 | RPMI + B27 | Activin A | 100 | ng/ml |
| | | 0.5X | Retinoic | 0.25 | uM |
| | | | acid | | |
| | | | LDN | 250 | nM |
| 2 | 5-6 | DMEM + B27 | BMP4 | 50 | ng/ml |
| | | 0.5X | Retinoic | 0.25 | uM |
| | | | acid | | |
| | | | FGF8 | 50 | ng/ml |
| | | | TGFbi IV | 2.5 | uM |
| | | | IWP2 | 5 | uM |
| | | | ITS | 1:1000 | |
| 3-4 | 7-12 | DMEM + B27 | BMP4 | 50 | ng/ml |
| | | 0.5X | FGF8 | 50 | ng/ml |
| | | | TGFbi IV | 2.5 | uM |
| | | | ITS | 1:200 | |
| 5 | 13+ | DMEM + B27 | KGF | 50 | ng/ml |
| | | 0.5X | ITS | 1:200 | |
| | | | Heparin | 10 | ug/ml |
| | | | Hydro- | 0.5 | ug/ml |
| | | | cortisone | | |
| | | | T3 | 200 | nm |

### Differentiation Factors

The methods and compositions of the present disclosure involve the use of various differentiation factors. Examples of differentiation factors used in the methods and compositions of the present disclosure are described below.

### Activator of RA Receptor

An activator of RA receptor (RAR) may be a molecule capable of activating one or more of RARs, RAR-alpha, RAR-beta, and RAR-gamma. In certain cases, the activator may be a ligand for RA receptor. Examples of ligands of RA receptor include retinoids, such as, retinol, retinal, retinoic acid, all-trans retinoic acid, 9-cis-retinoic acid, etretinate, tazarotene, bexarotene, adapalene, TTNPB, DTAB (3[(4,6-diphenoxy-1,3,5-triazin-2-yl)amino]benzoic acid), or a derivative or analog thereof.

In some embodiments of the methods and compositions described herein, an activator of RA receptor is provided to the cells in a medium such that it is present at a concentration of at least about 0.01 μM, at least about 0.03 μM, at least about 0.1 μM, at least about 0.2 μM, at least about 0.25 μM, at least about 0.3 μM, at least about 1 μM, at least about 1.3 μM, at least about 1.5 μM, at least about 2 μM, at least about 2.3 μM, at least about 2.5 μM, at least about 2.8 μM, at least about 3 μM, at least about 3.5 μM, at least about 4 μM, at least about 4.5 μM, at least about 5 μM, at least about 10 μM, at least about 20 μM, at least about 30 μM, at least about 40 μM or at least about 50 μM.

In certain cases, the activator for RA receptor may be present at different concentrations at different stages of the method for producing TEP cells. In certain cases, the activator for RA receptor may be present at a higher concentration during the generation of DE cells (Stage I) and/or AFE cells (Stage 2) than the concentration in a medium for generating VPE cells (Stage 3) and/or TEP cells (Stage 4).

In certain cases, the activator for RA receptor may be present in the medium used for generating DE cells and in a medium for generating AFE cells at a concentration of about at least about 0.2 μM, at least about 0.25 μM, at least about 0.3 μM, at least about 1 μM, at least about 1.3 μM, at least about 1.5 μM, at least about 2 μM, at least about 2.3 μM, at least about 2.5 μM, at least about 2.8 μM, or at least about 3 μM.

In some case, the activator of RA receptor may be a ligand for RA receptor. In certain cases, a ligand for RA receptor may be all-trans retinoic acid (RA). In certain cases, all trans-retinoic acid may be present at a concentration of 0.25 μM in a cell culture medium used for generating DE cells and in a cell culture medium used for generating AFE cells.

In certain cases, the ligand for RA receptor may be present in the medium used for generating VPE cells and/or TEP cells at a concentration of at least about 0.01 μM, at least about 0.03 μM, at least about 0.1 μM, or at least about 0.15 μM. In certain cases, a ligand for RA receptor may be all-trans retinoic acid (RA). In certain cases, all trans-retinoic acid may be present at a concentration of 0.1 μM in a cell culture medium used for generating VPE cells and in a cell culture medium used for generating TEP cells.

### Fibroblast Growth Factor

In certain embodiments of the methods and compositions described herein, one or more differentiation factors of the fibroblast growth factor family, referred to herein generally as a “fibroblast growth factor” or “FGF”, may be present in the medium used for cell culture. The term “activator of fibroblast growth factor” thus refers to a FGF family protein or a factor that promotes signaling of the FGF pathway. For example, in some embodiments, a fibroblast growth factor can be present in the medium, used for culturing cells, at a concentration of at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example, at a concentration of at least 10 ng/ml, at least 25 ng/ml, at least 50 ng/ml, at least 75 ng/ml, at least 100 ng/ml, at least 200 ng/ml, at least 300 ng/ml, at least 400 ng/ml, at least 500 ng/ml, or at least 1000 ng/ml. In some embodiments, the FGF is present in the cell culture medium at a concentration of 10 ng/ml to 100 ng/ml, such as 20 ng/ml to 100 ng/ml, or 30 ng/ml to 100 ng/ml.

In certain embodiments, the FGF may be FGF2, FGF4, FGF7 (also known as KGF or “keratinocyte growth factor”), FGF8a, FGF8b, FGF9, FGF 10, or a variant thereof.

In certain embodiments, the FGF may be present in a medium used for the generation of VPE cells and/or TEP cells. In certain embodiments, the FGF may be present in a medium used for the generation of VPE cells and/or TEP cells may be FGF8 or FGF8b. In certain embodiments, the FGF may be present in a medium used for the generation of VPE cells and/or TEP cells may be FGF8b, which may be present at a concentration of 50 ng/ml.

In some embodiments of the methods and compositions described herein a fibroblast growth factor may be FGF7/KGF, which may be present in a medium for culturing cells at a concentration of at least about 10 ng/ml, at least about 20 ng/ml, at least about 30 ng/ml, at least about 40 ng/ml, at least about 50 ng/ml, at least about 60 ng/ml, at least about 70 ng/ml, at least about 80 ng/ml, at least about 90 ng/ml, or at least about 100 ng/ml, In some embodiments, the FGF7/KGF is present in the cell culture medium at a concentration of 5 ng/ml to 100 ng/ml, such as 10 ng/ml to 75 ng/ml, or 25 ng/ml to 75 ng/ml.

### Nodal, Activin A, and Activin B

In some embodiments, one or more differentiation factors such as Nodal, and/or Activin A, and/or Activin B or variants thereof or functional analogs thereof can be present in the medium for cell culture at a concentration of at least about 5 ng/ml, at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, such as, about 10-500 ng/ml, 25 ng/ml-250 ng/ml, 50 ng/ml-200 ng/ml.

In some embodiments, one or more differentiation factors such as Nodal, and/or Activin A, and/or Activin B or variants or functional analogs thereof can be present in the medium for generation of DE cells from PS cells (stage 1). In some cases, the medium for generation of DE cells from PS cells (stage 1) may include Act-A at a concentration of 100 ng/ml.

Functional analogs of Activin-A include small molecules, IDE1 (2-[6-carboxy-hexanoyl)-hydrazonomethyl]-benzoic acid), IDE2 (7-(2-cyclopentylidenehydrazino)-7-oxoheptanoic acid described in Borowial M. et al. Cell Stem Cell 4, 348-358, Apr. 3, 2009.

### Wnt Family Members

In certain embodiments of the methods and compositions described herein, one or more differentiation factors of the Wnt family may be present in the medium used for cell culture. For example, in some embodiments, a Wnt family member can be present in the medium, used for culturing cells, at a concentration of at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example, at a concentration of at least 10 ng/ml, at least 25 ng/ml, at least 50 ng/ml, at least 75 ng/ml, at least 100 ng/ml, at least 200 ng/ml, at least 300 ng/ml, at least 400 ng/ml, at least 500 ng/ml, or at least 1000 ng/ml. In some embodiments, the Wnt family member is present in the cell culture medium at a concentration of 5 ng/ml to 100 ng/ml, such as 10 ng/ml to 75 ng/ml, or 15 ng/ml to 50 ng/ml.

In certain cases, the Wnt family member may be present at different concentrations at different stages of the method for producing TEP cells. In certain cases, the Wnt family member may be present at a lower concentration during the generation of DE cells than the concentration in a medium for generating TEP cells. In certain cases, the Wnt family member may be Wnt3a that may be present at a concentration of 25 ng/ml in a cell culture medium used for differentiation of PS cell. In certain cases, the Wnt family member may be Wnt3a that may be present at a concentration of 50 ng/ml in a cell culture medium used for differentiation of AFE cells and for differentiation of VPE cells to produce TEP cells.

In certain cases, the Wnt family member may be an inducer of canonical Wnt signaling. In certain embodiments, the Wnt family member may be Wnt3a or a variant thereof which mediates canonical Wnt signaling. In certain cases, the Wnt family member may be Wnt/beta-catenin pathway agonists, such as, glycogen synthase kinase 3 beta (GSK3b) inhibitors, or casein kinase 1 (CK1) inhibitors. Non-limiting examples of Wnt agonists include DNA encoding β-catenin (e.g., naked DNA encoding β-catenin, plasmid expression vectors encoding β-catenin, viral expression vectors encoding β-catenin), β-catenin polypeptides, one or more Wnt/β-catenin pathway agonists (e.g., Wnt ligands, DSH/DVL-1, -2, -3, LRP6N, WNT3A, WNT5A, and WNT3A, 5A), one or more glycogen synthase kinase 3 (3 (GSK3 (3) inhibitors (e.g., lithium chloride (LiCl), Purvalanol A, olomoucine, alsterpaullone, kenpaullone, benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-dibenzyl-5-oxothiadiazolidine-3-thione (OTDZT), (2′Z,3′E)-6-Bromoindirubin-3′-oxime (BIO), α-4-Dibromoacetophenone (i.e., Tau Protein Kinase I (TPK I) Inhibitor), 2-Chloro-1-(4,5-dibromo-thiophen-2-yl)-ethanone, N-(4-Methoxybenzyl)-N′-(5-nitro-1,3-thiazol-2-yl)urea (AR-A014418), indirubin-5-sulfonamide; indirubin-5-sulfonic acid (2-hydroxyethyl)-amide indirubin-3′-monoxime; 5-iodo-indirubin-3′-monoxime; 5-fluoroindirubin; 5, 5′-dibromoindirubin; 5-nitroindirubin; 5-chloroindirubin; 5-methylindirubin, 5-bromoindirubin, 4-Benzyl-2-methyl-1,2,4-thiadiazolidine-3,5-dione (TDZD-8), 2-thio(3-iodobenzyl)-5-(1-pyridyl)-[1,3,4]-oxadiazole (GSK3 inhibitor II), 2,4-Dibenzyl-5-oxothiadiazolidine-3-thione (OTDZT), (2′Z,3 ‘E)-6-Bromoindirubin-3’-oxime (BIO), a-4-Dibromoacetophenone (i.e., Tau Protein Kinase I (TPK I) Inhibitor), 2-Chloro-1-(4,5-dibromo-thiophen-2-yl)-ethanone, (vi) N-(4-Methoxybenzyl)-N′-(5-nitro-1,3-thiazol-2-yl)urea (AR-A014418), H-KEAPPAPPQSpP-NH2 (L803) and Myr-N-GKEAPPAPPQSpPNH2 (L803-mts)), one or more anti-sense RNA or siRNA that bind specifically to \ddot{υ}8K3β mRNA, one or more casein kinase 1 (CK1) inhibitors (e.g., antisense RNA or siR A that binds specifically to CK1 mPvNA).

Inhibitors of Wnt signaling are used in certain embodiments of the methods described herein. Inhibitors of Wnt signaling include factors that inhibit the Wnt signaling pathway such as Dickkopf (DKK) family proteins, Wnt Inhibitory Factor-1 (WIF-1), and secreted Frizzled-Related Proteins (sFRPs). In some embodiments, small molecule inhibitors of Wnt signaling are used, such as IWP2.

### Activator of BMP Signaling

In certain embodiments of the methods and compositions described herein, one or more differentiation factors, such as, an activator of BMP signaling may be present in the medium used for cell culture. For example, in some embodiments, an activator of BMP signaling can be present in the medium, used for culturing cells, at a concentration of at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml, for example, at a concentration of at least 10 ng/ml, at least 25 ng/ml, at least 50 ng/ml, at least 75 ng/ml, at least 100 ng/ml, at least 200 ng/ml, at least 300 ng/ml, at least 400 ng/ml, at least 500 ng/ml, or at least 1000 ng/ml. In some embodiments, the activator of BMP signaling is present in the cell culture medium at a concentration of 5 ng/ml to 100 ng/ml, such as 10 ng/ml to 75 ng/ml, or 25 ng/ml to 75 ng/ml.

In certain embodiments, the activator of BMP signaling may be BMP1, BMP2, BMP3, BMP4, BMP5, BMP6, BMP7, BMP8a, BMP8b, BMP9/GDF, BMP10, BMP11/GDF11, BMP12/GDF7, BMP13/GDF6, BMP14/GDF5, BMP15/GDF9B, and variants thereof. In certain embodiments, the activator of BMP signaling may be BMP4 or a variant or a functional analog thereof.

### Inhibitors of BMP Signaling

In certain embodiments of the methods and compositions described herein, an inhibitor of BMP signaling may be present in the medium for culturing cells. The inhibitor of BMP signaling may be present at a concentration of at least about 25 μM, at least about 50 μM, at least about 75 μM, at least about 100 μM, at least about 125 μM, at least about 150 μM, at least about 175 μM, at least about 200 μM, at least about 225 μM, at least about 250 μM, at least about 275 μM, at least about 300 μM, at least about 325 μM, at least about 350 μM, at least about 375 μM, at least about 400 μM, at least about 425 μM, at least about 450 μM, at least about 475 μM, or at least about 500 μM, such as 100 μM-150 μM, 150 μM-200 μM, 200 μM-250 μM, 250 μM-300 μM, or 300 μM-350 μM.

In certain embodiments, the inhibitor of BMP signaling may be an antibody or a fragment thereof that binds to BMP1, BMP2, BMP3, BMP4, BMP5, BMP6, BMP7, BMP5, BMP9/GDF2, BMP10, BMP11/GDF11, BMP12/GDF7, BMP13/GDF6, BMP14/GDF5, BMP15, Smad factors, Smad1, Smad5, Smad8, Smad4, Smad6, Smad7, BMP receptors, BMPR-1A (ALK3), BMPR-1B (ALK6), ActR-1A (ALK2, ACVR1), BMPR-2, ActR-2A (ACVR2A), ActR-2B (ACVR2B). In some embodiments, Noggin is used as the inhibitor of BMP signaling. In certain embodiments, the inhibitor of BMP signaling may be a small molecule inhibitor. In certain cases, the inhibitor of BMP signaling may be LDN193189 (“LDN”). In general, the inhibitor of BMP signaling used in the method and compositions disclosed herein does not inhibit Nodal, Activin and/or retinoic acid receptor signaling.

In certain embodiments, LDN may be present in the medium for culturing cells at a concentration of about 250 nM. In some embodiments, LDN may be present at a concentration of at least about 25 nM, at least about 50 nM, at least about 75 nM, at least about 100 nM, at least about 125 nM, at least about 150 nM, at least about 175 nM, at least about 200 nM, at least about 225 nM, at least about 250 nM, at least about 275 nM, at least about 300 nM, at least about 325 nM, at least about 350 nM, at least about 375 nM, at least about 400 nM, at least about 425 nM, at least about 450 nM, at least about 475 nM, or at least about 500 nM, such as 100 nM-150 nM, 150 nM-200 nM, 200 nM-250 nM, 250 nM-300 nM, or 300 nM-350 nM.

### Inhibitors of TGF-β Signaling

In certain embodiments of the methods and compositions described herein, an inhibitor of TGF-β signaling may be present in the medium for culturing cells. The inhibitor of TGF-β signaling may be present at a concentration of at least about 0.01 μM, at least about 0.03 μM, at least about 0.1 μM, at least about 0.2 μM, at least about 0.25 μM, at least about 0.3 μM, at least about 1 μM, at least about 1.3 μM, at least about 1.5 μM, at least about 2 μM, at least about 2.3 μM, at least about 2.5 μM, at least about 2.8 μM, at least about 3 μM, at least about 3.5 μM, at least about 4 μM, at least about 4.5 μM, at least about 5 μM, at least about 10 μM, at least about 20 μM, at least about 30 μM, at least about 40 μM or at least about 50 μM, such as, 0.5 μM-50 μM, 1 μM-25 μM, or 1 μM-10 μM.

In certain embodiments, the inhibitor of TGF-β signaling may be an antibody or a fragment thereof that binds to TGF-βI, TGF-2, TGF-β, TGF-β receptor I and/or II. In certain embodiments, the inhibitor of TGF-β signaling may be a small molecule inhibitor. In certain cases, the inhibitor of TGF-β signaling may be LY364947 (SD208), SM16, SB-505124, ALK5 Inhibitor II, or SB-431542. In some embodiments, the inhibitor of TGF-β signaling is TGF-β RI Kinase Inhibitor IV (Calbiochem). In general, the inhibitor of TGF-β signaling used in the method and compositions disclosed herein does not inhibit Nodal, Activin and/or BMP signaling.

In certain embodiments, TGFbi IV may be present in the medium for culturing cells at a concentration of about 2.5 μM. In some embodiments, TGFbi IV is present in the medium for culturing cells at a concentration of at least about 0.01 μM, at least about 0.03 μM, at least about 0.1 μM, at least about 0.2 μM, at least about 0.25 μM, at least about 0.3 μM, at least about 1 μM, at least about 1.3 μM, at least about 1.5 μM, at least about 2 μM, at least about 2.3 μM, at least about 2.5 μM, at least about 2.8 μM, at least about 3 μM, at least about 3.5 μM, at least about 4 μM, at least about 4.5 μM, at least about 5 μM, at least about 10 μM, at least about 20 μM, at least about 30 μM, at least about 40 μM or at least about 50 μM, such as, 0.5 μM-50 μM, 1 μM-25 μM, or 1 μM-10 μM.

### Inhibitors of Hedgehog Signaling

In certain embodiments of the methods and compositions described herein, an inhibitor of hedgehog signaling may be present in the medium for culturing cells. The inhibitor of hedgehog signaling may be present at a concentration of at least about 0.01 μM, at least about 0.03 μM, at least about 0.1 μM, at least about 0.2 μM, at least about 0.25 μM, at least about 0.3 μM, at least about 1 μM, at least about 1.3 μM, at least about 1.5 μM, at least about 2 μM, at least about 2.3 μM, at least about 2.5 μM, at least about 2.8 μM, at least about 3 μM, at least about 3.5 μM, at least about 4 μM, at least about 4.5 μM, at least about 5 μM, at least about 10 μM, at least about 20 μM, at least about 30 μM, at least about 40 μM or at least about 50 μM, such as, 0.05 μM-5 μM, 0.01 μM-2.5 μM, 0.05 μM-1 μM, or 0.1 μM-1 μM.

In certain embodiments, the inhibitor of hedgehog (Hh) signaling may be an inhibitor of sonic hedgehog (Shh) signaling, desert hedgehog homolog (Dhh) signaling, and/or Indian hedgehog homolog (Ihh) signaling. In certain cases, the inhibitor of hedgehog signaling may be an inhibitor of sonic hedgehog signaling. In certain cases, the inhibitor of hedgehog signaling may be a small molecule. In certain cases, the inhibitor of hedgehog signaling may be a small molecule such as, CU 61414, IPI-926, (Saridegib), IPI-269609, cyclopamine, Vismodegib, or Erismodegib, or derivatives and analogs thereof.

### Additional Factors for Directing Thymic Cell Development

In some embodiments, soluble or other factors can be used in the methods described herein to direct cell fate or development in a culture of cells to thymic cells such as thymic epithelial cells or other cell types within the thymic microenvironment. Certain factors may be present in the medium for culturing cells to support or promote development of a particular subset of thymic cells including, e.g., epithelial cells, mesenchymal cells, pericytes, vascular arterial endothelial cells, vascular venous endothelial cells, lymphatic endothelial cells, red blood cells, immune cells, and/or mesothelial cells (see Table 3). In some embodiments, factors may be present in the medium for culturing cells to support or promote development of a particular subset of epithelial cells, including, e.g., cTEC-hi, cTEC-lo, immature TEC, mTEC-lo, AIRE+ mTEC-hi, keratinocyte-like mTEC, neuroendocrine, myoid, or myelin cells (see Table 5).

Examples of factors that can be added to the culture medium to direct thymic cell development and/or to support particular subsets of thymic cells in the culture include: factors associated with WNT signaling, e.g., WNT5A (ENSG000001142510), WNT6 (ENSG00000115596), ROR1 (ENSG00000185483), ROR2 (ENSG00000169071), RYK (ENSG00000163785), FRZB (ENSG00000162998), RSPO1 (ENSG00000169218), RSPO3 (ENSG00000146374), SFRP2 (ENSG00000145423), and/or SFRP5 (ENSG00000120057); factors associated with BMP signaling, e.g., BMP4 (ENSG00000125378), BMP5 (ENSG00000112175), and/or FST (ENSG00000134363); factors associated with TGF beta signaling, e.g., TGFB1 (ENSG00000105329), TGFBR2 (ENSG00000163513), CXCL12 (ENSG00000107562), and/or CCL21 (ENSG00000137077); factors associated with IGF signaling, e.g., IGF1R (ENSG00000105329); factors associated with FGF signaling pathway, e.g., FGFR2 (ENSG00000066468), and/or FGF7/KGF (ENSG00000140285); factors associated with NOTCH signaling, e.g., NOTCH1 (ENSG00000148400), NOTCH2 (ENSG00000134250), NOTCH3 (ENSG00000074181), HES1 (ENSG00000114315), HES6 (ENSG00000144485), DLL4 (ENSG00000128917), JAG2 (ENSG00000184916), JAG1 (ENSG00000101384), HES2 (ENSG00000069812), HES4 (ENSG00000188290), HEY1 (ENSG00000164683), NRARP (ENSG00000198435), DLK1 (ENSG00000185559), and/or DLK2 (ENSG00000171462); TNF receptors (RANK/TNFRSF11A (ENSG00000141655), CD40 (ENSG00000101017), LTBR (ENSG0000011132), TNFRSF4 (ENSG00000186827), TNFRSF9 (ENSG00000049249), LTB (ENSG00000227507), and/or CD70 (ENSG00000125726)) or their ligands; factors associated with p53 signaling, e.g., PERP (ENSG00000112378), SFN (ENSG00000175793), CTSD (ENSG00000117984), CDKN2A (ENSG00000147889), and/or CDKN2B (ENSG00000147883); and/or Toll-like receptors, e.g., TLR1 (ENSG00000174125), TLR2 (ENSG00000137462), TLR3 (ENSG00000164342), TLR4 (ENSG00000136869), TLR5 (ENSG00000187554), TLR6 (ENSG00000174130), and/or TLR10 (ENSG00000174123).

### Assessing Generation of Cell Populations

In certain cases, the cell populations cultured according to the methods disclosed herein may be monitored to assess changes in the cells imparted by culturing (e.g., during a stage of the culture method disclosed herein) so as to characterize the cell population produced. In certain embodiments, the production of DE cells, AFE cells, VPE cells, TEP cells, and/or TECs, including various sub-populations of TECs, may be assessed by determining the expression of markers characteristic of these cell populations.

In certain cases, the expression of certain markers is determined by detecting the presence or absence of the marker. Alternatively, the expression of certain markers can be determined by measuring the level at which the marker is present in the cells of the cell culture or cell population. In such processes, the measurement of marker expression can be qualitative or quantitative. One method of quantitating the expression of markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest. In certain processes, the expression of marker genes characteristic of the cell population of interest as well as the lack of significant expression of marker genes characteristic of PS cells and other cell types may be determined.

Monitoring of generation of DE cells may be by determining expression of SOX17 gene. As such, the definitive endoderm cells produced by the processes described herein express the SOX17 marker gene, thereby producing the SOX17 gene product. The DE cells produced by the methods described herein also express the Foxa2 gene. Other markers of definitive endoderm include CXCR4, MIXL1, GATA4, HNF3b, GSC, FGF17, VWF, CALCR, FOXQ1, CMKOR1 and CRIP1. Since definitive endoderm cells express the SOX17 marker gene at a level higher than that of the SOX7 marker gene, which is characteristic of primitive and visceral endoderm, in some cases, the expression of both SOX17 and SOX7 may be monitored. In other embodiments, expression of the both the SOX17 marker gene and the OCT4 marker gene, which is characteristic of hESCs, may be monitored. Additionally, because definitive endoderm cells express the SOX17 marker gene at a level higher than that of the AFP, SPARC or Thrombomodulin (TM) marker genes, the expression of these genes can also be monitored.

As such, in some embodiments described herein, the expression of the SOX17 marker and/or the CXCR4 marker in definitive endoderm cells or cell populations is at least about 2-fold higher to at least about 10,000-fold higher than the expression of the SOX17 marker and/or the CXCR4 marker in non-definitive endoderm cells or cell populations, for example pluripotent stem cells. In other embodiments, the expression of the SOX17 marker and/or the CXCR4 marker in definitive endoderm cells or cell populations is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of the SOX17 marker and/or the CXCR4 marker in non-definitive endoderm cells or cell populations, for example pluripotent stem cells.

Markers and methods for identifying DE cells or cell populations are described in U.S. Pat. No. 8,216,836, which is herein incorporated by reference in its entirety.

As noted above, monitoring of generation of AFE cells may be performed by determining expression of SOX2. Monitoring of generation of VPE cells may be performed by determining expression of HOXA3, PAX9, or EYA1. Monitoring of generation of TEP cells may be carried out by determining FOXN1, HOXA3, EYA1, PAX9, and EpCAM.

In certain cases, the monitoring of generation of DE cells, AFE cells, VPE cells, TEP cells, and/or TECs, including various sub-populations of TECs described herein, may be carried out by performing functional analysis of the cells of interest. For example, TEP cells generated by the methods described herein may be functional. Functional TEP cells may generate thymic epithelial cells in vivo or in vitro. In certain cases, functional TEP cells produced by the methods disclosed herein may generate functional TE cells that support T cell development in vivo or in vitro.

Functional analysis of cells can include, for example, identification of marker gene expression. Expression of cell-identifying marker genes can be assessed by detecting mRNA encoding the markers or by detecting all or a part of the marker gene product polypeptide.

In some embodiments, DE cell-identifying markers include: SOX17 and/or FOXA2.

In some embodiments, AFE, cell-identifying markers include: SOX2 and/or FOXA2.

In some embodiments, VPE cell-identifying markers include: PAX9 and/or SIX1.

In some embodiments, TEP cell-identifying markers include: HOXA3, EYA1, EPCAM, K5+, K8+, FOXN1, P63, PAX9 and/or SIX1. For example, TEP cells produced according to the methods described herein can be identified by an increase in the level of expression of FOXN1 by about 1×, about 1.5×, about 2×, about 2.5×, about 3×, or more compared with the level of FOXN1 expression in starting cells, PS cells, DE cells, AFE, cells, and/or VPE cells. TEP cells produced according to the methods described herein can have an increase in the level of expression of KRT5 and/or KRT15 compared with the level of expression in starting cells, PS cells, DE cells, AFE cells, and/or VPE cells. Further, TEP cells produced according to the methods described herein can have a decrease in the level of expression of DLL1 by about 1/1.5×, about 1/2×, about 1/2.5×, about 1/3×compared with the level of DLL1 expression in starting cells, PS cells, DE cells, AFE cells, and/or VPE cells.

In some embodiments, TEC-identifying markers include: EPCAM, K8+ or K5+, FOXN1, P63, PAX9, SIX1 and/or MHC Class II genes.

Cell-identifying markers useful for assessing subsets of thymic epithelial cells are provided below and depicted in Table 1.

In some embodiments, cTEC lineage cells are identified by expression of 135t (PSMB11), CD205 (LY75), CCL25, and/or K8.

In some embodiments, bipotent TEP cells are identified by expression of K5 and/or K8.

In some embodiments, committed mTEC progenitor cells are identified by expression of CLDN3/4 (low), MHC class II genes (negative to low), K5, K15, and/or ASCL1.

In some embodiments, immature mTECs are identified by expression of CLDN3/4, MHC class II genes (low), K5, K15, and/or ASCL1.

In some embodiments, mature mTECs are identified by expression of CLDN3/4, MHC class II genes (high), AIRE, and/or K5.

In some embodiments, post-AIRE mTECs are identified by expression of CLDN3/4, MHC class II genes (low), IVL, and/or K10.

In some embodiments, tuft cells are identified by expression of CLDN3/4, MHC class II genes (low), GNB3, and/or K8.

In some embodiments, neuroendocrine cells are identified by expression of CLDN3/4, MHC class II genes (negative to low), CHGA, SOX2, and/or K8.

In some embodiments, myoid cells are identified by expression of CLDN3/4 (low), MHC class II genes (negative to low), MYOG, and/or K8.

In certain cases, the method does not include monitoring of generation of DE cells, AFE cells, VPE cells, and/or TEP cells.

Enrichment, Isolation and/or Purification of Cell Populations

Cell populations of interest, such as, DE cells, AFE cells, VPE cells, TEP, and/or TECs including subpopulations of thymic epithelial cells produced by any of the methods described herein can be enriched, isolated and/or purified by using an affinity tag that is specific for such cells. Examples of affinity tags specific for a cell or cell population of interest include antibodies, ligands or other binding agents that are specific to a marker molecule, such as a polypeptide, that is present on the cell surface of the cells of interest but which is not substantially present on other cell types that may be found in a cell culture produced by the methods described herein.

Methods for making antibodies and using them for cell isolation are known in the art and such methods can be implemented for use with the antibodies and cells described herein. In one process, an antibody which binds to a marker expressed by cell population of interest is attached to a magnetic bead and then allowed to bind to the cells of interest in a cell culture which has been enzymatically treated to reduce intercellular and substrate adhesion. The cell/antibody/bead complexes are then exposed to a magnetic field which is used to separate bead-bound definitive endoderm cells from unbound cells. Once the cells of interest are physically separated from other cells in culture, the antibody binding is disrupted and the cells are re-plated in appropriate tissue culture medium.

Additional methods for obtaining enriched, isolated, or purified cell populations of interest can also be used. For example, in some embodiments, an antibody for a marker expressed by the cells of interest is incubated in a cell culture containing the cells of interest that has been treated to reduce intercellular and substrate adhesion. The cells are then washed, centrifuged and resuspended. The cell suspension is then incubated with a secondary antibody, such as a FITC-conjugated antibody that is capable of binding to the primary antibody. The cells are then washed, centrifuged and resuspended in buffer. The cell suspension is then analyzed and sorted using a fluorescence activated cell sorter (FACS). Antibody-bound cells are collected separately from cells not bound to the marker specific antibody, thereby resulting in the isolation of cells of interest. If desired, the isolated cell compositions can be further purified by using an alternate affinity-based method or by additional rounds of sorting using the same or different markers that are specific for the cells of interest.

In certain cases, cells of interest, such as, DE cells, AFE cells, VPE cell, TEP cells, TECs, and/or subpopulations of TECs are enriched, isolated and/or purified from other types of cells after the PS cell cultures are induced to differentiate. It will be appreciated that the above-described enrichment, isolation and purification procedures can be used with such cultures at any stage of differentiation.

In addition to the above-described procedures, cells of interest, such as, TEP cells, TECs, and/or subpopulations of TECs may also be isolated by other techniques for cell isolation. Additionally, cells of interest may also be enriched or isolated by methods of serial subculture in growth conditions which promote the selective survival or selective expansion of the cells of interest.

Using the methods described herein, cell populations or cell cultures can be enriched in cells of interest, such as, TEP cells, TECs, and/or subpopulations of TECs, by at least about 2-to about 1000-fold as compared to un-enriched cell populations are produced. For example, in some embodiments, DE cells, and/or AFE cells, and/or VPE cells, and/or TEP cells, and/or TECs and subpopulations thereof can be enriched by at least about 5- to about 500-fold as compared to untreated cell populations or cell cultures. In other embodiments, DE cells, and/or AFE cells, and/or VPE cells, and/or TEP, and/or TECs and subpopulations thereof cells can be enriched from at least about 10- to about 200-fold as compared to untreated cell populations or cell cultures. In still other embodiments, DE cells, and/or AFE cells, and/or VPE cells, and/or TEP cells, and/or TECs and subpopulations thereof can be enriched from at least about 20- to about 100-fold as compared to untreated cell populations or cell cultures. In yet other embodiments, DE cells, and/or AFE cells, and/or VPE cells, and/or TEP, and/or TECs and subpopulations thereof cells can be enriched from at least about 40- to about 80-fold as compared to untreated cell populations or cell cultures. In certain embodiments, DE cells, and/or AFE cells, and/or VPE cells, and/or TEP, and/or TECs and subpopulations thereof cells can be enriched from at least about 2- to about 20-fold as compared to untreated cell populations or cell cultures.

### Genotypic Features of Cell Populations of the Present Disclosure

Single-cell RNA sequencing (scRNA-seq) was used to comprehensively profile the human thymic stroma across multiple stages of life. Analysis of developmental factors expressed by stromal cells led to the identification of mesenchyme and pericytes as potential key regulators of TEC cell fate commitment and differentiation. Sub-clustering of epithelial cells also uncovered previously uncharacterized markers of TEC subsets and revealed the presence of ionocytes, ciliated cells, and myelin-positive cells as novel populations present in the human thymic medulla. Expression of tissue-specific antigens relevant to human autoimmune diseases was mapped to different subsets of TECs, highlighting the potential contribution of specific cell types to the induction of immune tolerance in humans. (See also Park, Jong-Eun, et al. “A cell atlas of human thymic development defines T cell repertoire formation.” Science 367.6480 (2020), incorporated herein by reference in its entirety). These analyses provide crucial information on how the immense diversity of cell types that comprise the thymic microenvironment is established and how this heterogeneity contributes to the induction of immune tolerance in humans.

To unbiasedly define cellular heterogeneity in the human thymic microenvironment, scRNA-seq was used to map the transcriptome of individual stromal cells. Non-lymphoid stromal cells from embryonic, postnatal, and adult tissue were analyzed to investigate cell fate specification and TEC maturation processes at different time points during development. These studies identified novel candidate pathways that regulate TEC fate commitment and uncovered previously uncharacterized TEC markers, revealing the presence of distinct mTEC subsets (AIRE+, corneocyte-like, CCL21+, and tuft). Additionally, ciliated cells, myelin+ neuronal cells, and CFTR+ ionocytes were identified as new subsets of epithelial cells present in the human thymic medulla that do not have known murine counterparts. Expression of disease-relevant genes in the epithelial compartment was analyzed in an effort to better understand how immune tolerance is established in the human thymus. The results of the scRNA-seq analysis described herein give rise to novel subsets of marker genes and genotypic features of cell populations produced by the methods disclosed herein including TEP cells, TECs, and subpopulations of TECs.

When derived from an isolated PS cell, or an established line of PS cells, the cell populations of this disclosure can be characterized as being the progeny of the originating cell or cell line. Accordingly, the cell populations will have the same genome as the cells from which they are derived. This means that over and above any karyotype changes, the chromosomal DNA will be over 98% (e.g., at least 98.5%, 98.8%, 99%, 99.3%, 99.5%, 99.9%, or more) identical between the PS cells and the cell populations generated therefrom. Cell populations of the present disclosure that have been treated by recombinant methods to introduce a transgene or knock out an endogenous gene are still considered to have the same genome as the line from which they are derived, since all non-manipulated genetic elements are preserved. Cell populations of the present disclosure and PS cells can be identified as having the same genome by standard genetic techniques. Possession of the same genome can also be inferred if the cell populations are obtained from the undifferentiated line through the course of normal mitotic division.

In certain industrial applications, this characteristic is a valuable feature of the cell populations of the present disclosure. In particular, the availability of the originating PS cells provides a further supply of genetically matched differentiated cell populations, since the PS cells can be caused to proliferate and differentiated into more cell populations of the present disclosure as required. Furthermore, the PS cells can be differentiated into other therapeutically important lineages.

The techniques described in this application allow for the production of large cell populations that share the same genome, by expanding the cells before or after differentiation.

Populations of 10\hat{ }8, 10\hat{ }10, or 10\hat{ }12 cells are theoretically possible. Such large populations are usually divided into separate containers suitable for further culture, drug screening, or therapeutic administration.

Certain embodiments of the disclosure include originating cells (such as an undifferentiated PS cell line, or an intermediate population, e.g., DE cells, AFE cells, VPE cells, TEP cells) in combination with one or more populations of differentiated cells bearing characteristics of DE cells, AFE cells, VPE cells, TEP cells, or TECs, or subpopulations thereof. The populations may either be in the same container, in separate containers in the same facility, or in two different locations. The undifferentiated and differentiated cells may be present simultaneously or at a different time, such as when a culture of undifferentiated cells is caused to differentiate into TEP cells, as described herein.

### Compositions and Systems Comprising Cell Populations

### Cell Compositions

The present disclosure provides compositions comprising a differentiated population of cells. The compositions may comprise differentiated TECs, wherein the differentiated TECs are derived from PS cells and comprise one or more of a subpopulation of TECs, including cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin positive cells, and/or myoid cells.

Also provided are compositions comprising reaggregated thymic epithelial progenitor (TEP) cells in combination with one or more cell type selected from lymphatic endothelium cells, vascular endothelium cells, immune cells, mesenchymal cells, pericytes, red blood cells, or combinations thereof.

Also provided are compositions comprising reaggregated thymic epithelial cells (TECs) in combination with one or more cell type selected from lymphatic endothelium cells, vascular endothelium cells, immune cells, mesenchymal cells, pericytes, red blood cells, or combinations thereof.

The compositions provided herein may be produced according to the methods described herein.

The compositions comprising TECs provided herein may further comprise subpopulations of TECs including one or more of cTEC lineage cells, bipotent TEP cells, committed mTEC progenitor cells, immature mTECs, mature mTECs, post-AIRE mTECs, tuft cells, neuroendocrine cells, and/or myoid cells. Thus, compositions comprising thymic epithelial cells recapitulating the complexity of the endogenous thymic microenvironment are provided. In some embodiments, the compositions of TEP cells and/or TECs include various subsets of TECs, including for example the subpopulations described in Table 1. In some embodiments, the compositions comprise populations of thymic epithelial cells having diverse subpopulations of epithelial cells similar to those found in human fetal, post-natal, or adult thymic tissue. For example, the compositions comprising TEP cells and/or TECs described herein comprise subpopulations representing one or more of the subpopulations of thymic epithelial cells shown in Table 1.

The compositions may further comprise reaggregated TEP cells and/or TECs.

Cell compositions produced by the methods described herein include cell cultures that contain isolated TEP cells and cell populations enriched in isolated TEP cells, isolated TECs and cell populations enriched in isolated TECs. In certain cases, the cell composition including isolated TEP cells and/or TECs may further include one or more of an activator of RA receptor, an activator of BMP signaling, a Wnt family member, a fibroblast growth factor, and an inhibitor of hedgehog signaling.

In general, the TEP cells of the present disclosure present in the systems, cell populations, and compositions described herein are functional. In certain embodiments, the TEP cells are functional and further differentiate into TE cells under appropriate conditions, in vivo or in vitro. The functional activity of the TEP cells may be assessed by any of the methods described herein or any of the art accepted methods, such as, those described in Inami Y et al, Immunology and Cell Biology (2011) 89, 314-321; Lai L. and Jin J., Stem Cells. 2009 December; 27(12):3012-20; Lai L. et al, Blood. 2011 Sep. 22; 118(12):3410-8. For example, the functional TEP cells may further mature upon transplantation into functional TE cells that support T cell development.

TECs produced according to the methods described herein may contain subpopulations of epithelial cells, including one or more subpopulation as described in Table 1, and thereby recapitulate the biological complexity of endogenous thymic tissue.

In certain embodiments, the TEP cells of the present disclosure present in the systems, cell populations, and compositions described herein express one or more of markers of TEP cells, which markers are present in TEP cells present in thymus or thymic tissue, such as, adult human thymus or fetal human thymus. For example, TEP cells produced by the methods described herein may express the TEP markers at a level similar to the level expressed by TEP cells in adult or fetal thymus. In certain cases, the TEP cells of the present disclosure express one or more of FOXN1, HOXA3, EYA1, GCM2, and EpCAM. In certain cases, the TEP cells produced by the methods provided herein express FOXN1. In certain cases, the TEP cells produced by the methods provided herein express HOXA3. In certain cases, the TEP cells produced by the methods provided herein express FOXN1 and HOXA3. In certain cases, the TEP cells produced by the methods provided herein express FOXN1, HOXA3, and EpCAM. In certain cases, the TEP cells produced by the methods provided herein express FOXN1, HOXA3, and EYA1. In certain cases, the TEP cells produced by the methods provided herein express FOXN1, HOXA3, PAX1, EpCAM, and EYA1. In certain cases, the TEP cells provided herein do not express significant levels of marker genes characteristic of mature TECs such as HLA-DRA (MHC class II molecule) and AIRE. Detection of expression of one or more of FOXN1, HOXA3, EYA1, GCM2, and EpCAM can be accomplished according to the methods known in the art, such as those discussed herein.

As such, the TEP cells of the present disclosure express one or more of the markers provided herein and are functional.

As noted herein the TEP cells of the present disclosure may be mammalian, e.g., primate TEP cells, such as, human TEP cells.

In certain embodiments, the TECs and/or subpopulations thereof of the present disclosure present in the systems, cell populations, and compositions described herein express one or more markers of TECs, which markers are present in TECs present in thymus or thymic tissue, such as, adult human thymus or fetal human thymus. For example, TECs produced by the methods described herein may express the TEC markers at a level similar to the level expressed by TEC cells in adult or fetal thymus. In certain cases, the TECs of the present disclosure express one or more of β5t (PSMB11), CD205 (LY75), CCL25, K8, K5, K10, K15, CLDN3/4, MHCII, ASCL1, AIRE, IVL, GNB3, CHGA, and/or SOX2. In certain cases, the TECs produced by the methods provided herein express β5t (PSMB11). In certain cases, the TECs produced by the methods provided herein express CD205 (LY75). In certain cases, the TECs produced by the methods provided herein express CCL25. In certain cases, the TECs produced by the methods provided herein express K8. In certain cases, the TECs produced by the methods provided herein express K5. In certain cases, the TECs produced by the methods provided herein express K10. In certain cases, the TECs produced by the methods provided herein express K15. In certain cases, the TECs produced by the methods provided herein express CLDN3/4. In certain cases, the TECs produced by the methods provided herein express MHC class II genes. In certain cases, the TECs produced by the methods provided herein express ASCL1. In certain cases, the TECs produced by the methods provided herein express AIRE. In certain cases, the TECs produced by the methods provided herein express IVL. In certain cases, the TECs produced by the methods provided herein express GNB3. In certain cases, the TECs produced by the methods provided herein express CHGA. In certain cases, the TECs produced by the methods provided herein express SOX2.

As such, the TECs of the present disclosure express one or more of the markers provided herein, i.e., in Table 1 and/or Table 2 and are functional.

As noted herein the TECs of the present disclosure may be mammalian, e.g., primate TEP cells, such as, human TECs.

Cell compositions produced by the described methods include cell cultures that include isolated VPE cells and cell populations enriched in VPE cells. In certain cases, the cell composition containing VPE cell may include one or more of an activator of RA receptor, an activator of BMP signaling, a Wnt family member, a fibroblast growth factor, and an inhibitor of hedgehog signaling. In certain cases, the cell composition of VPE cells may include one or more of an activator of RA receptor, an activator of BMP signaling, an inhibitor of TGF-β signaling, a Wnt family member, a fibroblast growth factor, and an inhibitor of hedgehog signaling. In general, the VPE cells present in the cell populations are capable of differentiating into TEP cells, when cultured according to the methods disclosed herein.

Cell compositions produced by the described methods include cell cultures that include AFE, cells and cell populations enriched in AFE cells. In certain cases, the cell composition comprising AFE cell may include one or more of an activator of RA receptor, an activator of BMP signaling, an inhibitor of TGF-β signaling, a Wnt family member, a fibroblast growth factor, and an inhibitor of hedgehog signaling. In general, the AFE cells present in the cell populations are capable of differentiating into VPE cells, and TEP cells, when cultured according to the methods disclosed herein.

Cell compositions produced by the described methods include cell cultures that include DE cells and cell populations enriched in DE cells. In certain cases, the cell composition comprising DE cell may include one or more of an activator of RA receptor, an activator of BMP signaling, and an inhibitor of TGF-β signaling. In certain cases, the cell composition comprising DE cell may include one or more of an activator of RA receptor, Nodal, Act-A, Act-B. In general, the DE cells present in the cell populations are capable of differentiating into AFE cells, VPE cells, and TEP cells, when cultured according to the methods disclosed herein.

Cell compositions produced by the described methods include cell cultures that include isolated TEP cells and cell populations enriched in TEP cells. In certain cases, the cell composition containing TEP cells may include one or more of an activator of RA receptor, an activator of BMP signaling, a Wnt family member, a fibroblast growth factor, and an inhibitor of hedgehog signaling.

Cell compositions produced by the described methods include cell cultures that include isolated TECs and cell populations enriched in TECs and subpopulations thereof.

In some embodiments, cell compositions which include cells of the present disclosure (e.g., TECs, TEP cells, or VPE cells, or AFE cells, or DE cells), wherein at least about 50%-80% of the cells in culture are the cells of interest, can be produced. The differentiation methods described herein can result in about 5%, about 10%, about 15%, about 20%>, about 25%, about 30%>, about 35%, about 40%, about 45%, about 50%, about 55%, about 60%, about 65%, about 70%, about 75%), about 80%>, about 85%, about 90%>, about 95%, or greater than about 95% conversion of pluripotent cells to cells of interest.

In embodiments, in which isolation of cells of interest is employed, for example, by using an affinity reagent that binds to the cells of interest, a substantially pure cell population of interest can be recovered.

Some embodiments described herein relate to cell compositions comprising from at least about 5% cells of interest to at least about 95% cells of interest. In some embodiments, the cell cultures or cell populations comprise mammalian cells. In preferred embodiments, the cell cultures or cell populations comprise human cells. For example, certain specific embodiments relate to cell compositions comprising human cells, wherein from at least about 5% to at least about 95% of the human cells are TECs. Other embodiments relate to cell compositions comprising human cells, wherein at least about 5%, at least about 10%, at least about 15%, at least about 20%, at least about 25%, at least about 30%, at least about 35%, at least about 40%, at least about 45%, at least about 50%, at least about 55%, at least about 60%, at least about 65% o, at least about 70%, at least about 75%, at least about 80%, at least about 85%, at least about 90% or greater than 90% of the human cells are TECs.

Cell compositions produced by the above-described methods and compositions thereof may be assessed by using the markers and methods described herein as well as those known in the art.

Cell compositions produced by the above-described methods and compositions thereof may be enriched, isolated or purified using methods described herein as well as those known in the art.

Cell compositions provided herein may be pharmaceutical compositions that include a pharmaceutically acceptable carriers. Examples of pharmaceutically acceptable carriers include saline, buffers, diluents, fillers, salts, stabilizers, solubilizers, cell culture medium, and other materials which are well known in the art. In some embodiments, the formulations are free of detectable DMSO (dimethyl sulfoxide).

For general principles in medicinal formulation of cell compositions, the reader is referred to Cell Therapy: Stem Cell Transplantation, Gene Therapy, and Cellular Immunotherapy, by G. Morstyn & W. Sheridan eds, Cambridge University Press, 1996; and Cell Transplantation for Neurological Disorders, T. B. Freeman et al. eds., Humana Press 1998. The cells may be packaged in a device or container suitable for distribution or clinical use, optionally accompanied by information relating to the storage of the cells or their use as a medicament to treat clinical conditions, or for any other worthwhile purpose.

Also provided herein is a first in vitro cell population including primate (e.g., human) pluripotent stem cells and a second in vitro cell population comprising progeny of a portion of the first in vitro cell population, wherein the progeny are TEP cells and/or TECs as described herein.

Accordingly, the TEP cells and/or TECs in the second in vitro cell population may be functional and express the markers provided herein.

The first and second in vitro cell populations may exist at the same time or at different times. The first and second in vitro cell populations may be present in the same container or in different containers.

In certain cases, the first in vitro cell population may be pPS cells, DE cells, AFE cells or VPE cell and the second in vitro cell population may be TEP cells and/or TECs, where the TEP cells and or TECs are progeny of the pPS cells, DE cells, AFE cells or VPE cells.

In certain cases, the first in vitro cell population may be DE cells and the second in vitro cell population may be AFE cells, where the AFE cells are progeny of the DE cells.

The first in vitro cell population may be DE cells and the second in vitro cell population may be VPE cells, where the VPE cells are progeny of the DE cells.

The first in vitro cell population may be AFE cells and the second in vitro cell population may be VPE cells, where the VPE cells are progeny of the AFE cells.

Also provided herein is a first, second, and third in vitro cell populations, where the first cell population may be AFE cells, the second cell population may be VPE cells and the third cell population may be TEP cells, where the VPE cells are progeny of AFE cells and TEP cells are progeny of VPE cells.

Also provided herein is a first, second, third, and fourth in vitro cell populations, where the first cell population may be DE cells, the second cell population may be AFE cells, the third cell population may be VPE cells and the fourth cell population may be TEP cells, where the AFE cells are the progeny of DE cells, the VPE cells are progeny of AFE cells and TEP cells are progeny of VPE cells.

Also provided herein is a first, second, third, fourth, and fifth in vitro cell populations, where the first cell population may be pPS cells, the second cell population may be DE cells, the third cell population may be AFE cells, the fourth cell population may be VPE cells and the fifth cell population may be TEP cells, where the DE cells are progeny of the pPS cells, the AFE cells are the progeny of DE cells, the VPE cells are progeny of AFE cells and TEP cells are progeny of VPE cells.

Some embodiments of the cell compositions described include one or more populations of cells including populations of DE cells, of AFE cells, of VPE cells, of TEP cells, and/or of TECs, or subpopulations thereof.

### Systems

Also provided herein is a system for efficient production of primate TEP cells and/or TECs and subpopulations thereof for use in research or the preparation of pharmaceutical compositions for treatment of a subject in need of treatment with TEP cells.

The systems of the present disclosure include a set or combination of cells that exist at any time during manufacture, distribution, or use. The cell sets comprise any combination of two or more cell populations described in this disclosure, exemplified but not limited to a type of differentiated pPS-derived cell (such as, TECs and/or subpopulations thereof, TEP cells, VPE cells, AFE cells, DE cells), in combination with undifferentiated pPS cells or other differentiated cell types, sometimes sharing the same genome. Each cell type in the set may be packaged together, or in separate containers in the same facility, or at different locations, at the same or different times, under control of the same entity or different entities sharing a business relationship.

In certain embodiments, a differentiated cell population as part of a system for generating TEP cells and/or TECs and subpopulations thereof is provided. The TEP cells and/or TECs and subpopulations thereof of the system have functional and phenotypic characteristics (e.g., expression of TEP cell markers) as provided herein and are the progeny of primate pluripotent stem (pPS) cells. In other words, the TEP cells and/or TECs and subpopulations thereof of the system are produced by differentiation of pPS cells.

In exemplary embodiments, the system of components for generating TEP cells and/or TECs and subpopulations thereof may include a line of undifferentiated human PS cells and a cell population of TEP cells and/or TECs and subpopulations thereof differentiated therefrom, wherein the TEP cells and/or TECs and subpopulations thereof express one or more of the TEP cell markers, such as those provided herein.

The system of components for generating TEP cells and/or TECs and subpopulations thereof may include human AFE cells and a cell population of TEP cells and/or TECs and subpopulations thereof differentiated therefrom, wherein the TEP cells and/or TECs and subpopulations thereof express one or more of the TEP cell markers, such as those provided herein.

The system of components for generating TEP cells and/or TECs and subpopulations thereof may include human VPE cells and a cell population of TEP cells and/or TECs and subpopulations thereof differentiated therefrom, wherein the TEP cells and/or TECs and subpopulations thereof express one or more of the TEP cell markers, such as those provided herein.

The system of components for generating TEP cells and/or TECs and subpopulations thereof may include human PS cells and a cell population of DE cells differentiated therefrom, wherein the DE cells express one or more of the DE cell markers, such as those provided herein.

The system of components for generating TEP cells and/or TECs and subpopulations thereof may include human PS cells and a cell population of AFE, cells differentiated therefrom, wherein the AFE cells express one or more of the AFE cell markers, such as those provided herein.

The system of components for generating TEP cells and/or TECs and subpopulations thereof may include human PS cells and a cell population of VPE cells differentiated therefrom, wherein the VPE cells express one or more of the VPE cell markers, such as those provided herein.

The system of components for generating TEP cells and/or TECs and subpopulations thereof may include human PS cells, a cell population of DE cells differentiated from the PS cells, a cell population of AFE cells differentiated from the DE cells, cell population of VPE cells differentiated from the AFE cells, and a cell population of TEP cells and/or TECs and subpopulations thereof differentiated from the AFE cells, wherein the cell populations express one or more markers typical for the particular cell, such as, those described herein.

The cell population of TEP cells and/or TECs and subpopulations thereof of the system and compositions described herein may include at least 10%-95% or more TEP cells and/or TECs and subpopulations thereof (e.g., 15%-90%, 20%-80%, 50%-70%, 10%, 20%, 30%, 40%, 50%, 60%, 70%, 80%, 90%, 95%, or 99%).

The cell population of VPE cells of the system and compositions described herein may include at least 10%-95% or more VPE cells (e.g., 15%-90%, 20%-80%, 50%-70%, 10%, 20%, 30%, 40%, 50%, 60%, 70%, 80%, 90%, 95%, or 99%).

The cell population of AFE cells of the system and compositions described herein may include at least 10%-95% or more AFE cells (e.g., 15%-90%, 20%-80%, 50%-70%, 10%, 20%, 30%, 40%, 50%, 60%, 70%, 80%, 90%, 95%, or 99%).

The cell population of DE cells of the system and compositions described herein may include at least 10%-95% or more DE cells (e.g., 15%-90%, 20%-80%, 50%-70%, 10%, 20%, 30%, 40%, 50%, 60%, 70%, 80%, 90%, 95%, or 99%).

### Uses of Cell Populations

### Cell Populations for Screening

The cells of the present disclosure can be used to screen for agents (such as, small molecules, peptides, polynucleotides) or environmental conditions (such as, culture conditions or manipulation) that affect the characteristics of PS cells, DE cells, AFE cells, VPE cells, TEP cells, and/or TECs and subpopulations thereof according to the present description.

In one example, PS cells, DE cells, AFE cells, and/or VPE cells (undifferentiated or initiated into the differentiation paradigm) are used to screen factors that promote maturation into TEP cells, or promote proliferation and maintenance of TEP cells and/or TECs and subpopulations thereof in long-term culture. For example, candidate differentiation factors or growth factors are tested by adding them to cells in different wells, and then determining any phenotypic change that results, according to desirable criteria for further culture and use of the cells. This can lead to improved derivation and culture methods for generating DE cells, AFE cells, VPE cells, TEP cells, and/or TECs.

Other screening methods of the present disclosure relate to the testing of pharmaceutical compounds for a potential adverse effect on TEP cells and/or TECs. This type of screening is appropriate not only when the compound is designed to have a pharmacological effect on TEP cells and/or TECs and subpopulations thereof themselves, but also to test for TEP cells/TE cells-related side-effects of compounds designed for a primary pharmacological effect elsewhere.

Other screening methods relate to the use of TEP cells and/or TECs and subpopulations thereof to measure the effect of small molecule drugs that have the potential to affect immune system. To this end, the cells can be combined with test compounds in vitro, and the effect of the compound on TEP cells and/or TECs and subpopulations thereof is determined.

General principles of drug screening are described in U.S. Pat. No. 5,030,015, and in the textbook In vitro Methods in Pharmaceutical Research, Academic Press 1997. Assessment of the activity of candidate pharmaceutical compounds generally involves combining the differentiated cells of this invention with the candidate compound, either alone or in combination with other drugs. The investigator determines any change in the morphology, marker, or functional activity of the cells that is attributable to the compound (compared with untreated cells or cells treated with a negative control compound), and then correlates the effect of the compound with the observed change.

TEP Cells and/or TECs and Subpopulations Thereof in Clinical Therapy

Cell populations comprising TEP cells, such as, cell populations enriched in TEP cells, as well as, purified TEP cells and/or TECs and subpopulations thereof produced by the methods described herein may be used in a number of clinical applications.

In certain embodiments, the TEP cells and/or TECs and subpopulations thereof produced using the methods provided herein may be used for generating functional thymic epithelium (TE) in a subject in need thereof.

A subject in need of TEP cells and/or TECs and subpopulations thereof may be a subject having a genetic and/or developmental defect that results in reduced or undetectable thymus functions. In certain cases, the subject may have DiGeorge syndrome or complete DiGeorge syndrome. Complete DiGeorge syndrome is a fatal condition in which infants have no detectable thymus function. The TEP cells and/or TECs and subpopulations thereof of the present disclosure may be used for treatment of infants with complete DiGeorge syndrome. For example, infants with complete DiGeorge syndrome may be treated using the instant TEP cells and/or TECs and subpopulations thereof by following the transplantation procedure described by Markert M. L. et al., Blood. 2003 Aug. 1; 102(3): 1121-30. Epub 2003 Apr. 17.

In certain embodiments, the TEP cells and/or TECs and subpopulations thereof produced using the methods provided herein may be used in thymus regeneration therapy.

The TEP cells and/or TECs and subpopulations thereof may be transplanted into a subject in need of TE cells. In certain cases, the TEP cells and/or TECs and subpopulations thereof may be transplanted into a target site in a subject that provides appropriate differentiation conditions for the TEP cells and/or TECs and subpopulations thereof to differentiate into TE cells. Cells may be transplanted by any of a number of standard methods in the art for delivering cells to tissue, e.g., injecting them as a suspension in a suitable buffer (saline, PBS, DMEM, Iscove's medium, etc. or a pharmaceutically acceptable carrier), providing them on a solid support, e.g. a bead, a filter such as a mesh filter, a membrane, etc. In certain cases, the TEP cells and/or TECs and subpopulations thereof may be transplanted into the thymus of a subject. In certain cases, the TEP cells and/or TECs may be transplanted under the kidney capsule of a subject.

In certain cases, a subject in need of TEP cell and/or TEC transplantation may be a subject that needs an increase in enhancement or restoration of thymic function. In certain cases, the subject may be a subject whose thymus has undergone profound degeneration due to aging. In certain cases, the subject may be a subject whose thymus has undergone profound degeneration due to exposure to radiation. In certain cases, the subject may be a subject whose thymus has undergone profound degeneration due to chemotherapy.

In certain cases, thymic tissue or thymic epithelial cell populations produced according to the methods described herein can be used to develop T lymphocytes for use in therapeutic applications. In some embodiments, thymic tissue or thymic epithelial cell populations produced according to the methods described herein can be used to support the development of a diverse, self-tolerant peripheral T cell pool.

In certain cases, thymic tissue or thymic epithelial cell populations produced according to the methods described herein can be used to promote and/or manipulate immune tolerance in the context of various therapeutic applications such as therapies for autoimmune conditions, including, e.g., type 1 diabetes. In some embodiments, the thymic tissue or thymic epithelial cell populations produced according to the methods described herein can be used to provide for methods of promoting, reducing, or manipulating immune tolerance in a human subject. For example, AIRE+ mTECs as described herein may participate in induction of tolerance by providing antigens that can be presented by antigen-presenting cells like dendritic cells. For example, myoid cells may participate in the induction of immune tolerance to muscle antigens. For instance, many thymoma patients who typically lack myoid cells develop myasthenia gravis (MG), an autoimmune disease of the neuromuscular junction characterized by autoantibodies to the acetylcholine receptor (AChR) or other muscle antigens like titin (TTN). APS-I patients, on the other hand, typically do not have detectable autoantibodies against either AChR or TTN, suggesting that the expression of these antigens is not entirely AIRE dependent. Importantly, expression of AChR and TTN was shown to be much higher in myoid cells compared to AIRE+ mTECs, supporting the idea that myoid cells are the main source of muscle antigens in the human thymic medulla. Thus, the methods provided herein can be used to manipulate, i.e., tune up or down, autoimmune tolerance by titrating relative presence of subpopulations of thymic epithelial cells present in compositions according to the present description. Alternatively, the methods provided herein establish a roadmap for directing development of thymic epithelial cells based on presence of certain factors and combinations of factors present in thymic tissue, allowing for new interventions for correcting diseased or deficient thymic tissues in vivo. These findings thus provide methods for regulation of disease-relevant tissue-specific antigens in the human thymus.

In some embodiments, the thymic tissue or thymic epithelial cell populations produced according to the methods described herein can be used to repair or replenish deficient thymic tissue in a subject. In some embodiments, the compositions and methods described provide therapeutic compositions and methods which may help to establish and maintain healthy thymic tissue through the course of disease or aging.

TEPs generated from patient-specific induced pluripotent stem (iPS) cell lines may also be used as a tool to model human disease.

In certain cases, the TEPs generated by the method described herein may be genetically modified to express a protein of interest.

The present invention is further illustrated by the following non-limiting examples.

### EXAMPLES

Methods of producing TEPs and TECs are described, for example, in International Patent Application Publication Number WO2014134213, incorporated herein by reference in its entirety.

### Methods

### Thymic Tissue Acquisition

Human fetal thymic tissues were obtained from 19-23 gestational week specimens under the guidelines of the Committee on Human Research (UCSF IRB)—approved protocols from the Department of Obstetrics, Gynecology and Reproductive Science, San Francisco General Hospital. Pediatric tissues were obtained from patients undergoing corrective cardiothoracic surgery in accordance with protocols approved by the UCSF Human Research Protection Program Institutional Review Board. Human adult thymic tissues were acquired from research consented deceased organ donors at the time of organ acquisition for clinical transplantation through an IRB-approved research protocol with Donor Network West, the organ procurement organization for Northern California. All donors were free of chronic disease and cancer and were negative for Hepatitis B/C and HIV.

### Tissue Preparation

Thymic tissues placed in RPMI (ThermoFisher) containing 100 μg/ml DNase I (Roche) were cut into small pieces using scissors. Tissue pieces were transferred into a gentleMACS C tube (Miltenyi) containing 10 ml of RPMI with DNAse. The gentleMACS Program m_spleen_02 was run three times. Thymic fragments were separated from the thymocytes-rich supernatant by centrifugation. Remaining fragments were transferred back to C tube with fresh RPMI with DNAse before running program m_spleen_01. The supernatant was removed and replaced with 10 ml of digestion medium containing 100 μg/ml DNase I and 100 μg/ml Liberase TM (Sigma-Aldrich) in RPMI. Tubes were moved to a 37° C. water bath and fragments were triturated every 5 minutes to mechanically aid digestion. At 30 min, tubes were spun briefly to pellet undigested fragments and the supernatant was discarded. Fresh digestion medium or accumax (STEMCELL Technologies) was added to remaining fragments and the digestion was repeated for another 15-30 minutes until most pieces were digested. Supernatant from this second round of digestion was transferred to a tube containing cold MACS buffer (0.5% BSA, 2 mM EDTA in PBS) to stop the enzymatic digestion. If necessary, a third round of enzymatic digestion was performed on remaining fragments using accumax for an additional 5-10 min. Cells were pooled with the supernatant from the previous round of digestion and were passed through a 40 μm filter (Falcon). Some samples were treated with 2 ml of ACK lysing buffer (Lonza) for 5 min prior to stromal enrichment.

### Enrichment of Stromal Cells

For fetal and postnatal tissues, single cells from digested tissue were resuspended in MACS buffer containing 10 μM of the ROCK inhibitor Y-27632 (Tocris). Human CD45 MicroBeads (Miltenyi) were used to deplete immune cells according to the manufacturer's instructions with the following modification: 5 μL of CD45 MicroBeads per 10\hat{ }7 total cells were added instead of 20 μL. LD columns were used for depletion and the CD45 negative fraction was collected in MACS buffer. Stromal cells from adult thymus were enriched using Fluorescence activated cell sorting (FACS). Blocking was done with human Fc Receptor Binding Inhibitor Monoclonal Antibody (eBioscience) followed by staining for 20 min using human-specific antibodies against EPCAM (Clone 1B7, eBioscience) and CD45 (Clone HI30, Biolegend). After staining, cells were washed and resuspended in FACS buffer containing DAPI. Cells were sorted on BD FACS Aria II. Pre-gating was first done for live cells based the DAPI stain.

### Single-Cell RNA-Seq and Computational Analysis

Single cells were captured using the 10×Chromium microfluidics system (10×Genomics). The cells were encapsulated and barcoded cDNA libraries were prepared using the single-cell 3′ mRNA kit (v2 or v3; 10×Genomics). Single-cell libraries were sequenced using a NovaSeq 6000. The Cell Ranger software pipeline (10×Genomics, version 2.0.0, 2.1.1, or 3.0.2) was used to demultiplex cellular barcodes, map reads to the human genome (GRCh38), and transcriptome using the STAR aligner, and produce a matrix of gene counts versus cells. Single-cell data analysis was performed using SCANPY (Wolf, F. A., Angerer, P. & Theis, F. J. SCANPY: large-scale single-cell gene expression data analysis. Genome Biol. 19, 15-5 (2018)). Cell by gene count matrices of all samples were concatenated to a single matrix. Cells with 200-5000 detected genes and expressing <10% mitochondrial genes as well as genes expressed in >3 cells were retained (68008 cells total with an average of 4769 counts per cell). Counts were log transformed and total counts per cell were normalized. The dataset was filtered for highly variable genes (minimum mean=0.0125, maximum mean=3, and dispersion, 0.5 per gene) and variation caused by mitochondrial gene expression and cell cycle-dependent changes in gene expression were regressed out. BBKNN (Polański, K. et al. BBKNN: Fast Batch Alignment of Single Cell Transcriptomes. Bioinformatics 36, 411. (2019)) was applied to correct donor-specific effects. K-nearest neighbor graphs were constructed (n_neighbors=15) and clustering was performed using the Leiden algorithm with a resolution of 0.5. Clustering results were visualized using Uniform Manifold Approximation and Projection (UMAP).

### Pseudotime Analysis and Gene Scores

Pseudotime analysis was performed using RNA Velocity (La Manno, G. et al. RNA velocity of single cells. Nature 560, 494-498 (2018)). Spliced and unspliced expression matrices were generated using the standard velocyto pipeline. The following steps were performed using the scVelo package (Bergen, V., Lange, M., Peidli, S., Wolf, F. A. & Theis, F. J. Generalizing RNA velocity to transient cell states through dynamical modeling. bioRxiv 820936 (2019)). The matrices were size-normalized to the median of total mRNA molecules across all cells. Genes were selected based on a threshold of a minimum of 20 expressed counts for both spliced and unspliced mRNA. The top 2000 highly variable genes were kept for further downstream analysis. Nearest neighbor graphs were calculated with 30 neighbors based on the normalized gene expression matrices from the original analysis. Velocity estimations were calculated using the standard scVelo pipeline and the resulting velocity graphs were projected onto the UMAPs previously generated using SCANPY. The TSA and APS-1 scores were calculated using the scanpy.tl.score_genes function. This function calculates the average expression of a set of genes subtracted with the average expression of a randomly selected reference set of genes (Satija, R., Farrell, J. A., Gennert, D., Schier, A. F. & Regev, A. Spatial reconstruction of single-cell gene expression data. Nature Biotechnology 33, 495-502 (2015)). TSA genes were identified using data from the GNF Mouse GeneAtlas as reported by Sansom, S. N. et al. Genome Res. 24, 1918-1931 (2014). APS-1 genes were selected based on their association with auto-antibodies in APS-1 patients (Constantine, G. M. & Lionakis, M. S. Lessons from primary immunodeficiencies: Autoimmune regulator and autoimmune polyendocrinopathy-candidiasis-ectodermal dystrophy. Immunol. Rev. 287, 103-120 (2019)). The average expression level of scored genes was visualized using UMAP.

### Immunofluorescence Staining and Imaging

For immunofluorescence, tissues were fixed in 4% paraformaldehyde, washed with PBS, followed by overnight incubation with 30% (w/v) sucrose (Sigma-Aldrich) in PBS. Tissues were embedded in Optimal Cutting Temperature Compound (Tissue-Tek) and stored at −80° C. before sectioning on a cryostat (Leica). Slides were briefly rehydrated in PBS before permeabilization and blocking in CAS block (ThermoFisher) with 0.2% Triton X-100 (Sigma-Aldrich) followed by primary antibody staining at 4° C. overnight. When necessary, secondary antibody staining was performed at room temperature for 1 h. Sections were washed with PBS-Tween 0.1% before mounting with ProLong Diamond Antifade Mountant (ThermoFisher). Images were acquired on an Apotome microscope (Zeiss). The following antibodies were used: KRT8-Alx647 (clone EP1628Y, ab192468, Abcam), KRT5 Alx488 (clone EP1601Y, ab193894, Abcam), KRT15 (clone EPR1614Y, ab52816, Abcam), ASCL1 (ab74065, Abcam), AIRE (14-9534-82, eBioscience), SOX2 (AF2018, R&D Systems), KRT10-Alx647 (clone EP1607IHCY, ab194231, Abcam).

### Immunohistochemistry

Tissue was fixed in 4% paraformaldehyde (ThermoFisher), washed with PBS, and embedded in paraffin. Antigen retrieval was performed on rehydrated tissue by boiling sections in antigen retrieval Citra Solution (Biogenex). Sections were blocked for 30 min at room temperature using CAS-Block (ThermoFisher) with 0.2% Triton X-100 (Sigma-Aldrich), followed by incubation with primary antibody overnight at 4° C. Staining with biotinylated secondary antibody was performed for 1 h at room temperature. Slides were developed using an ABC kit (Vector labs) and DAB kit (Vector labs) and counterstained with haematoxylin. The following antibodies were used: anti-CFTR (Clone #24-1, R&D Systems), anti-Desmin (Clone D33, Dako), anti-synaptophysin (Clone Snp88, Biogenex).

### Mice

Rosa26-CAG-stopflox-tdTomato and Ascl1-cre-ERT2 (Kim, E. J., Ables, J. L., Dickel, L. K., Eisch, A. J. & Johnson, J. E. Ascl1 (Mash1) defines cells with long-term neurogenic potential in subgranular and subventricular zones in adult mouse brain. PLoS ONE 6, e18472 (2011)) mice were obtained from The Jackson Laboratory (JAX #007914 and #012882). ADIG mice have been described previously (Gardner, J. M. et al. Deletional Tolerance Mediated by Extrathymic Aire-Expressing Cells. Science 321, 843-847 (2008)). Mice were maintained in the University of California San Francisco (UCSF) specific pathogen-free animal facility in accordance with the guidelines established by the Institutional Animal Care and Use Committee (IACUC) and Laboratory Animal Resource Center. All experimental procedures were approved by the Laboratory Animal Resource Center at UCSF. Mice aged 12-15 weeks were used for the lineage tracing experiments. Tamoxifen (Sigma-Aldrich) was dissolved in corn oil (Sigma-Aldrich) and one 4 mg dose was administered by oral gavage with flexible plastic feeding tubes (Instech).

### Flow Cytometry and Antibodies

For lineage tracing experiments, single-cell suspensions were prepared as previously described (Miller, C. N. et al. Thymic tuft cells promote an IL-4-enriched medulla and shape thymocyte development. Nature 559, 627-631 (2018)). Briefly, mouse thymi were isolated, cleaned of fat and transferred to DMEM (UCSF Cell Culture Facility) containing 2% FBS (Atlanta Biologics) on ice. Thymi were minced with a razor blade and up to four thymi were pooled into a single digestion. Tissue pieces were moved to 15-ml tubes and vortexed briefly in digestion medium (DMEM containing 2% FBS, 100 μg/ml DNase I and 100 μg/ml liberase TM. Fragments were allowed to settle before removing the medium and replacing it with fresh digestion medium. Tubes were moved to a 37° C. water bath and fragments were digested with trituration with a glass Pasteur pipette every 6 min At 12 min, tubes were spun briefly to pellet undigested fragments and the supernatant was moved to 20 ml of 0.5% BSA (Sigma-Aldrich), 2 mM EDTA (TekNova) in PBS (MACS buffer) on ice to stop the enzymatic digestion. This was repeated twice for a total of three 12-min digestion cycles, or until there were no remaining tissue fragments. The single cell suspension was then pelleted and washed once in MACS buffer. Density-gradient centrifugation using a three-layer Percoll gradient (GE Healthcare) with specific gravities of 1.115, 1.065 and 1.0 was used to enrich for stromal cells. Cells isolated from the Percoll-light fraction, between the 1.065 and 1.0 layers, were resuspended in MACS buffer and counted. Cells were then incubated with Live/Dead Fixable Blue Dead Cell Stain (ThermoFisher) in PBS for 20 min at 4° C. followed by blocking with anti-mouse CD16/CD32 (24G2) (UCSF Hybridoma Core Facility) and 5% normal rat serum for 20 min at 4° C. Cells were then washed in FACS buffer and stained for surface markers for 30-45 min at 4° C. Flow cytometry data were collected on a LSRII Flow Cytometer (BD Biosciences) housed within the UCSF Single Cell Analysis Center, and analyzed using FlowJo software (TreeStar Software). The following antibodies were used: CD11c (N418), CD45 (30-F11), EpCAM (G8.8), I-Ab (25-9-17). Antibodies were purchased from Abcam, BioLegend, BD Biosciences, eBioscience or Miltenyi.

### Example 1. Single-Cell Profiling of Stromal Cells from Human Thymus

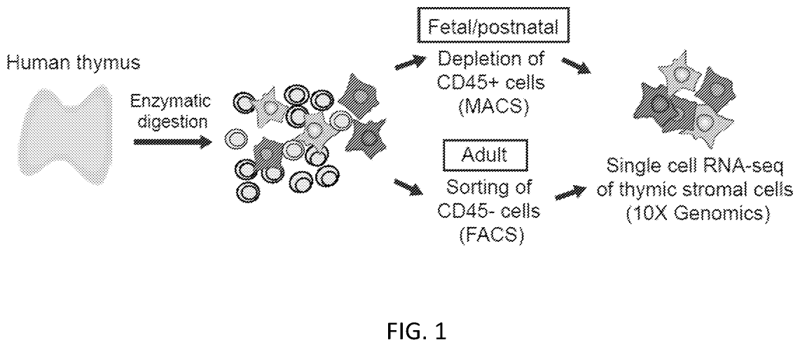

To identify the different cell types comprising the human thymic microenvironment, single-cell RNA sequencing (scRNA-seq) was performed using stromal cells isolated from fetal, postnatal, and adult thymic tissue (FIG. 1). FIG. 1 herein is also described as FIG. 1a of Bautista, J. L., et al. Nat Commun 12, 1096 (2021) (https://doi.org/10.1038/s41467-021-21346-6; the contents of which are herein incorporated by reference in its entirety). Stromal cells were obtained by enzymatic digestion of thymic tissue followed by depletion of CD45-positive immune cells using magnetic beads (MACS) or fluorescence-activated cell sorting (FACS) purification of CD45 negative cells. These procedures led to the enrichment of both EpCAM+/CD45− epithelial cells and EpCAM−/CD45− non-epithelial stromal cells. Cells isolated from two fetal (19 and 23 gestational weeks), two postnatal (6 day old and 10 month old), and one adult (25 year old) samples were analyzed using scRNA-seq.

Specifically, single cells were captured using the 10×Chromium microfluidics system (10×Genomics). The cells were encapsulated and barcoded cDNA libraries were prepared using the single-cell 3′ mRNA kit (v2 or v3; 10×Genomics). Single-cell libraries were sequenced using a NovaSeq 6000. The Cell Ranger software pipeline (10×Genomics, version 2.0.0, 2.1.1, or 3.0.2) was used to demultiplex cellular barcodes, map reads to the human genome (GRCh38), and transcriptome using the STAR aligner, and produce a matrix of gene counts versus cells. Single-cell data analysis was performed using SCANPY (Wolf, F. A., Angerer, P. & Theis, F. J. SCANPY: large-scale single-cell gene expression data analysis. Genome Biol. 19, 15-5 (2018)). Cell by gene count matrices of all samples were concatenated to a single matrix. Cells with 200-5000 detected genes and expressing <10% mitochondrial genes as well as genes expressed in >3 cells were retained (68008 cells total with an average of 4769 counts per cell). Counts were log transformed and total counts per cell were normalized. The dataset was filtered for highly variable genes (minimum mean=0.0125, maximum mean=3, and dispersion, 0.5 per gene) and variation caused by mitochondrial gene expression and cell cycle-dependent changes in gene expression were regressed out. Batch correction using “batch balanced k nearest neighbors” (BBKNN) analysis (Polański, K. et al. BBKNN: Fast Batch Alignment of Single Cell Transcriptomes. Bioinformatics 36, 411. (2019)) was applied to correct donor-specific effects. K-nearest neighbor graphs were constructed (n_neighbors=15) and clustering was performed using the Leiden algorithm with a resolution of 0.5. Employing an unsupervised graph-based clustering strategy, twelve stromal clusters were identified using known markers (Table 3). General epithelial markers EPCAM and KRT8 were used to identify epithelial clusters, with FOXN1, PSMB11, LY75, CLDN4, AIRE, IVL, NEUROD1, and MYOD1 identified as markers in specific subsets.

TABLE 3
Stromal cell clusters identified by scRNA-seq of
human thymic stromal cells

| Cluster | Marker |
|---|---|
| Epithelium-1 | EPCAM, KRT8 |
| Epithelium-2 | EPCAM, KRT8 |
| Epithelium-3 | EPCAM, KRT8 |
| Mesenchyme | PDGFRA, LUM, LAMA2 |
| Pericytes | PDGFRB, MCAM, |
| | CSPG4/NG2 |
| Vascular arterial | PECAM1, VEGFC, GJA4 |
| endothelial (Endo-1) | |
| Vascular venous | PECAM1, ACKR1, SELE, |
| endothelial (Endo-2) | APLNR |
| Vascular venous | PECAM1, ACKR1, SELE, |
| endothelial (Endo-3) | APLNR |
| Lymphatic endothelial | LYVE1, PROX1, CCL21 |
| (Endo-4) | |
| Red blood cells | GYPA, HBA1, HBG1 |
| Immune cell | PTPRC, CD3D, CD7 |
| Mesothelium | MSLN, UPK3B, PRG4 |

A list of stromal cell markers useful for identifying cell types within the human thymus is provided in Table 4. Ensembl gene identifiers (ENSG00000######) are provided for each marker.

TABLE 4
Stromal Cell Markers

| | |
|---|---|
| Epithelial Markers | Vascular Venous Endothelial |
| EPCAM (ENSG00000119888) | Markers |
| KRT8 (ENSG00000170421) | PECAM1 |
| FOXN1 (ENSG00000109101) | (ENSG00000261371) |
| PSMB11 (ENSG00000222028) | ACKR1 (ENSG00000213088) |
| LY75 (ENSG00000054219) | SELE (ENSG00000007908) |
| CLDN4 (ENSG00000189143) | APLNR (ENSG00000134817) |
| AIRE (ENSG00000160224) | Lymphatic Endothelial |
| IVL (ENSG00000163207) | Markers |
| NEUROD1 (ENSG00000162992) | LYVE1 (ENSG00000133800) |
| MYOD1 (ENSG00000129152) | PROX1 (ENSG00000117707) |
| Mesenchymal Markers | CCL21 (ENSG00000137077) |
| PDGFRA (ENSG00000134853) | Red Blood Cell Markers |
| LUM (ENSG00000139329) | GYPA (ENSG00000170180) |
| LAMA2 (ENSG00000196569) | HBA1 (ENSG00000206172) |
| Pericyte Markers | HBG1 (ENSG00000213934) |
| PDGFRB (ENSG00000113721) | Immune Cell Markers |
| MCAM (ENSG00000076706) | PTPRC (ENSG00000081237) |
| CSPG4/NG2 | CD3D (ENSG00000167286) |
| (ENSG00000173546) | CD7 (ENSG00000173762) |
| Vascular Arterial Endothelial | Mesothelial Markers |
| Markers | MSLN (ENSG00000102854 |
| PECAM1 (ENSG00000261371) | UPK3B (ENSG00000243566 |
| VEGFC (ENSG00000150630) | PRG4 (ENSG00000116690 |
| GJA4 (ENSG00000187513) | |

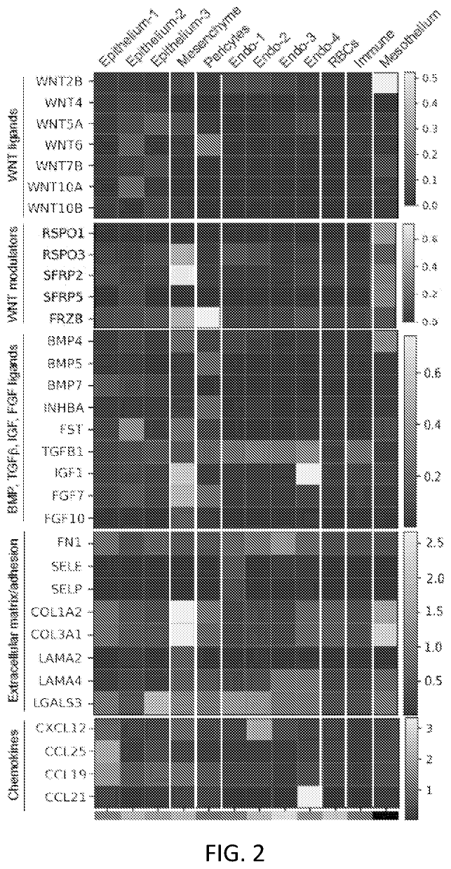

Studies in animal models have shown that neural crest, mesenchyme, and endothelial cells are important for the establishment of a thymic microenvironment that supports thymopoiesis through the production of soluble factors and cell-cell interactions (Kuratani, S. & Bockman, D. E. Anat. Rec. 228, 185-190 (1990); Jenkinson, W. E., Jenkinson, E. J. & Anderson, G. J. Exp. Med. 198, 325-332 (2003); Neves, H., Dupin, E., Parreira, L. & Le Douarin, N. M. Developmental Biology 361, 208-219 (2012); Sun, L. et al. Sci Rep 5, 14871 (2015); Wertheimer, T. et al. Sci Immunol 3, eaa12736 (2018)). The function and cell-type specificity of these soluble factors in human thymic development is however not well understood. To define the signals provided by human stromal cells, expression was assessed for soluble factors of the WNT, BMP, TGF beta, IGF, and FGF signaling pathways, which have been described as critical regulators of the development and function of TECs (Lepletier, A. et al. Cell Rep 27, 3887-3901.e4 (2019); Balciunaite, G. et al. Nature Immunology 3, 1102-1108 (2002); Gordon, J., Patel, S. R., Mishina, Y. & Manley, N. R. Developmental Biology 339, 141-154 (2010); Patel, S. R., Gordon, J., Mahbub, F., Blackburn, C. C. & Manley, N. R. Gene Expression Patterns 6, 794-799 (2006); Bleul, C. C. & Boehm, T. J. Immunol. 175, 5213-5221 (2005); Barsanti, M. et al. Eur. J. Immunol. 47, 291-304 (2017); Swann, J. B., Krauth, B., Happe, C. & Boehm, T. Sci Rep 7, 8492 (2017); Tsai, P. T., Lee, R. A. & Wu, H. Blood 102, 3947-3953 (2003)). The results revealed that mesenchymal cells expressed many ligands and regulators of these critical pathways, including WNT5A, RSPO3, SFRP2, IGF1, and FGF10 (FIG. 2). FIG. 2 herein is also described as FIG. 1f of Bautista, J. L., et al. Nat Commun 12, 1096 (2021) (https://doi.org/10.1038/s41467-021-21346-6; the contents of which are herein incorporated by reference in its entirety). Notably, BMP4, FGF7 (also known as KGF), and the secreted WNT inhibitor Frizzled Related Protein FRZB were expressed more frequently in postnatal and adult mesenchymal cells compared to fetal mesenchyme, suggesting that TEC differentiation and proliferation is differentially regulated by mesenchymal factors over time.

Most endothelial cells expressed TGFB1 and TGFBR2 while arterial and lymphatic subsets had high levels of chemokines known to promote homing of hematopoietic progenitors to the thymus (CXCL12 or CCL21), suggesting these chemokines are secreted by endothelial cells to regulate migration of, e.g., hematopoietic precursors. Subsets of cells also expressed WNT2B, WNT5A, RSPO3, BMP4, and IGF1. Endothelial cells also expressed extracellular matrix and adhesion molecules such as fibronectin (FN1) and LGALS3 (FIG. 2).

Epithelial cells and mesothelium were enriched for BMP7 and many WNT ligands (WNT4, WNT5A, WNT6, WNT7B, WNT10A, WNT10B) while mesothelium also expressed many WNT signaling modulators (WNT2B, RSPO1, RSPO3, SFRP2, SFRP5) and BMP4.

As for pericytes, they expressed FRZB as well as WNT6, BMP5, and FGF7. The gene encoding the subunits of Activin A (INHBA), which was recently shown to be important for TEC differentiation, was expressed almost exclusively by pericytes. In contrast, the activin antagonist follistatin (FST), which promotes TEPC maintenance and inhibits differentiation (Lepletier, A. et al. Cell Rep 27, 3887-3901.e4 (2019)), was found mostly in adult mesenchymal cells and a subset of epithelial cells.

Thus, human thymic mesenchymal cells, endothelial cells, and pericytes express many factors critical for TEC development. These results expose a previously unappreciated role of pericytes in regulating TEC differentiation through the secretion of activin signaling regulators. Overall, the results show that various different cell types support the thymic microenvironment. Epithelial cells, mesenchymal cells, pericytes, endothelial cells, red blood cells, immune cells, and mesothelial cells, including sub-groups thereof, are present and support the human thymic microenvironment and/or express factors that support the human thymic microenvironment.

### Example 2. Profiling of Human Thymic Epithelial Cells

To further define heterogeneity within the epithelial compartment, the three epithelial superclusters (Table 3, Epithelium-1, -2, and -3) were divided into nine distinct sub-clusters (Table 5).

TABLE 5
Sub-clustering of Epithelium-1, Epithelium-2, and
Epithelium-3 superclusters

| Epithelial | |
|---|---|
| sub-cluster | Marker |
| cTEC-hi | PSMB11, PRSS16, CCL25 |
| cTEC-lo | PSMB11, PRSS16, CCL25 (with lower |
| | levels of HLA class II, PSMB11, PRSS16, |
| | CCL25; increased KI67 + proliferating |
| | cells) |
| Immature | FOXN1, PAX9, SIX1, lacking cTEC/ |
| TEC | mTEC functional genes |
| mTEC-lo | CLDN4, lower levels of HLA class II, |
| | high levels of the chemokine CCL21 |
| Aire + mTEC | SPIB, AIRE, FEZF2, higher levels of |
| hi | HLA class II |
| Keratinocyte- | KRT1, IVL |
| like mTEC | |
| Neuroendo- | BEX1, NEUROD1 |
| crine | |
| Myoid | MYOD1, DES |
| Myelin | MPZ |

As used throughout this description, reference to “low” or “lower” levels of marker gene expression in a subpopulation of cells means about 1.5-fold lower, about 2-fold lower, about 2.5-fold lower, about 3-fold lower, about 3.5-fold lower, about 4-fold lower, about 4.5-fold lower, or about 5-fold lower, or lower than 5-fold lower than mean expression levels of the marker gene across a population of cells. For example, a cTEC-lo subpopulation may have lower levels of PSMB11 expression compared to average PSMB11 levels in the cTEC-hi subpopulation, meaning the cTEC-lo subpopulation has about 1.5-fold to about 5-fold lower expression levels of PSMB11 compared to the cTEC-hi subpopulation.

Similarly, throughout this description, reference to “high” or “higher” levels of marker gene expression in a subpopulation of cells means about 1.5-fold higher, about 2-fold higher, about 2.5-fold higher, about 3-fold higher, about 3.5-fold higher, about 4-fold higher, about 4.5-fold higher, or about 5-fold higher, or higher than 5-fold higher than mean expression levels of the marker gene across a population of cells. For example, a mTEC-lo subpopulation may have higher levels of CCL21 expression compared to average CCL21 levels in other epithelial populations, meaning the mTEC-lo subpopulation has about 1.5-fold to about 5-fold higher expression levels of CCL21 compared to other epithelial populations such as mTEC-hi.

The sub-clusters were annotated based on a combination of known TEC markers and a list of differentially expressed genes including FOXN1, PAX9, SIX1, HLA-DQB1, PSMB11, CLDN4, CCL21, PSMB11, CLDN4, CCL21, SPIB, AIRE, FEZF2, IVL, NEUROD1, MYOD1, MPZ, and MKI67. Two sub-clusters expressed genes characteristic of cTECs (PSMB11, PRSS16, CCL25). Cells in the cTEC-lo sub-cluster expressed lower levels of functional genes (HLA class II, PSMB11, PRSS16, CCL25) and contained more KI67+ proliferating cells. Genes characteristic of mTECs were detected in three sub-clusters corresponding to mTEC-lo (CLDN4, lower levels of HLA class II), mTECs-hi (SPIB, AIRE, FEZF2, higher levels of HLA class II), and keratinocyte-like mTECs (KRT1, IVL). Cells in the mTEC-lo sub-cluster expressed high levels of the chemokine CCL21, reminiscent of the CCL21-expressing mTEC-lo/jTEC population described in mice (Lkhagvasuren, E., Sakata, M., Ohigashi, I. & Takahama, Y. The Journal of Immunology 190, 5110-5117 (2013); Onder, L. et al. Eur. J. Immunol. 45, 2218-2231 (2015); Miragaia, R. J. et al. Sci Rep 8, 685 (2018); Michel, C. et al. The Journal of Immunology 199, 3488-3503 (2017)). One sub-cluster of cells was identified as immature TEC, which express canonical TEC identity genes (FOXN1, PAX9, SIX1) but lacked functional genes characteristic of cTECs or mTECs. These immature cells are found in all samples and possibly represent progenitors that are not committed to a specific lineage or cells that have lost their differentiated phenotype. Three more sub-clusters were identified as neuroendocrine (BEX1, NEUROD1), muscle-like myoid (MYOD1, DES), and myelin+ epithelial cells (MPZ).

### Example 3. Identification of Novel TEC Markers

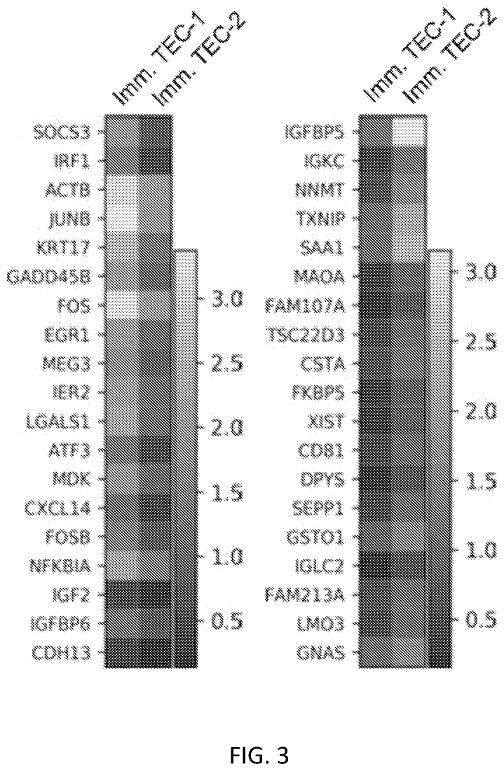

Additional markers of TEC subsets were further analyzed with a particular focus on immature TECs. The zinc finger protein ZBED2, a transcription factor without a murine counterpart that has been recently linked to the maintenance of the basal state in human keratinocytes, was identified as a gene highly expressed in immature TECs and cTECs. Genes regulating TGF-β signaling, including TDGF1 (also known as CRIPTO) and CTGF as well as IGF signaling modulators (IGFBP5 and IGFBP6) were also enriched in immature TECs and cTECs. In addition, an atypical cadherin gene (CDH13) was identified as a cell surface marker that could potentially be used in combination with cTEC surface markers to separate immature TECs from other TEC subsets. Two subpopulations were identified (immature TEC-1 and immature TEC-2) that expressed distinct markers (FIG. 3). FIG. 3 herein is also described as FIG. 3c of Bautista, J. L., et al. Nat Commun 12, 1096 (2021) (https://doi.org/10.1038/s41467-021-21346-6; the contents of which are herein incorporated by reference in its entirety). Interestingly, the expression of some genes enriched in immature TEC-2 (IGFBP5, NNMT, MAOA, DPYS, FKBP5, GLUL) was markedly higher in adult cells compared to fetal and postnatal tissues.

While the expression of many cytokeratins has been extensively studied and used as markers of specific TEC subsets, the expression of KRT15 in the thymus has not been reported before. This cytokeratin is particularly interesting since it is found in multipotent progenitor populations in the hair follicle, esophageal epithelium, and small intestine (Lyle, S. et al. J. Cell. Sci. 111 (Pt 21), 3179-3188 (1998); Giroux, V. et al. J. Clin. Invest. 127, 2378-2391 (2017); Giroux, V. et al. Stem Cell Reports 10, 1947-1958 (2018)). KRT15 was highly expressed in mTEC-lo but was also detected in immature TECs and its expression increased over time. Immunofluorescence confirmed that KRT8+/KRT5+ cells found at the cortico-medullary junction, which potentially mark immature TECs, expressed low level of KRT15 while KRT15-hi cells were found in the medulla and co-expressed KRT5, likely marking CCL21+ mTEC-lo (data not shown). Most TECs isolated from adult tissue expressed a combination of KRT8, KRT5, and KRT15.

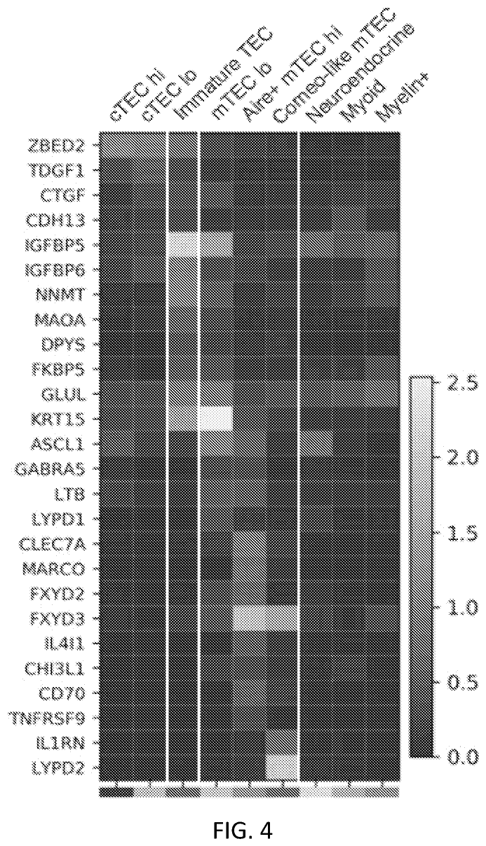

In addition to immature TEC markers, genes enriched in mTEC-lo (GABRA5, LYPD1), mTEC-hi (CLEC7A, MARCO, FXYD2, FXYD3, IL4I1, CHI3L1, CD70/CD27L, TNFRSF9), or keratinocyte-like mTECs (FXYD3, IL1RN, LYPD2) were identified (FIG. 4). FIG. 4 herein is also described as FIG. 4a of Bautista, J. L., et al. Nat Commun 12, 1096 (2021) (https://doi.org/10.1038/s41467-021-21346-6; the contents of which are herein incorporated by reference in its entirety). Notably, some of the genes marking AIRE+ cells code for cell surface proteins that could allow sorting of this population from human thymus. The proneural basic helix-loop-helix (bHLH) transcription factor Achaete-scute complex 1 (ASCL1) was also enriched in multiple epithelial subsets, including cTEC-hi, mTEC-lo, a subset of AIRE+ mTEC-hi, and neuroendocrine cells (data not shown). The role of this chromatin remodeling factor is well characterized in the brain, where it is expressed in dividing neural progenitors and promotes their proliferation, specification, and differentiation into neurons (Bertrand, N., Castro, D. S. & Guillemot, F. Nat. Rev. Neurosci. 3, 517-530 (2002); Castro, D. S. et al. Genes & Development 25, 930-945 (2011)). ASCL1 has also been shown to play a role in the development of neuroendocrine cells in the lung (Borges, M. et al. Nature 386, 852-855 (1997)). Expression of ASCL1 in the medulla of fetal and postnatal thymus was confirmed by immunofluorescence while expression was also observed in the cortex of fetal but not postnatal tissue (data not shown). Co-expression of ASCL1 with KRT15 was detected in a subset of medullary cells, likely representing CCL21+ mTEC-lo while KRT15 negative cells likely mark AIRE+ mTEC-hi. Immunofluorescence staining confirmed the presence of cells that co-expressed ASCL1 and AIRE (data not shown). ASCL1 target genes such as INSM1, DLL3, HES6, ST3GAL5, LYPD1, and POU4F1 were detected in TECs, suggesting activity of ASCL1 rather than expression as part of a promiscuous gene expression program.

To assess whether ASCL1 transcription factor also marks a pool of progenitor cells in the thymus, an in vivo genetic lineage tracing experiment using a fluorescent reporter system (Ascl1creERT2; Rosa26CAGstopflox-tdTomato) was performed. In this model, tamoxifen treatment permanently labeled ASCL1-expressing cells and their progeny, allowing to distinguish between long-lived progenitor cells (labeled cells can be found long after Cre induction) and transit-amplifying cells (reporter expressing cells diminish over time). These mice were also crossed to an Aire GFP-reporter line (Gardner, J. M. et al. Science 321, 843-847 (2008)) to facilitate quantification of Aire expression by flow cytometry. Mice were injected once with tamoxifen and thymi were harvested at 36 h and 5 weeks post-tamoxifen to analyze expression of the fluorescent reporters. 19% of TECs were labeled with the Ascl1 lineage tracing after 36 hours while the number dropped to 5% after 5 weeks. The percentage of Aire-expressing cells labeled with the Ascl1-lineage tracing also declined over time, implying that cells that maintain the pool of Aire+ mTEC-hi are not expressing Ascl1. These data thus suggest that Ascl1+ mTEC-lo and Ascl1+ mTEC-hi are likely replenished from a pool of Ascl1-negative cells.

### Example 4. Lineage Decisions within the Thymic Epithelial Compartment

To better understand the relationship between the different epithelial subsets, pseudotime analysis was performed using RNA Velocity (Yano, M. et al. Aire controls the differentiation program of thymic epithelial cells in the medulla for the establishment of self-tolerance. Journal of Experimental Medicine 205, 2827-2838 (2008)). This method, which considers both spliced and unspliced mRNA counts to estimate how mRNA levels evolve over time, can be used to predict potential directionality of transitions between cell states. Since murine TEC development differs significantly between embryonic and postnatal tissue, fetal, postnatal, and adult samples were analyzed separately. Analysis showed that cTECs-lo give rise to cTECs-hi in both fetal and postnatal tissues. The analysis also implied that cTECs-lo can give rise to immature TECs in fetal samples while in the postnatal tissue, this relationship seems to be inverted with immature TEC transitioning back to cTEC-lo. Immature TECs were recovered from the adult tissue but there was almost no cTECs detected, suggesting that there is an accumulation of immature TECs to the detriment of functional cTECs in older tissue. The analysis predicts that AIRE+ mTEC-hi precedes both CCL21+ mTEC-lo and keratinocytes-like mTEC-lo in fetal tissues while AIRE+ mTEC-hi seem to give rise to keratinocytes-like mTEC only in postnatal tissues. Thus, CCL21+ mTEC-lo can arise from AIRE+ mTECs in fetal tissue.

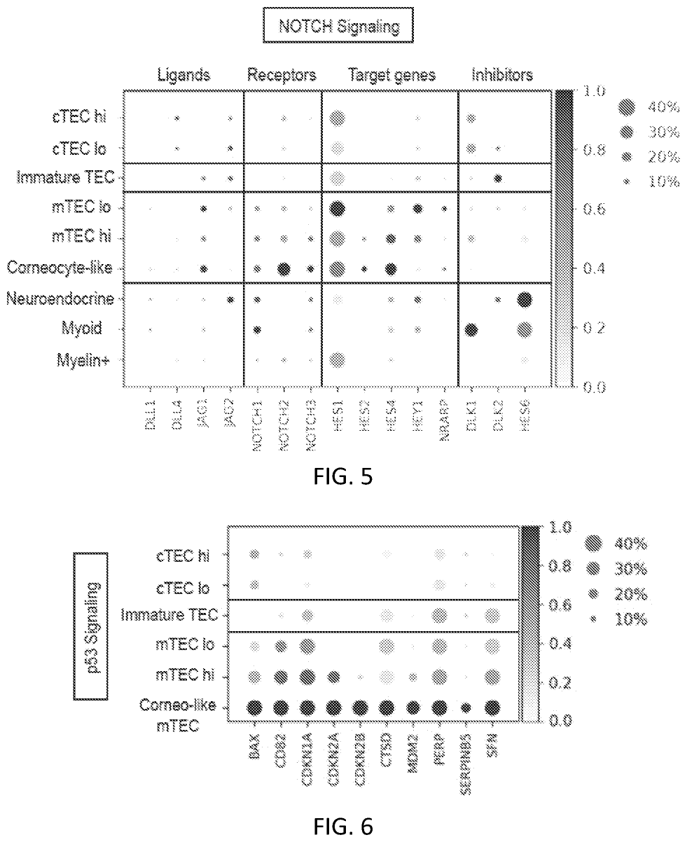

The analysis confirmed that the epithelial compartment is comprised of many different subsets. Analysis of differentially expressed genes between all epithelial clusters identified the Notch target gene HES1 and Notch inhibitor HES6 as being enriched in mTEC-lo and Neuroendocrine/Myoid clusters, respectively. Given the critical role of Notch signaling in regulating lineage choice in other tissues, expression of genes involved in this pathway, including ligands, receptors, target genes, and inhibitors, was examined DLL4, a key ligand promoting Notch1-dependent T cell specification and maturation, and JAG2 were the main ligands expressed in cTECs while JAG1 was detected in immature TECs and mTECs (FIG. 5). FIG. 5 herein is also described as FIG. 5b of Bautista, J. L., et al. Nat Commun 12, 1096 (2021) (https://doi.org/10.1038/s41467-021-21346-6; the contents of which are herein incorporated by reference in its entirety). Importantly, three of the receptors (NOTCH1, NOTCH2, NOTCH3) and some of their target genes (HES1, HES2, HES4, HEY1, and NRARP) were expressed at higher levels in mTECs, indicating that Notch signaling is more active in mTECs than cTECs. The Notch inhibitor DLK1 was also detected in cTECs while DLK2 was found in immature TECs, implying that Notch signaling is reduced in these cells. Finally, expression of HES6, a negative regulator of HES1, was considerably higher in neuroendocrine and myoid cells, indicating that Notch activity is actively blocked in these epithelial subsets. These results demonstrate that, in addition to its critical role in T cell specification, the regulation of Notch signaling affects cell fate outcomes in the epithelial compartment. Thus, factors in the Notch signaling pathway can be used to direct and/or confirm thymic cell differentiation.

To further define TEC specification, pathways that have been shown to regulate the development of CCL21+ mTEC-lo and AIRE+ mTEC-hi in mice were analyzed. The maintenance of murine AIRE+ mTECs-hi cells is mediated by TNF receptor superfamily signals, including receptor activator for NF-κB (RANK), CD4039-42, and osteoprotegerin (OPG), which acts as a decoy receptor for RANKL43 while CCL21+ mTECs-lo depend on LTBR signaling. Expression of a subset of these receptors was detected in CCL21+ mTEC-lo and keratinocyte-like mTECs (LTBR), mTEC-hi (RANK/TNFRSF11A, OPG/TNFRSF11B), or mTEC-lo and mTEC-hi (CD40), confirming that similar pathways are likely controlling the differentiation of mTEC-hi in humans Intriguingly, other TNF receptors were also found in mTECs-hi (TNFRSF4 (OX40) and TNFRSF9 (4-1BB)) as well as some TNF ligands in mTEC-lo (LTB) or mTEC-hi (LTB and CD70). Given the importance of LTBR signaling for the development of CCL21+ mTECs, FEZF2+ mTECs44, and keratinocyte-like mTECs, the observation that mTECs-lo themselves as well as mTECs-hi express the ligand LTB suggest that, in addition to signals from thymocytes, the mTEC compartment itself can regulate the differentiation of these TEC subsets. Taken together, the analysis revealed novel regulators of cell fate commitment in the epithelial compartment. Tuft cells, ionocytes, and ciliated cells are present in the human thymic medulla.

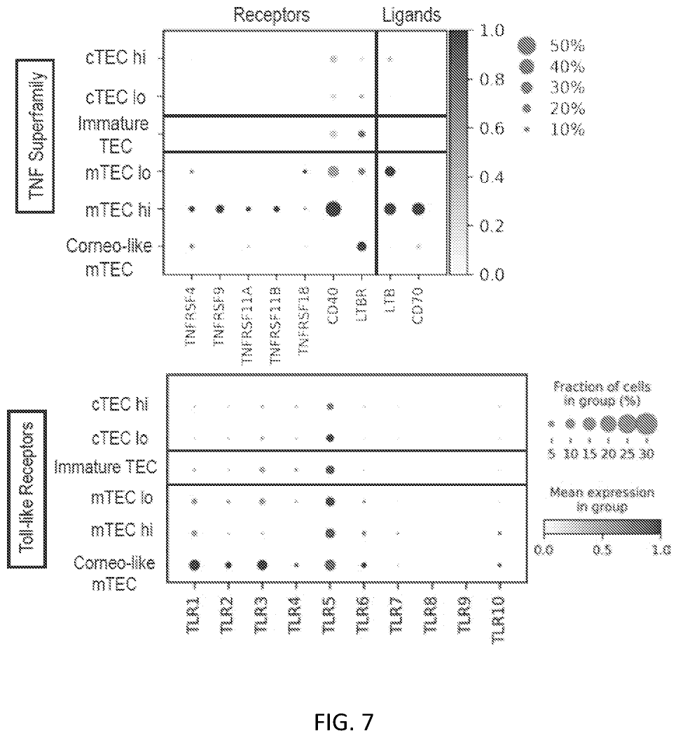

Genes associated with the p53 signaling (PERP, SFN, CTSD, CDKN2A, 302 CDKN2B) (FIG. 6) as well as many TNF Superfamily (FIG. 7, top panel) and Toll-like receptors (TLR1-6, TLR10) (FIG. 7, bottom panel) were identified as upregulated in corneocyte-like mTECs. FIG. 6 herein is also described as FIG. 5e of Bautista, J. L., et al. Nat Commun 12, 1096 (2021) (https://doi.org/10.1038/s41467-021-21346-6; the contents of which are herein incorporated by reference in its entirety). FIG. 7 top panel and bottom panel herein are also described as FIG. 5d and FIG. 5f of Bautista, J. L., et al. Nat Commun 12, 1096 (2021) (https://doi.org/10.1038/s41467-021-21346-6; the contents of which are herein incorporated by reference in its entirety). The data suggest a corneocyte-like/post-AIRE mTEC subset with high levels of p53 activity as dependent on the p53 signaling pathway. As for Toll-like receptors, it is possible that this pathway regulates the differentiation of mTEC-hi cells into involucrin+ post-AIRE cells. Taken together, the findings revealed important information on the process of cell fate commitment in the epithelial compartment and factors involved in these pathways can be used to direct and/or confirm thymic cell differentiation.

To better understand the heterogeneity of the medullary compartment, characterize rare medullary epithelial subsets, and gain insights into the relationship between mTECs and other epithelial subsets, mTECs, neuroendocrine, myoid, and myelin expressing cells were re-clustered and resolution was increased to obtain eight clusters. Newly identified sub-clusters are shown in Table 6.

TABLE 6
Re-clustered mTECs, neuroendocrine, myoid, and
myelin expressing cells

| Epithelial sub- | |
|---|---|
| cluster | Marker |
| Tuft/Ionocytes | GNB3, TRPM5, GNAT3, PLCB2, OVOL3, |
| | POU2F3, FOXI1, ASCL3, CFTR, CLCNKB |
| Ciliated | ATOH1, GFI1, LHX3, FOXJ1 |
| Myelin+ | SOX10, MPZ, MBP, S100A1 |

A previously unreported population of ciliated cells (positive for ATOH1, GFI1, LHX3, FOXJ1) was identified. Myelin+ cells were found to closely resemble Schwann cells (SOX10, MPZ, MBP, S100A1). A cluster that contained cells with a signature characteristic of chemosensory tuft cells recently identified in the murine thymus (GNB3, TRPM5, GNAT3, PLCB2, OVOL3, POU2F3) was also identified (see Miller, C. N. et al. Nature 559, 627-631 (2018); Bornstein, C. et al. Nature 559, 622-626 (2018)). Additionally, cells with a signature reminiscent of the ionocyte population present in lung tissue (FOXI1, ASCL3, CFTR, CLCNKB) were identified (see Montoro, D. T. et al. Nature 560, 319-324 (2018); Plasschaert, L. W. et al. Nature 560, 377-381 (2018)). Ciliated cells and ionocytes were found in all samples while myelin+ were detected in fetal and adult samples only. The presence of ionocytes in the thymus was confirmed by immunofluorescence for KRT8+/CFTR+ cells in the human thymic medulla of both fetal and postnatal tissue (data not shown). Ionocytes were found as isolated cells in the medulla or as part of Hassall's corpuscles. In addition to ionocytes and thymic tuft cells, neuroendocrine cells and a subset of myoid cells were also detected in close proximity to Hassall's corpuscles (data not shown). These data thus demonstrated that Hassall's corpuscles are complex structures that are more heterogeneous than previously appreciated. Analysis of differentially expressed genes among medullary cells identified the transcription factor SOX2 as being highly expressed in ciliated cells. In addition, expression of SOX2 was detected in keratinocytes-like mTECs, neuroendocrine, and myelin+ cells.

A role for this transcription factor in the postnatal thymus has never been reported. Immunofluorescence analysis confirmed that SOX2 was expressed in Hassall's corpuscles as well as in a few isolated cells scattered in the medulla (data not shown). SOX2 staining was observed in KRT8+ cells as well as in cells expressing KRT5 and/or KRT10, confirming expression in different subsets of medullary epithelial cells.

Taken together, the single-cell profiling of medullary epithelial cells revealed the presence of new cell types in the thymic medulla, further highlighting the cellular complexity of the human thymic stroma.

### Example 5. Characterization of Tissue-Specific Antigen Expression by Human TECs

Although it is clear that loss-of-function mutations in the human AIRE gene result in a multiorgan autoimmune disease known as autoimmune polyendocrinopathy candidiasis ectodermal dystrophy (APECED) or autoimmune polyglandular syndrome type 1 (APS-1) (Finnish-German APECED Consortium. An autoimmune disease, APECED, caused by mutations in a novel gene featuring two PHD-type zinc-finger domains. Nat. Genet. 17, 399-403 (1997); Nagamine, K. et al. Nat. Genet. 17, 393-398 (1997)), direct evidence of TSA expression in human AIRE+ mTECs is lacking. To assess if AIRE+ mTECs expressed higher levels of TSAs than other epithelial subsets, a TSA score was calculated by averaging expression of a list of tissue-restricted genes (compiled by Sansom, S. N. et al. Genome Res. 24, 1918-1931 (2014)) and subtracting the average expression of a reference set of genes. The results were visualized using UMAP (data not shown). This analysis confirmed that TSA expression was particularly enriched in human AIRE+mTEC-hi cells when compared to other epithelial subsets. A similar approach was used to analyze the expression of antigens known to elicit auto-antibodies in APS-1 patients (Constantine, G. M. & Lionakis, M. S. Immunol. Rev. 287, 103-120 (2019)), which are predicted to be AIRE-dependent genes in humans. This analysis confirmed that APS-1 relevant genes were enriched in AIRE+ mTECs. In addition, visualization of individual genes using UMAP revealed which antigens were mostly detected in AIRE+/post-AIRE keratinocyte-like mTECs (IL6, IL17F, IL22, CASR, TG, TPO, BPIFB1, DDC, TPH1, ATP4A, ATP4B, HDC, TGM4, TH). In contrast, other genes were mainly expressed in the neuroendocrine or myelin+ clusters (DEFA5, SOX10, MPZ). This analysis thus provides new evidence that AIRE-expressing cells play a critical role in the development of the APECED/APS-1 disease.

To better understand the role of TEC subsets in the induction of immune tolerance in humans, expression of antigens that have been shown to play a role in organ specific autoimmune diseases was analyzed. Antigens relevant to type 1 diabetes (T1D) like insulin (INS) and IAPP were found in rare AIRE+/post-AIRE cells while IA-2 (PTPRN) was detected in a few neuroendocrine cells. These data thus confirm that, similar to the murine thymus, expression of insulin in the human thymus is likely AIRE-dependent. In contrast, genes coding for the acetylcholine receptor (CHRNA1), the muscle antigen titin (TTN), and MUSK, which are associated with the neuromuscular autoimmune disease myasthenia gravis, were predominantly found in the neuroendocrine and myoid subsets (data not shown). These data indicate that distinct sources of disease-relevant antigens exist in the human thymic medulla, raising the possibility that multiple subsets of epithelial cells participate in the induction of immune tolerance.

### Example 6. Transplantation of Reaggregated Thymus

Purified MTS24+ embryonic TECs (R2 fraction) were reaggregated and transplanted under the kidney capsule. 8 weeks later, the cells had formed a structured functional thymus with clearly separated cortical and medullary regions (MTS10+) and blood vessels (CD31). See Gill, Jason, et al. “Generation of a complete thymic microenvironment by MTS24+ thymic epithelial cells.” Nature immunology 3.7 (2002): 635-642, incorporated herein by reference in its entirety.

Purified CD205+/CD40− embryonic TECs were reaggregated and transplanted under the kidney capsule. 6-8 weeks later, the cells had formed a structured thymus with clearly separated cortical and medullary (ERTR5) regions and Aire expression. See Generation of both cortical and Aire+ medullary thymic epithelial compartments from CD205+ progenitors. See Baik, Song, et al. “Generation of both cortical and Aire+ medullary thymic epithelial compartments from CD 205+ progenitors.” European journal of immunology 43.3 (2013): 589-594, incorporated by reference herein in its entirety. Purified CD205+CD40− E15 TECs (typically, 1×10⁵ cells) were used to prepare reaggregated thymus organ culture (RTOC). Intact RTOC were transplanted under the kidney capsule of wild type mice and harvested after 6-8 weeks. RTOCs were made from mixtures of thymic stroma in the presence of 2′-deoxyguanosine (dGuo) and either CD4+8+ thymocytes or CD4+3−LTi cells at a 1:1 ratio.

Different populations of cells were sorted and injected in a fetal thymus prior to kidney capsule transplantation. 4 weeks later, the sorted cells (GFP+) co-expressed markers of cortical TECs (DEC205) and mTECs (Aire). See Wong, Kahlia, et al. “Multilineage potential and self-renewal define an epithelial progenitor cell population in the adult thymus.” Cell reports 8.4 (2014): 1198-1209, incorporated by reference herein in its entirety. RTOCs were performed as recently described in Seach, Natalie, et al. “Double positive thymocytes select mucosal-associated invariant T cells.” The Journal of Immunology 191.12 (2013): 6002-6009, incorporated herein by reference in its entirety. Briefly, E14.5 thymic lobes were enzymatically digested and reaggregated with GFP+ adult TEC subsets at a ratio of 5:1. Typically, between 7.5×10\hat{ }4 and 1×10\hat{ }5 adult TECs were added per RTOC. Reaggregates were incubated over-night (18 hr) at 37 C (5% CO2) before engraftment under the kidney capsule of B6 adult male recipients. Grafts were harvested between 1 and 12 weeks and enzymatically digested for FACS analysis or snap frozen for immunofluorescence (IF).

### Example 7. Transplantation of hESC Derived TEPs in NSG Mice

Luciferase+ hESCs (e.g., MEL1-INS-GFP or MEL1-FOXN1-GFP cells) were differentiated to TEPs in Matrigel according to the above-described protocol and subsequently transplanted under kidney capsule of NSG mice. Survival of hESCs was quantified over time in mice using bioluminescence. Kidneys were harvested after 4-6 weeks. TEPs demonstrated good survival in vivo after 4-6 weeks. Overgrowth of non-epithelial cell types was observed.

### Example 8. Enrichment and Reaggregation of EPCAM+ Cells

To reduce overgrowth of non-epithelial cells in transplanted TEPs in vivo, EPCAM+ cells were enriched and reaggregated prior to transplant. EPCAM+ cells were enriched using magnetic bead-based purification. A purified cell population was confirmed by detecting TEP cell-identifying marker FOXN1. Reaggregation was accomplished in two ways: using an air-to-liquid approach comprising culturing cells on a porous membrane overlaying a liquid growth medium and using AggreWell plates.

### Example 9. Incorporation of Other Cell Types into the TEP Reaggregates

To recapitulate the complexity of the thymic microenvironment in TEP reaggregates, different types of support cells found in the human thymic microenvironment were identified using single cell RNA sequencing (scRNA-seq). Fetal and pediatric thymic tissue were compared. Fetal or postnatal human thymus tissue was enzymatically digested and CD45+ cells were depleted using magnetic-activated cell sorting (MACS). Single cell RNAseq of non-immune cells was performed, and cell type based on marker gene expression was determined in human fetal thymus at 19 weeks and 23 weeks, and human postnatal thymus at 8 days and 10 months old. Cell types identified included lymphatic endothelium, vascular endothelium, epithelium (including cTECs and mTECs), immune cells, mesenchyme, pericytes, and red blood cells. A roadmap of human thymic development was delineated from the RNAseq output as follows: cTEC lineage cells to bipotent TEPs; bipotent TEPs to committed mTEC progenitors; committed mTEC progenitors to immature mTECs; immature mTECs to mature TECs; mature TECs to post-Aire mTECs. Tuft cells and other TEC subsets were identified.

Different combinations of cells were reaggregated with hESC-TEPs and transplanted in humanized mice according to the protocols outlined above. In short, purified EPCAM+ cells were reaggregated with other support cell types (e.g., fibroblasts, endothelial cells, immune cells). Athymic NSG-FOXN1 null or thymectomized mice were subjected to sub-lethal irradiation and injection of human CD34+ human stem cells. Reaggregated cells were transplanted under kidney capsule. Mice were monitored for the presence of human T cells in peripheral blood. Grafts were harvested and analyzed by flow cytometry and immunofluorescence for the presence of human thymocytes and TECs.

### Example 10. Culturing of Thymic Epithelial Cells from Pluripotent Stem Cells In Vitro

Thymic epithelial cells are cultured in vitro according to the protocol set forth above and in Table 2 and FIG. 2. In brief, on Day 1 pluripotent stem (PS) cells are cultured in RPMI medium with 0.2% KSR/FBS in the presence of Activin A (about 100 ng/ml) and Wnt3a (about 50 ng/ml). On Day 2-3, the medium is replaced for RPMI with 0.2% KSR/FBS and Activin A (about 100 ng/ml) and ITS (at a 1:1000 dilution). Day 4 the medium is changed for RPMI having B27 (0.5×), Activin A (about 100 ng/ml), retinoic acid (about 0.25 uM), and LDN (about 250 nM). Day 5-6, medium is changed for DMEM medium containing B27 (0.5×), BMP4 (about 50 ng/ml), retinoic acid (about 0.25 uM), FGF8 (about 50 ng/ml), TGFβi IV (about 2.5 uM), IWP2 (about 5 uM), and ITS (1:1000). Day 7-12, medium is changed for DMEM medium containing B27 (0.5×), BMP4 (about 50 ng/ml), FGF8 (about 50 ng/ml), TGFβi IV (about 2.5 uM), and ITS (1:200). Day 13+, medium is changed for DMEM medium containing B27 (0.5×), KGF (about 50 ng/ml), ITS (1:200), heparin (about 10 ug/ml), hydrocortisone (about 0.5 ug/ml), and T3 (about 200 nM).

Cells are analyzed by FACS or MACS at one or more stage of differentiation to assess and sort and/or enrich based on expression of one or more marker genes.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A method for generating thymic epithelial progenitor (TEP) cells in vitro, the method comprising:
culturing a population of cells in a first medium comprising an activator of bone morphogenetic protein (BMP) signaling, an activator of fibroblast growth factor (FGF) signaling, and an inhibitor of transforming growth factor-β(TGF-β) signaling, and
further culturing the population of cells to induce further maturation of the TEP cells in vitro, wherein the further culturing comprises culturing the population of cells in a second medium comprising keratinocyte growth factor (KGF), heparin, and hydrocortisone.

2. The method of claim 1, wherein the second medium further comprises a triiodo-L-thyronine (T3) supplement.

3. The method of claim 1 or 2, wherein the second medium further comprises an insulin-transferrin-selenium (ITS) supplement.

4. The method of any one of claims 1-3, wherein the first and/or second medium further comprises a B27 supplement.

5. The method of any one of claims 1-4, wherein the population of cells comprises one or more of definitive endodermal (DE) cells, anterior foregut endodermal (AFE) cells, ventral pharyngeal endodermal (VPE) cells, and TEP cells.

6. The method of any one of claims 1-4, wherein the further culturing the population of cells to induce further maturation of the TEP cells in vitro comprises further culturing the population of cells for up to 14 days.

7. The method of any one of claims 1-6, wherein the further culturing the population of cells to induce further maturation of the TEP cells in vitro comprises transferring the population of cells to an extracellular matrix-based medium such as Matrigel.

8. The method of any one of claims 1-6, wherein the first and/or second medium is a liquid medium and the culture conditions comprise suspension culture.

9. The method of claim 8, wherein the first and/or second medium is a minimum essential medium or Dulbecco's minimum essential medium (DMEM).

10. The method of any one of claims 1-9, further comprising transplanting the TEP cells to a subject, wherein the further culturing the population of cells to induce further maturation of the TEP cells in vitro gives rise to subpopulations of thymic epithelial cells (TECs) after transplantation in vivo.

11. The method of claim 10, wherein the subpopulations of TECs comprise one or more of cortical TEC (cTEC) lineage cells, bipotent TEP cells, committed medullary TEC (mTEC) progenitor cells, immature mTECs, mature mTECs, post-AIRE mTECs, tuft cells, neuroendocrine cells, ionocytes, ciliated cells, myelin expressing cells, and/or myoid cells.

12. The method of any one of claims 1-11, further comprising transferring the population of cells to an air-liquid interface culture system before transplanting.

13. The method of any one of claims 1-12, further comprising reaggregating the cells to form a reaggregate before transplanting.

14. The method of any one of claims 1-13, further comprising reducing or eliminating non-epithelial cells from the culture of differentiated TEP cells.

15. The method of claim 14, comprising enriching for EPCAM+ TEP cells.

16. The method of any one of claims 1-15, comprising adjusting culture conditions or combining cell types in culture to recapitulate a thymic microenvironment.

17. The method of claim 16, wherein recapitulating the thymic microenvironment comprises culturing TEP cells under conditions sufficient to support survival of lymphatic endothelium cells, vascular endothelium cells, immune cells, mesenchymal cells, pericytes, red blood cells, or combinations thereof.

18. The method of claim 16, wherein recapitulating the thymic microenvironment comprises culturing TEP cells under conditions sufficient to support differentiation of TECs and subpopulations thereof.

19. The method of claim 18, wherein the TECs and subpopulations thereof comprise cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

20. The method of any one of claims 1-19, wherein the method comprises culturing definitive endodermal (DE) cells in the first medium comprising an activator of retinoic acid receptor, an activator of BMP signaling, an activator of FGF signaling, and an inhibitor of TGF-β signaling.

21. The method of claim 20, wherein the DE cells are differentiated in an initial cell culture medium comprising an inhibitor of BMP signaling in advance of the first medium comprising the activator of BMP signaling.

22. The method of claim 20 or 21, wherein an inhibitor of Wnt signaling is introduced into the first medium.

23. The method of any one of claims 20-22, wherein the method comprises culturing anterior foregut endodermal (AFE) cells produced by said culturing of the DE cells, wherein said culturing of the AFE cells is in the first medium comprising an activator of BMP signaling, an activator of FGF signaling, and an inhibitor of TGF-β signaling.

24. The method of claim 23, wherein the method comprises culturing ventral pharyngeal endodermal (VPE) cells produced by said culturing of the AFE cells, wherein said culturing of the VPE cells is in the first medium comprising an activator of BMP signaling, an activator of FGF signaling, and an inhibitor of TGF-β signaling.

25. The method of claim 23 or 24, wherein the first medium is not supplemented with an activator of retinoic acid receptor signaling.

26. The method of any one of claims 1-25, wherein the starting cells are obtained from pluripotent stem (PS) cells.

27. The method of claim 26, wherein the PS cells are embryonic stem cells, embryonic germ cells, or induced pluripotent stem (iPS) cells.

28. The method of claim 26 or 27, wherein the PS cells are primate pluripotent stem cells (pPS) cells.

29. The method of any one of claims 26-28, wherein the PS cells are human pluripotent stem (hPS) cells.

30. A method for generating TEP cells in vitro, the method comprising culturing a population of cells comprising anterior foregut endodermal (AFE) cells in a first cell culture medium comprising an activator of BMP signaling, an activator of FGF signaling, and an inhibitor of TGF-β signaling to produce TEP cells.

31. The method of claim 30, wherein the first cell culture medium is substantially free of at least one of: an activator of retinoic acid receptor signaling, an activator of Wnt signaling, and an inhibitor of hedgehog signaling.

32. A method for generating TEP cells in vitro, the method comprising culturing a population of cells comprising definitive endodermal (DE) cells in a first cell culture medium comprising an activator of BMP signaling, an activator of retinoic acid receptor signaling, an activator of FGF signaling, an inhibitor of TGF-β signaling, and an inhibitor of Wnt signaling to produce a population of cells comprising AFE cells, and culturing the population of AFE cells in a cell culture medium comprising an activator of BMP signaling, an activator of FGF signaling, an inhibitor of TGF-β signaling, and substantially free of an inhibitor of Wnt signaling to produce TEP cells.

33. The method of any one of claims 30-32, wherein the first cell culture medium further comprises an insulin-transferrin-selenium (ITS) supplement.

34. The method of any one of claims 30-33, wherein the first cell culture medium further comprises B27.

35. The method of any one of claims 30-34, wherein the first cell culture medium comprises a minimum essential medium or Dulbecco's minimum essential medium (DMEM).

36. The method of any one of claims 30-35, further comprising culturing the TEP cells in a second cell culture medium comprising KGF and T3 under conditions sufficient to cause further differentiation of the TEP cells to generate a population of mature TEP cells.

37. A composition comprising a differentiated population of TEP cells produced according to the method of any one of claims 1-36.

38. A composition comprising reaggregated thymic epithelial progenitor (TEP) cells differentiated from PS cells, wherein the composition further comprises one or more cell type selected from lymphatic endothelium cells, vascular endothelium cells, immune cells, mesenchymal cells, pericytes, red blood cells, or combinations thereof.

39. A composition comprising TECs differentiated from PS cells, wherein the composition further comprises one or more of cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

40. A composition comprising reaggregated thymic epithelial cells (TECs) differentiated from PS cells, wherein the composition further comprises one or more cell type selected from lymphatic endothelium cells, vascular endothelium cells, immune cells, mesenchymal cells, pericytes, red blood cells, or combinations thereof.

41. The composition of claim 40, wherein the reaggregated TECs comprise subpopulations including one or more of cTEC lineage cells, bipotent TEP cells, committed mTEC progenitor cells, immature mTECs, mature mTECs, post-AIRE mTECs, tuft cells, neuroendocrine cells, ionocytes, ciliated cells, myelin expressing cells, and/or myoid cells.

42. The method of any one of claim 1-29, 30-31, or 32-36, further comprising directing development of one or more subpopulation of thymic epithelial cells by introducing to the medium one or more of:
a. factors associated with WNT signaling;
b. factors associated with BMP signaling;
c. factors associated with TGF beta signaling;
d. factors associated with IGF signaling;
e. factors associated with FGF signaling;
f. factors associated with NOTCH signaling;
g. TNF receptors;
h. factors associated with p53 signaling; and/or
i. Toll-like receptors.

43. The method of claim 42, wherein the introducing to the medium comprises introducing a soluble form of the one or more factors and/or introducing a cell that expresses the one or more factors.

44. The method of any one of claims 16-18, wherein the thymic microenvironment is characterized by presence of cells expressing one or more of:
a. factors associated with WNT signaling;
b. factors associated with BMP signaling;
c. factors associated with TGF beta signaling;
d. factors associated with IGF signaling;
e. factors associated with FGF signaling;
f. factors associated with NOTCH signaling;
g. TNF receptors;
h. factors associated with p53 signaling; and/or
i. Toll-like receptors.

45. The method of any one of claims 42-44, wherein:
a. factors associated with WNT signaling comprise WNT5A, WNT6, ROR1, ROR2, RYK, FRZB, RSPO1, RSPO3, SFRP2, and/or SFRP5;
b. factors associated with the BMP signaling comprise BMP4, BMP5, and/or FST;
c. factors associated with TGF beta signaling comprise TGFB1, TGFBR2, CXCL12, and/or CCL21;
d. factors associated with IGF signaling comprise IGF1R;
e. factors associated with FGF signaling comprise FGFR2 and/or FGF7/KGF;
f. factors associated with NOTCH signaling comprise NOTCH1, NOTCH2, NOTCH3, HES1, HES6, DLL4, JAG2, JAG1, HES2, HES4, HEY1, NRARP, DLK1, and/or DLK2;
g. TNF receptors comprise RANK/TNFRSF11A, CD40, LTBR, TNFRSF4, TNFRSF9, LTB, and/or CD70;
h. factors associated with p53 signaling comprise PERP, SFN, CTSD, CDKN2A, and/or CDKN2B; and
i. Toll-like receptors comprise TLR1, TLR2, TLR3, TLR4, TLR5, TLR6, and/or TLR10.
